(19) United States
(12) Patent Application Publication
(10) Pub. No.: US 2025/0011722 A1
(43) Pub. Date: Jan. 9, 2025

(54) HEPATIC ORGANOIDS

(71) Applicants: Koninklijke Nederlandse Akademie van Wetenschappen, Utrecht (NL); Prinses Maxima Centrum Voor Kinderoncologie B.V., Utrecht (NL)

(72) Inventors: Johannes Carolus Clevers, Utrecht (NL); Benedetta Artegiani, Utrecht (NL); Delilah Florentine Gertrude Hendriks, Utrecht (NL)

(21) Appl. No.: 18/708,539

(22) PCT Filed: Nov. 11, 2022
(86) PCT No.: PCT/NL2022/050641

§ 371 Date: May 8, 2024

(30) Foreign Application Priority Data
Nov. 11, 2021 (GB) 2116278.9
Jun. 1, 2022 (NL) 2032049

(51) Int. Cl. C12N 5/071; A61K 31/7088; C12N 9/22; C12N 15/11

(52) U.S. Cl. CPC C12N 5/0671; A61K 31/7088; C12N 9/22; C12N 15/11; C12N 2310/20; C12N 2503/00; C12N 2513/00

(57) ABSTRACT

The invention relates to human hepatocyte organoids. In particular, hepatocyte organoids with modified genes involved in lipid homeostasis. The invention also relates to the use of such organoids for modelling diseases involving steatosis. Also included is the use of such organoids as models of fatty liver, such as caused by genetic as well as diet related fatty liver disorders and their use in discovery of novel drugs for treating fatty liver and related disorders. The invention further relates to agents for reducing steatosis in subjects having similar modifications as the hepatocyte organoids of the invention. The invention also relates to methods of reducing or preventing steatosis, treating or preventing cardiovascular disease; treating or preventing NAFLD, and preventing and/or reducing the risk of NASH and/or cirrhosis by increasing FADS2 activity in a subject. Also included is a FADS2 agonist, such as a small molecule, nucleic acid or polypeptide for use in said methods.

### FIELD OF INVENTION

The invention relates to human hepatocyte organoids. In particular, hepatocyte organoids with modified genes involved in lipid homeostasis. The invention also relates to the use of such organoids for modelling diseases involving steatosis. Also included is the use of such organoids as models of fatty liver, such as caused by genetic as well as diet related fatty liver disorders and their use in discovery of novel drugs for treating fatty liver and related disorders. The invention further relates to agents for reducing steatosis in subjects having similar modifications as the hepatocyte organoids of the invention. The invention also relates to methods of reducing or preventing steatosis, treating or preventing cardiovascular disease; treating or preventing NAFLD, and preventing and/or reducing the risk of NASH and/or cirrhosis by increasing FADS2 activity in a subject. Also included is a FADS2 agonist, such as a small molecule, nucleic acid or polypeptide for use in said methods.

### BACKGROUND

Non-alcoholic fatty liver disease (NAFLD) is a growing worldwide public health concern with over 25% of the population affected (Younossi et al., 2016). The disease has a progressive nature, starting from simple steatosis to the inflammatory subtype non-alcoholic steatohepatitis (NASH) which is characterized by fibrosis that can further worsen to cirrhosis and liver cancer (Loomba et al., 2021). NAFLD development is primarily a lifestyle disease. Dietary habits such as high caloric intake and high carbohydrate/saturated fat consumption are key risk factors (Stefan et al., 2019). The disease is epidemiologically associated with obesity, type 2 diabetes, and metabolic syndrome features (Younossi et al., 2019). Inter-individual susceptibility in NAFLD development and disease progression can be partly explained by inherited factors. Genome-wide association studies have revealed multiple NAFLD risk loci (Chambers et al., 2011; Kozlitina et al., 2014; Mancina et al., 2016; Romeo et al., 2008; Speliotes et al., 2011), with a single-nucleotide polymorphism (SNP) in the PNPLA3 gene as one of the top hits (Trépo and Valenti, 2020). NAFLD can also result from rare monogenic disorders of lipid metabolism, such as familial hypobetalipoproteinemia and abetalipoproteinemia (Liebe et al., 2021). While currently no approved drug therapies exist that can limit or reverse disease progression, multiple novel therapies acting on various metabolic targets are under evaluation (Esler and Bence, 2019). Yet, recent failures (e.g. obeticholic acid (Mullard, 2020)) underscore the complexity in combatting NAFLD.

Most of the mechanistic understanding has been obtained from mouse models through the use of genetic, chemically-induced, or diet-driven models (van Herck et al., 2017), but inherent inter-species differences complicate translating findings to humans and do not represent scalable systems. In vitro human NAFLD models instead remain scarce. The metabolism of liver cell lines does not reflect healthy liver (Zeilinger et al., 2016), while a few primary or iPSC-based NAFLD models have been recently described (Collin de l'Hortet et al., 2019; Kozyra et al., 2018; Ouchi et al., 2019; Ramli et al., 2020). However, addressing the contribution of genetics and comparing different etiologies remain largely unexplored.

Tissue-derived human 3D organoid cultures have enabled the study of tissue physiology and a diversity of infectious, hereditary, and malignant diseases (Artegiani and Clevers, 2018; Schutgens and Clevers, 2020). Combining organoids with CRISPR-Cas genome engineering further allows modelling diseases, such as cancer, and precisely addressing gene function (Hendriks et al., 2020).

Long-term expanding human hepatocyte organoid cultures have been described (Hu et al., 2018). However, these organoids have yet to provide models of lipid homeostasis related diseases leading to NAFLD.

NASH has been associated with higher mRNA expression of FADS2 for example in Xu, Yingyu, et al. “Association of non-alcoholic fatty liver disease and coronary artery disease with FADS2 rs3834458 gene polymorphism in the chinese han population. “Gastroenterology Research and Practice 2019 (2019) and Shewale, Swapnil V., et al. “Botanical oils enriched in n-6 and n-3 FADS2 products are equally effective in preventing atherosclerosis and fatty liver.” Journal of lipid research 56.6 (2015): 1191-1205. suggests that ingestion of FADS2 products may have health benefits. However, no link between FADS2 and steatosis or NAFLD has been found and no agents or methods of targeting FADS2 to treat or help prevent steatosis and the associated conditions are available.

Therefore, there is a need for a model system for studying lipid homeostasis and the diseases related thereto which can be used for identification of important genetic phenotypes and drug discovery.

### SUMMARY OF INVENTION

The investors have found that:
Genetic vs. diet-driven lipid accumulation activate differential hepatocyte responses;
Drug-mediated interference with de novo lipogenesis effectively reduces steatosis; and
FADS2 as a limiting factor for the degree of steatosis.

Non-alcoholic fatty liver disease (NAFLD) remains without cure, partly because of limitations of current experimental models. Here, the inventors model NAFLD's first stage, steatosis, in human hepatocyte organoids. It has been shown that APOB- or MTTP-mutations in organoids of the invention spontaneously yield steatosis through de novo lipogenesis. Transcriptomic comparisons between organoids of the invention and fat loaded wild type organoid models highlights major divergent hepatocyte responses. In a rational CRISPR screen, FADS2 is identified as an important lipid regulator both during homeostasis and steatosis. Collectively, these observations validate human hepatocyte organoids of the invention for studying steatosis (either genetically driven or diet driven), NAFDL, NASH and other fatty liver disorders.

The inventors have also found that combining dietary challenges with diverse CRISPR-engineering as described herein organoids that reflect 3 main disease triggers have been made: diet (free fatty acid loading), inter-individual genetic variability (PNPLA3 I148M mutation) and monogenic lipid disorders (APOB and MTTP mutations). While wild type organoids do not display steatosis under baseline conditions, isogenic organoids prime-edited to harbour PNPLA3 I148M spontaneously develop mild steatosis and possess an exacerbated response towards a dietary challenge. Organoids mutant for APOB or MTTP spontaneously develop massive steatosis, driven by accumulation of de novo-generated lipids. Furthermore, using a CRISPR loss-of-function screening platform as described herein that enabled target evaluation within 20 days. From a screen targeting 35 genes implied in lipid metabolism, fatty-acid desaturase 2 (FADS2) as found to be an important novel NAFLD determinant. While its loss aggravated steatosis, increasing FADS2 activity prevented and resolved steatosis. Mechanistically, FADS2 increases polyunsaturated fatty acid abundancy, which in turn reduces de novo lipogenesis, highlighting its potential therapeutic use. These novel human steatosis organoid models described herein allow NAFLD target discovery and form the base to model the entire disease spectrum.

In a first aspect of the invention there is provided a human hepatocyte organoid comprising at least one of:
a modified Apolipoprotein B-100 (APOB) gene;
a modified Microsomal Triglyceride Transfer Protein (MTTP) gene;
a modified FADS2 gene; and/or
a modified PNPLA3 gene.

In some embodiments, the human hepatocyte organoid comprises a modified Apolipoprotein B-100 (APOB) gene. In some embodiments, the human hepatocyte organoid embodiments, the human hepatocyte organoid comprises a modified FADS2 gene. In some embodiments, the human hepatocyte organoid comprises a modified PNPLA3 gene. In some embodiments, the human hepatocyte organoid comprises a modified Apolipoprotein B-100 (APOB) gene and a modified FADS2 gene. In some embodiments, the human hepatocyte organoid comprises a modified Apolipoprotein B-100 (APOB) gene and a modified PNPLA3 gene. In some embodiments, the human hepatocyte organoid comprises a modified Microsomal Triglyceride Transfer Protein (MTTP) gene and a modified FADS2 gene. In some embodiments, the human hepatocyte organoid comprises a modified Microsomal Triglyceride Transfer Protein (MTTP) gene and a modified PNPLA3 gene.

In some embodiments, the human hepatocyte organoid comprises a modified Apolipoprotein B-100 (APOB) gene and a modified Microsomal Triglyceride Transfer Protein (MTTP) gene.

In some embodiments, the human hepatocyte organoid comprises a modified Apolipoprotein B-100 (APOB) gene and further comprises a modified Microsomal Triglyceride Transfer Protein (MTTP) gene. In some embodiments, the human hepatocyte organoid comprises a modified Apolipoprotein B-100 (APOB) gene and further comprises a modified FADS2 gene. In some embodiments, the human hepatocyte organoid comprises a modified Apolipoprotein B-100 (APOB) gene and further comprises a modified PNPLA3 gene. In some embodiments, the human hepatocyte organoid comprises a modified Apolipoprotein B-100 (APOB) gene and further comprises at least one of a modified Microsomal Triglyceride Transfer Protein (MTTP) gene, a modified FADS2 gene, and/or a modified PNPLA3 gene

In some embodiments, the human hepatocyte organoid comprises a modified Microsomal Triglyceride Transfer Protein (MTTP) gene and further comprises a modified Apolipoprotein B-100 (APOB) gene. In some embodiments, the human hepatocyte organoid comprises a modified Microsomal Triglyceride Transfer Protein (MTTP) gene and further comprises a modified FADS2 gene. In some embodiments, the human hepatocyte organoid comprises a modified Microsomal Triglyceride Transfer Protein (MTTP) gene and further comprises a modified PNPLA3 gene. In some embodiments, the human hepatocyte organoid comprises a modified Microsomal Triglyceride Transfer Protein (MTTP) gene and further comprises at least one of a modified Apolipoprotein B-100 (APOB) gene, a modified FADS2 gene, and/or a modified PNPLA3 gene.

In some embodiments, the human hepatocyte organoid comprises a modified FADS2 gene and further comprises a modified Apolipoprotein B-100 (APOB) gene. In some embodiments, the human hepatocyte organoid comprises a modified FADS2 gene and further comprises a modified Microsomal Triglyceride Transfer Protein (MTTP) gene. In some embodiments, the human hepatocyte organoid comprises a modified FADS2 gene and further comprises a modified PNPLA3 gene. In some embodiments, the human hepatocyte organoid comprises a modified FADS2 gene and further comprises at least one of a modified Apolipoprotein B-100 (APOB) gene, a modified PNPLA3 gene, and/or a modified Microsomal Triglyceride Transfer Protein (MTTP) gene.

In some embodiments, the human hepatocyte organoid comprises a modified PNPLA3 gene and further comprises a modified Apolipoprotein B-100 (APOB) gene. In some embodiments, the human hepatocyte organoid comprises a modified PNPLA3 gene and further comprises a modified FADS2 gene. In some embodiments, the human hepatocyte organoid comprises a modified PNPLA3 gene and further comprises a modified Microsomal Triglyceride Transfer Protein (MTTP) gene. In some embodiments, the human hepatocyte organoid comprises a modified PNPLA3 gene and further comprises at least one of a modified Apolipoprotein B-100 (APOB) gene, a modified FADS2 gene, and/or a modified Microsomal Triglyceride Transfer Protein (MTTP) gene.

In some embodiments, the modification comprises a mutation or deletion.

In some embodiments, at least one of the modified Apolipoprotein B-100 (APOB) gene, the modified Microsomal Triglyceride Transfer Protein (MTTP) gene, the modified FADS2 gene, and/or the modified PNPLA3 gene are attenuated. In some embodiments, the human hepatocyte organoid comprises an attenuated Apolipoprotein B-100 (APOB) gene. In some embodiments, the human hepatocyte organoid comprises an attenuated Microsomal Triglyceride Transfer Protein (MTTP) gene. In some embodiments, the human hepatocyte organoid comprises an attenuated FADS2 gene. In some embodiments, the human hepatocyte organoid comprises an attenuated PNPLA3 gene. In some embodiments, the human hepatocyte organoid comprises an attenuated Apolipoprotein B-100 (APOB) gene and an attenuated FADS2 gene. In some embodiments, the human hepatocyte organoid comprises an attenuated Apolipoprotein B-100 (APOB) gene and an attenuated PNPLA3 gene. In some embodiments, the human hepatocyte organoid comprises an attenuated Microsomal Triglyceride Transfer Protein (MTTP) gene and an attenuated FADS2 gene. In some embodiments, the human hepatocyte organoid comprises an attenuated Microsomal Triglyceride Transfer Protein (MTTP) gene and an attenuated PNPLA3 gene.

In some embodiments, the human hepatocyte organoid comprise lipids droplets. In some embodiments, the lipid droplets occupy a greater area of the human hepatocyte organoid in comparison to a wild type human hepatocyte organoid. For example, in compression to a human hepatocyte organoid not including any of the modifications described above.

In some embodiments, human hepatocyte organoid comprises altered lipid homeostasis. For example, increased lipogenesis or decreased lipogenesis.

In some embodiments, the human hepatocyte organoid accumulates lipids via de novo lipogenesis-driven steatosis. In particular, wherein the human hepatocyte organoid includes at least one of a modified Apolipoprotein B-100 (APOB) gene and/or a modified Microsomal Triglyceride Transfer Protein (MTTP) gene.

In some embodiments, the human hepatocyte organoid is a tissue derived human hepatocyte organoid. That is to say that the organoid may be derived from tissue rather than from stem cells.

In some embodiments, the human hepatocyte organoid further comprises exogenous lipids. For example, lipids introduced into the organoid and not produced within or by the organoid itself.

In some embodiments, the human hepatocyte organoid comprises downregulation of at least one LXR-regulated gene in comparison to a wild type human hepatocyte organoid. In particular, wherein the human hepatocyte organoid includes at least one of a modified Apolipoprotein B-100 (APOB) gene and/or a modified Microsomal Triglyceride Transfer Protein (MTTP) gene.

In some embodiments, the at least one LXR-regulated gene comprises one or more of ACACA, FASN, DGAT2, SREBF1, HMGCS1, SQLE, LSS, and/or DHCR7.

In a second aspect of the invention there is provided a method of forming a human hepatocyte organoid for modelling lipid homeostasis, the method comprising:
providing a human hepatocyte organoid;
modifying at least one of:
at least one Apolipoprotein B-100 (APOB) gene; or
at least one Microsomal Triglyceride Transfer Protein (MTTP) gene;
at least one FADS2 gene; and/or
at least one PNPLA3 gene;
recovering cells comprising the modified APOB, MTTP, FADS2, and/or PNPLA3 genes; and
culturing the cells to form human hepatocyte organoids.

In some embodiments, modifying comprises CRISPR based gene disruption.

In some embodiments, CRISPR based gene disruption comprises introducing into cells of the human hepatocyte organoid one or more vectors for disrupting the APOB, MTTP FADS2, and/or PNPLA3 genes, the at least one vector comprising at least one of a guide RNA for targeting APOB, MTTP FADS2, and/or PNPLA3 and/or a Cas9 enzyme.

In a third aspect of the invention there is provided use of a human hepatocyte organoid of the invention for modelling lipid homeostasis. In some embodiments the use further comprises drug discovery and/or CRISPR based screening of lipid homeostasis mediators. In some embodiments there is provided the use of a human hepatocyte organoid of the invention for drug discovery. In some embodiments there is provided the use of a human hepatocyte organoid of the invention for CRISPR screening.

In some embodiments, the human hepatocyte organoid is for modelling steatosis

In some embodiments, the steatosis is de novo lipogenesis driven steatosis.

In some embodiments, the human hepatocyte organoid is for modelling NAFLD, NASH and/or liver cancer. For example, for modelling NAFDL. For example, modelling NASH. For example, modelling liver cancer.

In a fourth aspect of the invention there is provided a p38 inhibitor, FADS2 agonist, ACC inhibitor, DGAT2 inhibitor, FAS inhibitor, recombinant hFGF19 or FXR agonist for use in treating NAFLD in a subject in need thereof, wherein the subject comprises at least one of:
at least one modified PNPLA3 gene;
at least one modified FADS2 gene;
at least one modified APOB gene; and/or
at least one modified MTTP gene.

In some embodiments, at least one of the modified Apolipoprotein B-100 (APOB) gene, the modified Microsomal Triglyceride Transfer Protein (MTTP) gene, the modified FADS2 gene, and/or the modified PNPLA3 gene are attenuated. In some embodiments, the subject comprises an attenuated Apolipoprotein B-100 (APOB) gene. In some embodiments, the subject comprises an attenuated Microsomal Triglyceride Transfer Protein (MTTP) gene. In some embodiments, the subject comprises an attenuated FADS2 gene. In some embodiments, the subject comprises an attenuated PNPLA3 gene. In some embodiments, the subject comprises an attenuated Apolipoprotein B-100 (APOB) gene and an attenuated FADS2 gene. In some embodiments, the subject comprises an attenuated Apolipoprotein B-100 (APOB) gene and an attenuated PNPLA3 gene. In some embodiments, the subject comprises an attenuated Microsomal Triglyceride Transfer Protein (MTTP) gene and an attenuated FADS2 gene. In some embodiments, the subject comprises an attenuated Microsomal Triglyceride Transfer Protein (MTTP) gene and an attenuated PNPLA3 gene.

In some embodiments, the modified gene is a modified gene as described herein.

In some embodiments, the subject suffers from familial hypobetalipoproteinaemia (FHBL).

In some embodiments, the familial hypobetalipoproteinaemia is associated with the least one attenuating APOB mutation.

In some embodiments, the subject suffers from abetalipoproteinemia (ABL).

In some embodiments, the abetalipoproteinemia is associated with the at least one attenuating MTTP mutation.

In some embodiments, the at least one modified PNPLA3 comprises a homozygous or heterozygous PNPLA3 I148M mutation.

In some embodiments, the at least one modified FADS2 comprises a single nucleotide polymorphism.

In a fifth aspect of the invention there is provided a method reducing steatosis in hepatocytes in a subject in need thereof, comprising administering an agent targeting de novo lipogenesis to the subject, wherein the subject comprises at least one of:
at least one modified PNPLA3 gene;
at least one modified FADS2 gene;
at least one modified APOB gene; and/or
at least one modified MTTP gene.

In some embodiments, at least one of the modified Apolipoprotein B-100 (APOB) gene, the modified Microsomal Triglyceride Transfer Protein (MTTP) gene, the modified FADS2 gene, and/or the modified PNPLA3 gene are attenuated. In some embodiments, the subject comprises an attenuated Apolipoprotein B-100 (APOB) gene. In some embodiments, the subject comprises an attenuated Microsomal Triglyceride Transfer Protein (MTTP) gene. In some embodiments, the subject comprises an attenuated FADS2 gene. In some embodiments, the subject comprises an attenuated PNPLA3 gene. In some embodiments, the subject comprises an attenuated Apolipoprotein B-100 (APOB) gene and an attenuated FADS2 gene. In some embodiments, the subject comprises an attenuated Apolipoprotein B-100 (APOB) gene and an attenuated PNPLA3 gene. In some embodiments, the subject comprises an attenuated Microsomal Triglyceride Transfer Protein (MTTP) gene and an attenuated FADS2 gene. In some embodiments, the subject comprises an attenuated Microsomal Triglyceride Transfer Protein (MTTP) gene and an attenuated PNPLA3 gene.

In some embodiments, the agent comprises at least one of:
p38 inhibitor;
FADS2 agonist;
ACC inhibitor;
FXR agonist;
FAS inhibitor;
DGAT2 inhibitor; and/or
recombinant hFGF19.

In some embodiments, the subject suffers from NAFLD.

In a sixth aspect of the invention there is provided a method of treating NAFLD comprising inducing Dual Specificity Phosphatase 4 and/or Dual Specificity Phosphatase 5 in a subject in need thereof.

In some embodiments, inducing Dual Specificity Phosphatase 4 and/or Dual Specificity Phosphatase 5 comprises administering an agent that inhibits p38 signalling.

In one aspect of the invention there is provided a FADS2 agonist for use in treating or preventing NAFLD in a subject in need thereof.

In one aspect there is provided a FADS2 agonist for use in treating or preventing a cardiovascular disease in a subject in need thereof.

In one aspect there is a provided a method of treating or preventing a cardiovascular disease in a subject in need thereof, comprising increasing FADS2 activity in the subject.

In one aspect there is provided a FADS2 agonist for use in reducing and/or preventing steatosis in a subject in need thereof.

In one aspect there is provided a FADS2 agonist for use in preventing and/or reducing the risk of NASH and/or cirrhosis in a subject in need thereof.

In one aspect there is provided a method of preventing and/or reducing the risk of NASH and/or cirrhosis in a subject in need thereof, comprising increasing FADS2 activity in the subject.

In one aspect there is a provided a method of reducing and/or preventing steatosis in a subject in need thereof, comprising increasing FADS2 activity in the subject.

In one aspect there is a provided a method of treating or preventing NAFLD in a subject in need thereof, comprising increasing FADS2 activity in the subject.

In some embodiments, increasing FADS2 activity comprises increasing expression of an endogenous FADS2 of the subject.

In some embodiments increasing FADS2 activity comprises increasing activity of an endogenous FADS2 gene and/or polypeptide in the subject.

In some embodiments, increasing FADS2 activity comprises administering a FADS2 agonist to the subject.

In some embodiments, the steatosis is dietary induced steatosis.

In some embodiments the FADS2 agonist comprises an agent for increasing endogenous FADS2 activity in the subject. In some embodiments, the agent comprises an agent for increasing exogenous FADS2 activity in the subject. In some embodiments the FADS2 agonist comprises an agent for increasing activity of an endogenous and/or exogenous FADS2 polypeptide and/or gene in the subject. For example, the agent may be a small molecule, a nucleic acid and/or a polypeptide. In some embodiments, the FADS2 agonist comprises an agent that increases activity of an endogenous FADS2 polypeptide. In some embodiments, the FADS2 agonist comprises an agent that increases activity of an exogenous FADS2 polypeptide. For example, increases activity in comparison to a subject that has not received a FADS2 agonist.

In some embodiments the agent comprises a nucleic acid that encodes a FADS2 agonist. For example, the nucleic acid may encode an exogenous and/or endogenous FADS2 polypeptide. In some embodiments, the nucleic acid may comprises a nucleotide sequence encoding a human FADS2 polypeptide. For example, a nucleic acid encoding the sequence of FADS2 as identified by the UniProtKB number 095864 or an isoform thereof.

In some embodiments, the agent comprises a polypeptide encoding an exogenous and/or endogenous FADS2 polypeptide. In some embodiments, the polypeptide comprises a human FADS2 polypeptide. For example, a polypeptide as identified by the UniProtKB number O95864 or an isoform thereof. For example a FADS2 comprising the amino acid according to any one of SEQ ID NOs: 31-34.

In some embodiments, the FDS2 agonist comprises an expression vector. In some embodiments, the expression vector comprises a nucleic acid encoding a FADS2 polypeptide or gene as described herein. For example, a human FADS2 polypeptide.

In some embodiments, the FADS2 agonist may comprise a gene editing system suitable for altering the activity of FADS2 in the subject. For example, one or more nucleic acids that encode a CRISPR system that targets a FADS2 gene of the subject and increases expression of a FADS2 polypeptide.

In some embodiments, increasing FADS2 activity and/or the FADS2 agonist increase the amount of triacylglycerides comprising a chain length of at least 54 carbons in the subject.

In some embodiments, increasing FADS2 activity and/or the FADS2 agonist increase the amount of unsaturated triacylglycerides and/or increase the level of unsaturation of triacylglycerides in the subject.

In some embodiments, increasing FADS2 activity and/or the FADS2 agonist decrease the amount of fatty acids in the subject.

In some embodiments, increasing FADS2 activity and/or the FADS2 agonist decrease the de novo lipogenesis (DNL) index of the subject.

In certain embodiments, the subject comprises at least one of:
at least one modified PNPLA3 gene;
at least one modified FADS2 gene;
at least one modified APOB gene; and/or
at least one modified MTTP gene as described herein.

In some embodiments, the subject suffers from a monogenic lipid disorder. For example, familial hypobetalipoproteinaemia (FHBL) and abetalipoproteinemia (ABL).

In some embodiments, the subject does not suffer from NASH.

In some embodiments, the FADS2 agonist is part of a pharmaceutical composition.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country. Various aspects of the invention are described in further detail below.

### BRIEF DESCRIPTION OF FIGURES

Embodiments of the invention are further described hereinafter with reference to the accompanying drawings, in which:

FIG. 1 shows generation of CRISPR-engineered human steatosis organoids. (a) Schematic representation of the roles of ApoB (APOB) and MTP (MTTP) in the generation of VLDL particles within the hepatocyte; (b) Brightfield images of wild type and APOB^{-/-} human hepatocyte organoids; (c) Nile Red lipid staining of wild type and APOB^{-/-} organoids, cell boundaries are marked by phalloidin staining; (d) Quantifications of the number of lipid droplets and the organoid area covered by the lipid droplets in wild type and APOB^{-/-} organoids. Dots represent individual organoids and the different colors indicate different lines. Analyses were performed in 3 lines from 3 donors; (e) Transmission electron microscopy of wild type and APOB^{-/-} organoids. Asterisks mark the presence of lipid droplets residing within the nucleus in APOB^{-/-} organoids; (f) Brightfield image of MTTP^{-/-} organoids; (g) Nile Red lipid staining of MTTP^{-/-} organoids, cell boundaries are marked by phalloidin staining; and (h) Quantifications of the number of lipid droplets and the organoid area covered by the lipid droplets in MTTP^{-/-} organoids. Dots represent individual organoids and the different colors indicate different lines. Analyses were performed in 3 lines from 2 donors;

FIG. 2 shows characterization of APOB^{-/-} and MTTP^{-/-} organoids. (a) Schematic representation of CRISPR editing at the APOB locus. An example of the genotype of a clonal APOB^{-/-} organoid line is shown. (b) Immunofluorescent images of ApoB expression in wild type and APOB^{-/-} organoids. (c) Nile Red lipid staining of wild type (APOB^{+/+}), ApoB heterozygous (APOB^{+/-}), and ApoB homozygous (APOB^{-/-}) organoids. (d) H&E staining of an APOB^{-/-} organoid. Note the presence of lipid droplets. (e) Immunofluorescent images of Ki-67 in wild type and APOB^{-/-} organoids. On the right, quantification of the amount of Ki-67⁺ cells within an organoid. Dots represent individual organoids. n.s.=not significant. (f) Immunofluorescent images of hepatocyte (ALB, MRP2, HNF4A) and structural (B-CAT) markers in APOB^{-/-} organoids. Note the presence of binucleated cells. (g) Schematic representation of CRISPR editing at the MTTP locus. An example of the genotype of a clonal MTTP^{-/-} organoid line is shown. (h) Immunofluorescent images of MTP expression in wild type and MTTP^{-/-} organoids;

FIG. 3 shows lipidomic and transcriptomic characterization of APOB^{-/-} and wild type organoids (a) Schematic representation of the workflow for lipidomic analyses; (b) Principle component analysis of neutral lipids in the supernatant and intracellularly in APOB^{-/-} and wild type organoids using APCI; (c) Quantification of the total neutral lipid signal (peak intensity) detected in the supernatant and intracellularly in APOB^{-/-} and wild type organoids using APCI. Relative compositions of the different neutral lipid classes are given in pie charts; (d) Quantification of the absolute abundancy of the different neutral lipid classes in APOB^{-/-} organoids relative to wild type organoids; (f) Heatmap of DEGs in APOB^{-/-} vs. wild type organoids in 3 lines from 3 different donors (log 2FC>0.5, p<0.005); (g) Volcano plot of DEGs in APOB^{-/-} organoids (log 2FC>0.5, p<0.005); (h) Expression levels of selected genes involved in de novo lipogenesis (FASN, ACACA, SREBF1) and cholesterol biosynthesis (HMGCS1, LSS, DHCR7). Normalized transcript levels from bulk RNA sequencing are plotted. Different shaped dots represent the different donors;

FIG. 4 shows lipidomic and transcriptomic profiling of APOB^{-/-} organoids (a-b) Surface plots of intracellular and secreted lipid species detected in wild type organoids and APOB^{-/-} organoids using (a) APCI and (b) HESI. (c) Distribution of TAG saturation across secreted and intracellular lipid species in wild type and APOB^{-/-} organoids using scaled HESI data. (d) Distribution of TAG chain length across secreted and intracellular lipid species in wild type and APOB^{-/-} organoids using scaled HESI data. (e) Hierarchical clustering and heatmap depicting z-scores of most differentially abundant TAG species in APOB^{-/-} organoids vs. wild type organoids using scaled HESI data. (f) Selected GO terms of DEGs in APOB^{-/-} organoids (log 2FC>0.5, p<0.005). m.p.=metabolic process, b.p.=biological process. (g) Selected DisGeNET associations of DEGs in APOB^{-/-} organoids (log 2FC>0.5, p<0.005). (h) Expression levels of selected LXR target genes. Normalized transcript levels from bulk RNA sequencing are plotted (log 2FC>0.5, p<0.005). Different shaped dots represent the different donors. (i) Expression levels of CYP27A1 and CYP3A7 in APOB^{-/-} organoids relative to the matched wild type controls derived from normalized transcript levels from bulk RNA sequencing data (log 2FC>0.5, p<0.005). Different shaped dots represent the different donor.

FIG. 5 shows transcriptomic comparisons between APOB^{-/-} organoids and fat-loaded wild type organoids (a) Schematic representation of the experimental procedure to generate FFA-loaded wild type organoids; (b) Nile Red lipid staining of FFA-loaded wild type organoids; (c) and (d) Quantifications of the number of lipid droplets and the organoid area covered by the lipid droplets in FFA-loaded wild type organoids. Dots represent individual organoids and the different colors indicate different lines. Analyses were performed in 3 lines from 3 donors; (e) Heatmap of DEGs in FFA-loaded vs. wild type organoids in 2 lines from 2 different donors (log 2FC>0.5, p<0.005); (f) Volcano plot of DEGs in FFA-loaded wild type organoids (log 2FC>0.5, p<0.005); (g) Expression levels of selected genes involved in de novo lipogenesis (FASN), glycerolipid synthesis (GPAM), fatty acid oxidation (CPT1A), lipid metabolism (APOA4), ketogenesis (HMGCS2) and the metabolic transcription factor PPARA. Normalized transcript levels from bulk RNA sequencing are plotted. Different shaped dots represent the different donors; (h) Venn-diagram displaying the overlap and condition-specific DEGs between APOB^{-/-} organoids and FFA-loaded wild type organoids (log 2FC>0.5, p<0.005); (i) Lists of selected DEGs related to lipid metabolism unique to APOB^{-/-} organoids or FFA-loaded wild type organoids as well as DEGs in common with opposite or similar directionality (log 2FC>0.5, p<0.005);

FIG. 6 shows transcriptomic profiling of fat-loaded wild type organoids and comparisons with APOB^{-/-} organoids. (a) Selected GO terms of DEGs in FFA-loaded wild type organoids (log 2FC>0.5, p<0.005). m.p.=metabolic process, b.p.=biological process, c.p.=catabolic process. (b) Selected DisGeNET associations of DEGs in FFA-loaded wild type organoids (log 2FC>0.5, p<0.005). (c) Hierarchical clustering and heatmap depicting z-scores of genes involved in PPAR signaling in FFA-loaded vs. wild type organoids in 2 lines from 2 different donors. (d) Volcano plot illustrating DEGs involved in fibrogenesis and activation of hepatic non-parenchymal cells in FFA-loaded wild type organoids (log 2FC>0.5, p<0.005). (e) Hierarchical clustering and heatmap depicting z-scores of genes involved in cell cycle and DNA replication in FFA-loaded vs. wild type organoids in 2 lines from 2 different donors. (f) Expression levels of selected Wnt target genes. Normalized transcript levels from bulk RNA sequencing are plotted (log 2FC>0.5, p<0.005). Different shaped dots represent the different donors. (g) Volcano plot illustrating the log 2FC of genes within APOB^{-/-} organoids vs. wild type organoids (x-axis) and FFA-loaded wild type organoids vs. wild type organoids (y-axis). (h) Hierarchical clustering and heatmap depicting z-scores of genes involved in the metabolism of lipids and lipoproteins in FFA-loaded, APOB^{-/-}, and wild type organoids from the same donor. (i) Hierarchical clustering and heatmap depicting z-scores of genes involved in cholesterol homeostasis in FFA-loaded, APOB^{-/-}, and wild type organoids from the same donor;

FIG. 7 shows anti-NAFLD drug screening in genetic and diet-induced human steatosis organoid models (a) Schematic representation of anti-NAFLD metabolic targets and their function within a hepatocyte; (b) Schematic representation of the drug screening assay in the different steatosis organoid models; (c) Examples of Nile Red lipid stainings of APOB^{-/-} organoids treated with different drugs for 7 days; (d) Lipid score analysis of the screened drugs in the different steatosis organoid models. The lipid score is determined by the lipid droplet fluorescence and area coverage on a linear scale from 0 (wild type organoids) to 1 (vehicle-treated APOB^{-/-} or MTTP^{-/-} organoids or FFA-loaded vehicle-treated wild type organoids); (e) Brightfield and fluorescent images of a PLIN2::mNEON; APOB^{-/-} reporter organoid. Note the overlap between the fluorescent PLIN2 signal and the brightfield lipid droplets; (f) Brightfield and fluorescent images of a PLIN2::tdTomato; MTTP^{-/-} organoid culture treated with ACCi or vehicle for 5 days; (g) Quantification of drug effects using the fluorescence signal from the PLIN2::tdTomato; MTTP^{-/-} reporter as a read-out. The mean response of 2 independent measurements within the same line at the different indicated days is shown;

FIG. 8 shows drug screening in steatosis organoids. (a) Nile Red staining of MTTP^{-/-} organoids exposed to different drugs at increasing drug concentrations. (b) Time-lapse brightfield images of 3 different APOB^{-/-} organoids exposed to ACCi over a 78 h window. (c) Effect of combined DGAT1i and DGAT2i exposure in APOB^{-/-}, MTTP^{-/-}, and FFA-loaded wild type organoids. Brightfield images and Nile Red staining are from treated APOB^{-/-} organoids. (d) Schematic representation of the workflow to generate endogenous PLIN2-tagged reporter organoids. (e) Examples of genotypes of PLIN2::mNEON and PLIN2::tdTomato reporter lines, confirming precise integration of the fluorescent tags at the C-terminus of PLIN2. (f) Co-staining of PLIN2::tdTomato::MTTP^{-/-} organoids with BioTracker 488 Green Lipid Droplet Dye demonstrating the overlap between PLIN2 fluorescent signal and lipid droplets. (g) Lipid droplet area coverages in PLIN2::tdTomato; MTTP^{-/-} and PLIN2::mNEON; MTTP^{-/-} relative to untagged MTTP^{-/-} organoids from the same background line. Dots represent individual organoids. n.s.=not significant. (h) Time-lapse fluorescent images of PLIN2::tdTomato; MTTP^{-/-} organoids treated with different drugs over a 7 day window;

FIG. 9 shows transcriptomic evaluation of the mechanism of action and cellular responses of anti-NAFLD drug (a) Heatmap of DEGs upon the different drug treatments. All genes that were at least significantly differentially expressed in one treatment are plotted (log 2FC>0.5, p<0.005); (b-f) Volcano plot of DEGs upon (b) ACCi, (c) FASi, (d) FXRa, (e) hFGF19, and (f) DGAT2i (log 2FC>0.5, p<0.005); (g) Venn diagram displaying the overlap and unique DEGs between drug treatments (log 2FC>0.5, p<0.005); (h) List of selected DEGs in common between ACCI, FASi, FXRa, and hFGF19 treatment (log 2FC>0.5, p<0.005); (i) Expression levels of DUSP4 and DUSP5 upon different drug treatments. Normalized transcript levels from bulk RNA sequencing are plotted; (j) Schematic representation of DUSP proteins acting on different MAPK signaling pathways; (k) Nile Red lipid staining of APOB^{-/-} organoids treated with different MAPK inhibitors; (l) Lipid score analysis of MAPK inhibitor effects in the different steatosis organoid models;

FIG. 10 shows transcriptomic changes induced by different anti-NAFLD drugs. (a) Expression levels of genes involved in de novo lipogenesis in wild type organoids and vehicle-, ACCi-, or FASi-treated APOB^{-/-} organoids from the same donor. (b) List of notable DEGs comparing ACCi vs. FASi treatment in APOB^{-/-} organoids (log 2FC>0.5, p<0.005). (c) Volcano plots illustrating differential gene expression of typical FXR target genes in FXRa- and hFGF19-treated APOB^{-/-} organoids. (d) List of notable DEGs comparing FXRa vs. hFGF19 treatment in APOB^{-/-} organoids (log 2FC>0.5, p<0.005). (e) Hierarchical clustering and heatmap depicting z-scores of genes involved in TGFβ regulation of extracellular matrix upon FXRa and hFGF19 treatment in APOB^{-/-} organoids. (f) Expression levels of hepatocyte markers ALB and TTR. Normalized transcript levels from bulk RNA sequencing are plotted (log 2FC>0.5, p<0.005). Different shaped dots represent the different donors.

FIG. 11 CRISPR screening in APOB^{-/-} and MTTP^{-/-} organoids (a) Schematic representation of the experimental procedure to perform CRISPR screening in APOB^{-/-} and MTTP^{-/-} organoids. Per condition, one individual gene is targeted; (b) Brightfield images of outgrowing APOB^{-/-} organoids from transfection with Cas9-DGAT2 sgRNA 10- and 25-days post electroporation. Asterisks indicate organoids with a visibly different (fat-free) phenotype than other outgrowing organoids; (c) Brightfield images and Nile Red lipid staining of CRISPR-targeted APOB^{-/-} and MTTP^{-/-} organoids. Boxes in the FASN and ACACA/ACACB sgRNA condition point out the tiny fat-free outgrowing organoids; (d) Brightfield image of outgrowing APOB^{-/-} organoids from transfection with Cas9-FADS2 sgRNA 25-days post electroporation. Asterisks indicate organoids with a visibly different (fatter) phenotype than other outgrowing organoids; (e) Brightfield images and Nile Red lipid staining of a MTTP^{-/-}; FADS2^{-/-} line; (f) Quantification of the organoid area covered by the lipid droplets in MTTP^{-/-}; FADS2^{-/-} organoids. Dots represent individual organoids and the different colors indicate different lines generated from the same background MTTP^{-/-} line; (g) Brightfield images and Nile Red lipid staining of a FADS2^{-/-} line generated in wild type organoids; (h) Quantification of the organoid area covered by the lipid droplets in FADS2^{-/-} organoids. Dots represent individual organoids and the different colors indicate different lines generated from the same donor;

FIG. 12 Characterization of CRISPR-screened genes in APOB^{-/-} and MTTP^{-/-} organoids. (a) Schematic representation of CRISPR editing at the DGAT2 locus. Examples of the genotype of a clonal APOB^{-/-}; DGAT2^{-/-} organoid line and a clonal APOB^{-/-}; DGAT2^{+/+} with corresponding brightfield images and Nile Red staining of the cultures are shown. (b) Immunofluorescent staining of Ki-67 in APOB^{-/-}; DGAT2^{-/-} organoids, cell membranes are marked by B-CAT. On the right, quantification of the amount of Ki-67+ cells within an organoid. Dots represent individual organoids. (c) Quantification of the number of lipid droplets and organoid area covered by the lipid droplets in wild type, APOB^{-/-}, and APOB^{-/-}; DGAT2^{-/-} organoids from the same donor. Dots represent individual organoids. ***p<0.001. (d) Expression levels of cell cycle and DNA replication genes upon ACCi and FASi treatment in APOB^{-/-} organoids. Normalized transcript levels from bulk RNA sequencing are plotted (log 2FC>0.5, p<0.005). Different shaped dots represent the different donors. (e) Schematic representation of CRISPR editing at the FADS2 locus. An example of the genotype of a clonal MTTP^{-/-}; FADS2^{-/-} organoid line is shown. (f) Brightfield image and Nile Red lipid staining of an APOB^{-/-}; FADS2^{-/-} line;

FIG. 13 Generation and drug responses of prime-edited PNPLA3 variant organoids. (a) Volcano plot illustrating differential gene expression of putative NAFLD risk genes in APOB^{-/-} organoids (log 2FC>0.5, p<0.005). (b) Schematic representation of CRISPR editing at the PNPLA3 locus. An example of the genotype of a clonal MTTP^{-/-}; PNPLA3^{-/-} organoid line is shown. (c) Nile Red lipid staining of an APOB^{-/-}; PNPLA3^{-/-} line. (d) Schematic representation of PE3-based prime editing to introduce the PNPLA3 I148M and I148* mutations. (e) Examples of the genotypes of prime-edited clonal heterozygous and homozygous I148M lines as well as homozygous I148* lines generated in a background PNPLA3^{I148I/I148I} line. (f) Prime editing efficiencies editing at the PNPLA3 locus to introduce the I148M and I148* mutations using PE3. (g) Brightfield images of the different prime-edited PNPLA3 variant organoids. Arrows point toward visible lipid droplets. (h) Nile Red lipid staining of different prime-edited PNPLA3 variants exposed to DGAT2i, FASi, or vehicle for 7 days;

FIG. 14 Interrogation of PNPLA3 function and the consequences of the I148M variant (a) Nile Red lipid staining of a MTTP^{-/-} line and the same line upon knock-out of PNPLA3 (MTTP^{-/-}; PNPLA3^{-/-}); (b) Quantification of the organoid area covered by the lipid droplets in MTTP^{-/-}; PNPLA3^{-/-} organoids. Dots represent individual organoids and the different colors indicate different lines generated from the same background MTTP^{-/-} line; (c) Brightfield images and Nile Red lipid staining of a PNPLA3^{-/-} line generated in wild type organoids; (d) Quantification of the organoid area covered by the lipid droplets in PNPLA3^{-/-} organoids. Dots represent individual organoids and the different colors indicate different lines generated from the same donor; (e) Nile Red lipid staining of different prime-edited PNPLA3 variant organoid lines derived from the same donor, cell boundaries are marked by phalloidin staining; (f) Quantification of the organoid area covered by the lipid droplets in the different PNPLA3 variant organoids. Dots represent individual organoids and the different colors indicate different lines generated from the same donor; (g) Brightfield images and Nile Red lipid staining of different PNPLA3 variants challenged with exogenous fat for 5 days;

FIG. 15 shows A) Representative brightfield images of FatTracer (with intact FADS2) and a clonal FatTracer; FADS2^{-/-} line. Note the increased abundancy of lipid droplets upon loss of FADS2. B) Representative Nile Red lipid staining of FADS2^{WT} and FADS2^{-/-} organoids in an otherwise wild type background under homeostasis (basal culture conditions) and after FFA exposure (320 μM). C) Quantification of the percentage of spontaneous steatosis (left) and upon FFA exposure (320 μM) (right) in FADS2^{WT} and FADS2^{-/-} organoids in a wild type background. Each dot represents quantification in an organoid, with n≥3 organoids from 2 different clonal lines for both conditions. ***p<0.001. D) Representative brightfield images and Nile Red lipid staining of FatTracer organoids under FADS2−/− (KO), FADS2WT, and FADS2OE conditions, demonstrating that the presence of FADS2 dictates steatosis levels within the hepatocytes. E) Representative Nile Red lipid staining of FADS2WT and FADS2OE organoids in an otherwise wild type background after FFA exposure (500 μM). F) Quantification of the percentage of steatosis upon FFA exposure (500 μM) in FADS2OE and FADS2WT organoids in a wild type background. Each dot represents quantification in an organoid, with n=4 organoids from 2 different clonal lines per condition. ***p<0.001. G) Quantification of the percentage of steatosis in FatTracer organoids under FADS2−/−, FADS2WT, and FADS2OE conditions. Each dot represents quantification in an organoid, with n=3 organoids from 2-3 different clonal lines per condition. ***p<0.001. H) Quantification of the TAG content in FatTracer organoids under FADS2−/− and FADS2OE conditions relative to FADS2WT. Data are derived from quantification in 3 different clonal lines per condition with n=2 technical replicates. **p<0.01, ***p<0.001. I) Bar plots showing the relative distribution of degree of TAG unsaturation (0 indicating all 3 fatty acids within a TAG to be saturated) in FatTracer organoids under FADS2−/−, FADS2WT and FADS2OE. Note the enrichment of highly unsaturated TAG in FADS2OE organoids, while conversely lowly unsaturated TAGs are enriched in FADS2KO organoids. Data are derived from quantification in 3 different clonal lines per condition with n=2 technical replicates. J) Bar plots showing the relative distribution of degree of TAG chain length in FatTracer organoids under FADS2−/−, FADS2WT and FADS2OE 814. Note the enrichment of long-chain TAGs in FADS2OE organoids. Data are derived from quantification in 3 different clonal lines per condition with n=2 technical replicates. K) Quantification of the de novo lipogenesis (DNL) index calculated based on the C16:0/C18:2 ratio in FatTracer organoids under FADS2−/−, FADS2WT and FADS2OE. C16:0, palmitic acid, represents the main lipogenesis product and C18:2, linolenic acid, represents the diet-derived. L) Schematic representing the proposed mechanism of FADS2 action in regulating steatosis by modulating PUFA abundancy within the hepatocytes, resulting in repression of de novo lipogenesis and ultimately reducing the TAG and thus fat content.

FIG. 16 shows FADS2 overexpression alleviates steatosis in FatTracer. A) Schematic of the constructs used to overexpress FADS2 or FADS2-P2A-tdTomato using a transposon-based strategy. B) Representative brightfield images of outgrowing FatTracer organoids upon transfection with the FADS2 overexpression construct. Asterisks highlight the appearance of lighter-less lipid containing organoids. Data are representative of multiple transfections performed with both APOB^{-/-} and MTTP^{-/-} organoids as FatTracer from 2 different donors. C) Representative brightfield and RFP images of outgrowing FatTracer organoids upon transfection with the FADS2-P2A-tdTomato overexpression construct. The asterisk highlights the appearance of a lighter—less lipid containing—organoid that is, as expected, also RFP⁺. D) FACS analysis of single cells from a FatTracer culture transfected with the FADS2-P2A-tdTomato construct 25 days p.e., as well as single cells from wild type organoids using the BioTracker 488 Green Lipid Dye, demonstrating that FADS2-overexpressing FatTracer cells (tdTomato⁺) show reduced lipid droplet intensity (nearing wild type cells) as compared to FatTracer cells not overexpressing FADS2 (tdTomato⁻). E) Schematic representation of the PCR amplification area to confirm genomic integration of FADS2 (top) and representative gel results demonstrating integration of the construct with the expected size in different FatTracer; FADS2^{OE} lines (bottom). F) Level of FADS2 overexpression in different FatTracer; FADS2^{OE} lines relative to FatTracer as determined by qPCR analysis. G) Representative brightfield images of FatTracer and FatTracer; FADS2^{OE} lines, demonstrating the near fat-free phenotypes upon FADS2 overexpression. H) Representative immunofluorescence staining for Ki-67 and β-catenin in FatTracer; FADS2^{OE} organoids, demonstrating intact proliferation upon FADS2 overexpression. I) Representative Nile Red lipid staining of FatTracer lines with various degrees of FADS2 overexpression, demonstrating that a threshold of amount of FADS2 is needed for steatosis-reducing effects

FIG. 17 shows Lipidomic analyses of FADS2 variant FatTracer organoids. A) Heatmap displaying the absolute abundancy (log 2-transformed) of all detected TAG species in FADS2^{-/-} (KO), FADS2^{WT}, and FADS2^{OE} FatTracer organoids. Data are derived from the average abundancy of 3 different clonal lines per condition with n=2 technical replicates. B) Heatmap displaying the TAG species abundancy (Z-score values) in FADS2^{-/-}, FADS2^{WT}, and FADS2^{OE} FatTracer organoids. Data are derived from the average abundancy of 3 different clonal lines per condition with n=2 technical replicates. C) Bar plots depicting the relative TAG species abundancy within FADS2^{-/-}, FADS2^{WT}, and FADS2^{OE} FatTracer organoids. The mean+SD of 3 different clonal lines per condition with n=2 technical replicates is shown. D) Heatmap displaying the absolute abundancy (log 2-transformed) of all detected fatty acid (FA) species in FADS2^{-/-}, FADS2^{WT}, and FADS2^{OE} FatTracer organoids. Data are derived from the average abundancy of 3 different clonal lines per condition with n=2 technical replicates. E) Heatmap displaying the FA abundancy (Z-score values) in FADS2^{-/-}, FADS2^{WT}, and FADS2^{OE} FatTracer organoids. Data are derived from the average abundancy of 3 different clonal lines per condition with n=2 technical replicates. F) Bar plots depicting the relative FA abundancy within FADS2^{-/-}, FADS2^{WT}, and FADS2^{OE} FatTracer organoids. The mean+SD of 3 different clonal lines per condition with n=2 technical replicates is shown.

FIG. 18 shows Organoid models of de novo lipogenesis-generated lipid accumulation by introducing APOB or MTTP mutations. a) Workflow to perform lipidomics on organoid cultures. b) Principle component analysis on the neutral lipids found in the supernatant of wild type organoids and in blank medium. Each dot represents an independent measurement. n=4 wild type organoid cultures from 2 different donors; n=2 for blank medium. c) Quantification of the TAG content present in the supernatant of wild type organoids relative to the TAG amount in blank medium. n=4 wild type organoid cultures from 2 different donors and n=2 for blank medium. ***p<0.001; t-test. d) Principle component analysis on the neutral lipids found intracellularly and in the supernatant of wild type and APOB^{-/-} organoids. Each dot represents an independent measurement. n=4 APOB^{-/-} organoid cultures from 2 different donors; n=8 wild type organoid cultures from the same 2 donors. e) Workflow to perform de novo lipogenesis tracing in APOB^{-/-} organoids. f) Quantification of the percentage of glucose-driven DNL contribution to the fatty acid pool at different time points upon [U-¹³C]-glucose tracing in APOB^{-/-} organoids. The mean+SD from independent quantifications in n=2 organoid cultures from 2 different donors per timepoint is shown. f) Quantification of the percentage of glucose-driven DNL contribution for the five non-essential (n.e.) fatty acids that can be formed by DNL in APOB^{-/-} organoids 5 days post tracing. As expected, no labelling is observed for C20:4, which is an essential (e.) fatty acid (i.e. those that cannot be synthesized by the cells). Sample sizes as in f.

FIG. 19 shows Lipidomic characterization of wild type and APOB^{-/-} organoids. a) Surface plots of neutral lipid species detected intracellularly and in the supernatant of wild type organoids and comparison with blank medium, demonstrating active VLDL secretion in wild type organoids. b) Heatmap displaying the absolute abundancy (log 2-transformed) of TAG species in the supernatant of 3-day-cultured wild type organoids and comparison with blank medium. Each column is an independent replicate. n=4 organoid cultures from 2 different donors; n=2 for blank medium. c) Representative brightfield images and Nile Red lipid staining of medium withdrawal experiments confirm that withdrawal of putative lipid sources in the medium (e.g. RSPO1-conditioned medium (CM)) do not alter the steatosis phenotype of APOB^{-/-} organoids. Scale bar=200 μm (brightfield images) and 50 μm (fluorescence images). d) Representative filipin III staining marking free cholesterol in wild type and APOB^{-/-} organoids, demonstrating the predominant presence on the membrane. Scale bar=50 μm. e) Representative mass spectra highlighting the selectivity in [U-¹³C]-glucose incorporation between the non-essential (C16:1) and essential (C20:4) fatty acid at day 1 (top) and day 5 (bottom) post tracing in of APOB^{-/-} organoids. Note at day 5, the extensive labelling of DNL-derived C16:1 species, which have a higher m/z due to the ¹³C (instead of ¹²C) labelling. Instead, no labelling for C20:4 is seen during the entire tracing period, as this fatty acid is essential and cannot be formed from DNL. f) Quantification of the percentage of glucose-driven DNL contribution for the five non-essential fatty acids that can be formed by DNL, after 1, 3, and 5 days of tracing in APOB^{-/-} organoids. The mean+SD from independent quantification in n=2 organoid cultures from 2 different donors per timepoint is shown.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds.-in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

### Organoids

The invention relates to human hepatocyte organoids comprising one or more modified genes. The term organoid is used to refer to self-organized three-dimensional tissue cultures. Organoids may include artificial, in vitro three-dimensional structures made to mimic or resemble the functional and/or histological structure of an organ or portion thereof, such as a liver organ.

Liver includes two types of epithelial cells hepatocytes and liver ductal cells. The organoids of the invention are derived from human hepatocyte cells. The hepatocytes may be primary hepatocytes. Primary hepatocytes are hepatocytes directly isolated from liver tissue. For example, hepatocytes obtained by biopsy. Primary hepatocytes are also commercially available from numerous suppliers. For example, from Axol Bioscience, Corning Inc., and Cytes Biotechnologies. Primary hepatocytes may be obtained from fetal, pediatric or adult livers.

Methods of forming hepatocyte organoids are known and described in, for example “Hendriks, D., Artegiani, B., Hu, H., Chuva de Sousa Lopes, S., and Clevers, H. (2021). Establishment of human fetal hepatocyte organoids and CRISPR-Cas9-based gene knockin and knockout in organoid cultures from human liver. Nature Protocols 16.” and “Hu, H., Gehart, H., Artegiani, B., LÖpez-Iglesias, C., Dekkers, F., Basak, O., van Es, J., Chuva de Sousa Lopes, S. M., Begthel, H., Korving, J., et al. (2018). Long-Term Expansion of Functional Mouse and Human Hepatocytes as 3D Organoids. Cell 175” both of which are expressly incorporated herein by reference. Further examples include the methods described in WO2020109324 and WO2019185017 which are expressly incorporated herein by reference.

In brief, hepatocyte organoids can be produced by first isolating primary hepatocytes from other cells. The hepatocytes are then cultured in cell culture medium which includes a scaffold that mimics the extracellular cellular matrix such as Matrigel. The scaffold provides a support the hepatocytes. The hepatocytes are then cultured in a culture medium and under conditions suitable for expansion of cells. Media is refreshed, for example every few days. The hepatocytes expand to form a 3-dimensional organoid. Once the hepatocytes have expanded to a particular number or size the cells are passaged (split) to form further cultures and thus further organoids.

In particular, organoids of the invention are derived from human hepatocyte tissue. That is to say that the organoids are not formed or derived from isolated stem cells.

### Modifications

The hepatocytes of the invention include modified genes. Modification refers to a change in the sequence of the gene, at the DNA level. Examples include insertions, mutations, substitution(s) and/or deletions. The modifications may result in the repression and/or complete absence of expression of a normal or “wild type” product encoded by the gene or in the increase in expression of a gene. Examples of such gene modifications are insertions, substitution(s), frameshift and missense mutations, deletions, knock-ins, or knock-outs of a gene or part of a gene, including deletions of the entire gene. Such modifications can occur in the coding region, e.g., in one or more exons, resulting either in the inability to produce a full-length product, functional product, or any product, such as by insertion of a stop codon, or resulting in changed protein function. Such modifications may also occur by modification in the noncoding regions, such as regulatory regions of gene. For example, modifications may be made at promoters or enhancers or other regions affecting activation of transcription, so as to prevent transcription of a gene. Gene disruptions include gene targeting, including targeted gene inactivation by homologous recombination (HR). Modifications may also include disruption of protein expression by targeting mRNAs. For example, by knock-downs. These modifications may lead to an increase or decrease in translation products (i.e. protein) produced from an mRNA transcript.

A “knockout” refers to the excision, inactivation or deletion of a gene within an organoid. This may involve, for example, introducing frameshift mutations using non-homologous end joining, or on the other hand changing the sequence of the DNA to introduce an exact STOP codon.

The term “knock-down” refers to suppression of the expression of a gene product, typically achieved by the use of antisense oligo deoxynucleotides and RNAi that specifically target the RNA product of the gene. Gene knock down refers to techniques by which the expression of one or more of an organoid's genes are reduced, either through genetic modification (a change in the DNA of one of the organism's chromosomes) or by treatment with a reagent such as a short DNA or RNA oligonucleotide with a sequence complementary to either an mRNA transcript or a gene.

Modifications may be homozygous or heterozygous modifications. “Heterozygous modification” refers to a modification in only one of two copies of a gene. For example, a heterozygous mutation can be a deletion, substitution, conversion, rearrangement, or insertion that functionally modifies one copy (allele) of the genes of the invention. A homozygous mutation refers to a mutation in both copies (alleles) of a gene.

The modifications described herein may lead to attenuation of the target gene. Attenuation refers to a reduction in the expression of the gene, a reduction in the amount of the expressed product of the gene (e.g. mRNA or translated protein) or a reduction in the activity of a product of the gene (e.g. reduced activity of a protein produced by the gene). reduction in the activity of a product of the gene may be due to one or more amino acid mutations, such as amino acid deletions, substitutions or insertions being introduced into the amino acid sequence of protein produced from the gene.

As such, the modifications described herein may result in reduced expression of a target gene. The terms “decrease,” “reduced,” “reduction,” and “decrease” are all used herein generally to mean a decrease by a statistically significant amount. For example, the decrease may be a decrease by at least 1% as compared to a reference level, for example a decrease by at least about 2%, or at least about 3%, or at least about 4%, or at least about 5%, or at least about 6%, or at least about 7%, or at least about 8%, or at least about 9%, or at least about 10%, or at least about 11%, or at least about 12%, or at least about 13%, or at least about 14%, or at least about 15%, or at least about 16%, or at least about 17%, or at least about 18%, or at least about 19%, or at least about 20%, or at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

Modifications described herein may result in increased expression of a target gene. The terms “increased”, “increase” or “enhance” or “activate” are all used herein to generally mean an increase by a statically significant amount, such as an increase of at least 1% as compared to a reference level, for example an increase of at least about 2%, or at least about 3%, or at least about 4%, or at least about 5%, or at least about 6%, or at least about 7%, or at least about 8%, or at least about 9%, or at least about 10%, or at least about 11%, or at least about 12%, or at least about 13%, or at least about 14%, or at least about 15%, or at least about 16%, or at least about 17%, or at least about 18%, or at least about 19%, or at least about 20%, or at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 0,1-fold, or at least about a 1-fold, or at least about a 2-fold, or at least about a 5-fold or at least about a 10-fold increase, or any increase between 0.1-fold and 10-fold or greater as compared to a reference level.

The reference level may be a level of expression of the target gene in a wild-type organoid or a control organoid. A control organoid may be an organoid that does not include any of the modified genes of the invention but may include other modifications.

Methods for determining changes in expression of genes are known. Non-limiting examples for methods of determining expression include, but are not limited to, RT-PCR, real time RT-PCR, next generation sequencing, western blot, dot blot, enzyme linked immunosorbent assay (ELISA). In some examples, the level of expression may be normalized to the expression of a house keeping gene.

Methods for modifying genes are well known in the art. For example, genes may edited in situ by way of gene editing techniques in order to provide a modified gene as described herein. Such genome editing and/or mutagenesis technologies are well known in the art. Particularly, the modification to the nucleic acid sequence is introduced by way of site-directed nuclease (SDN). The SDN may be selected from: meganuclease, zinc finger, transcription activator-like effector nucleases system (TALEN) or Clustered Regularly Interspaced Short Palindromic Repeats system (CRISPR) system. SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through nonhomologous end-joining (NHEJ) or homologous recombination (HR), resulting in targeted mutations (‘edits’). Particularly SDN may comprises techniques such as: Meganucleases, Zinc finger nucleases (ZFNs), Transcription Activator-Like Effector-based Nucleases (TALEN) (Feng et al. 2013 Cell Res. 23, 1229-1232, Sander & Joung Nat. Biotechnol. 32, 347-355 2014), and the Clustered Regularly Interspaced Short Palindromic Repeats (CRISPR-Cas) system. Gene editing may also be achieved by SDN-2. SDN-2 is similar to SDN, but also provides a small nucleotide template complementary to the area of the break. The template contains one or more sequence modifications to the genomic DNA which are incorporated to create the modification to the target gene. Preferably, the gene editing system may include a CRISPR-Cas system.

### CRISPR System

Embodiments of the present disclosure concern genome editing of hepatocyte organoids to edit or modify one or more genes using clustered regularly interspaced short palindromic repeats (CRISPR) and CRISPR-associated (Cas) proteins. See Sander and Joung, 2014.

In general, the term “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system may include a non-coding RNA molecule (guide) RNA, which sequence-specifically binds to DNA, and a Cas protein (e.g., Cas9), with nuclease functionality (e.g., two nuclease domains).

One or more elements of a CRISPR system may be derived from a type I, type II, or type III CRISPR system. One or more elements of a CRISPR system may be derived from a particular organism comprising an endogenous CRISPR system, such as Streptococcus pyogenes.

A Cas nuclease and gRNA (including a fusion of crRNA specific for the target sequence and fixed tracrRNA) are introduced into a cell of an organoid. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

The CRISPR system may induce DSBs at the target site, followed by modifications as discussed herein. Alternatively, Cas9 variants, deemed “nickases” may be used to nick a single strand at the target site. In some examples, paired nickases are used, e.g., to improve specificity, each directed by a pair of different gRNAs targeting sequences such that upon introduction of the nicks simultaneously, a 5′ overhang is introduced. In other examples, catalytically inactive Cas9 is fused to a heterologous effector domain such as a transcriptional repressor or activator, to affect gene expression.

In general, a CRISPR system is characterized by elements that promote the formation of a CRISPR complex at the site of a target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of a cell of an organoid. The target sequence may be within an organelle of a cell of an organoid. Generally, a sequence or template that may be used for recombination into the targeted locus comprising the target sequences is referred to as an “editing template” or “editing polynucleotide” or “editing sequence”. In some examples, an exogenous template polynucleotide may be referred to as an editing template. In some examples, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, the tracrRNA sequence, which may comprise or consist of all or a portion of a wild-type tracrRNA sequence (e.g., about or more than about 20, 26, 32, 45, 48, 54, 63, 67, 85, or more nucleotides of a wild-type tracrRNA sequence), may also form part of the CRISPR complex, such as by hybridization along at least a portion of the tracrRNA sequence to all or a portion of a tracr mate sequence that is operably linked to the guide sequence. The tracrRNA sequence has sufficient complementarity to a tracr mate sequence to hybridize and participate in formation of the CRISPR complex. As with the target sequence, complete complementarity is not necessarily needed.

One or more vectors driving expression of one or more elements of the CRISPR system are introduced into a cell of an organoid such that expression of the elements of the CRISPR system direct formation of the CRISPR complex at one or more target sites. For example, a Cas enzyme, a guide sequence linked to a tracr-mate sequence, and a tracrRNA sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracrRNA sequence embedded within one or more intron sequences (e.g., each in a different intron, two or more in at least one intron, or all in a single intron). In some examples, the CRISPR enzyme, guide sequence, tracr mate sequence, and tracr sequence are operably linked to and expressed from the same promoter.

A vector may include one or more insertion sites, such as a restriction endonuclease recognition sequence (also referred to as a “cloning site”). One or more insertion sites (may be located upstream and/or downstream of one or more sequence elements of one or more vectors. Following insertion of a guide sequence into the insertion site and upon expression the guide sequence directs sequence-specific binding of the CRISPR complex to a target sequence in a cell of an organoid.

A vector may include a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. The unmodified CRISPR enzyme may have DNA cleavage activity, such as Cas9.

The CRISPR enzyme directs cleavage of one or both strands at the location of a target sequence, such as within the target sequence and/or within the complement of the target sequence. A vector may encode a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). A Cas9 nickase may be used in combination with guide sequence(s), e.g., two guide sequences, which target respectively sense and antisense strands of the DNA target. This combination allows both strands to be nicked and used to induce NHEJ.

An enzyme coding sequence encoding the CRISPR enzyme may be codon optimized for expression in particular cells, such as eukaryotic cells. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid.

In general, a guide sequence is any polynucleotide sequence having sufficient complementarity with a target polynucleotide sequence to hybridize with the target sequence and direct sequence-specific binding of the CRISPR complex to the target sequence. The degree of complementarity between a guide sequence and its corresponding target sequence, when optimally aligned using a suitable alignment algorithm, may be about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

A guide sequence may be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to a cell having the corresponding target sequence, such as by transfection with vectors encoding the components of the CRISPR sequence, followed by an assessment of preferential cleavage within the target sequence, such as by Surveyor assay. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

As used herein, the term “guide RNA” or “gRNA” generally refers to an RNA molecule (or a group of RNA molecules collectively) that can bind to a CRISPR system effector, such as a Cas protein, and aid in targeting the Cas protein to a specific location within a target polynucleotide (e.g., a gene). A guide RNA of the invention can be an engineered, single RNA molecule (sgRNA), where for example the sgRNA comprises a crRNA segment and optionally a tracrRNA segment. A guide RNA of the invention can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Watson-Crick pairing of the sgRNA with the target site permits R-loop formation, which in conjunction with a functional PAM permits DNA cleavage or in the case of nuclease-deficient Cas9 allows tight binding to the DNA at that locus.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3′-overhang sequence. The

polynucleotide targeting guide sequence is a nucleic acid sequence that is complementary to a sequence in a target DNA. This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts.

As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention also provides a guide RNA comprising an engineered tracrRNA, wherein the tracrRNA further comprises a bait RNA segment that is capable of binding to a donor DNA molecule. The engineered tracrRNA may be a physically distinct molecule, as in a dual-guide system, or may be a segment of a sgRNA molecule.

The guide RNA, either as a sgRNA or as two or more RNA molecules, may not contain a tracrRNA, as it is known in the art that some CRISPR-associated nucleases, such as Cpfl (also known as Casl2a), do not require a tracrRNA for its RNA-mediated endonuclease activity (Qi et al., 2013, Cell, 152:1173-1183; Zetsche et al., 2015, Cell 163:759-771). Such a guide RNA may comprise a crRNA with the bait RNA operably linked at the 5′ or 3′ end of the crRNA. Cpfl also has RNase activity on its cognate pre-crRNA (Fonfara et al., 2016, Nature, doi.org/10.1038/natureI7945).

In general, a tracr mate sequence includes any sequence that has sufficient complementarity with a tracrRNA sequence to promote one or more of: (1) excision of a guide sequence flanked by tracr mate sequences in a cell containing the corresponding tracrRNA sequence; and (2) formation of a CRISPR complex at a target sequence, wherein the CRISPR complex comprises the tracr mate sequence hybridized to the tracrRNA sequence. In general, degree of complementarity is with reference to the optimal alignment of the tracr mate sequence and tracrRNA sequence, along the length of the shorter of the two sequences.

The CRISPR enzyme may be part of a fusion protein comprising one or more heterologous protein domains (e.g., about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags or reporter gene sequences.

A CRISPR enzyme in combination with (and optionally complexed with) a guide sequence may be delivered to a cell of an organoid. For example, CRISPR/Cas9 technology may be used to knock-out gene expression of the target gene in a cell of an organoid, knockout a gene or part thereof or knockin a polynucleotide sequence.

Gene modifications may also be carried out by prime editing. As used herein, “prime editing” refers to a genome editing system that directly writes new genetic information into a specified DNA site using a catalytically impaired nucleic acid-guided nuclease fused to a reverse transcriptase, programmed with a prime editing guide RNA (pegRNA) that both specifies the target site and encodes the desired edit (see. e.g., Anzalone et al., (Nature 574:464-465 (2019), which is incorporated herein by reference in its entirety). Genomic editing takes place by transfecting cells with the pegRNA and the fusion protein. Transfection is often accomplished by introducing vectors into a cell. Once internalized, the fusion protein nicks the target DNA sequence, exposing a 3′-hydroxyl group that can be used to initiate (prime) the reverse transcription of the reverse transcriptase template portion of the pegRNA. This results in a branched intermediate that contains two DNA flaps: a 3′ flap that contains the newly synthesized (edited) sequence, and a 5′ flap that contains the dispensable, unedited DNA sequence. The 5′ flap is then cleaved by structure-specific endonucleases or 5′ exonucleases. This process allows 3′ flap ligation, and creates a heteroduplex DNA composed of one edited strand and one unedited strand. The reannealed double stranded DNA contains nucleotide mismatches at the location where editing took place. In order to correct the mismatches, the cells exploit the intrinsic mismatch repair mechanism, with two possible outcomes: (i) the information in the edited strand is copied into the complementary strand, permanently installing the edit; (ii) the original nucleotides are re-incorporated into the edited strand, excluding the edit.

A nucleic acid encoding the guide RNA(s) and/or Cas9, is administered or introduced to a cell of an organoid. The nucleic acid typically is administered in the form of one or more expression vectors as described herein, such as a viral expression vector. In some examples, the expression vector is a retroviral expression vector, an adenoviral expression vector, a DNA plasmid expression vector, or an AAV expression vector. In some examples, one or more polynucleotides encoding the disruption system, such as the CRISPR based system, is delivered to a cell of an organoid. In some examples, the delivery is by delivery of one or more vectors, one or more transcripts thereof, and/or one or proteins transcribed therefrom.

One of skill in the art would be well-equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc), lentiviral vectors (e.g., derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors.

Methods for introducing vectors into cells are known and include, as non-limiting examples stable transformation methods, transient transformation methods, and virus mediated methods. The vectors may be introduced into the cell by for example, recombinant viral vectors (e.g., retroviruses, adenoviruses), liposome and the like. For example, transient transformation methods include microinjection, electroporation, or particle bombardment.

Vectors may also include selectable markers. The phrase “selectable marker” refers to a protein that enables the separation of cells expressing the marker from those that lack or do not express it. The selectable marker may be a fluorescent marker, for instance.

Expression of the marker by cells having successfully integrated the polynucleotide allows the isolation of these cells using methods such as, for example, FACS (fluorescent activated cell sorting). Alternatively, expression of a selectable marker may confer an advantageous property to the cell that allows survival of only those cells carrying the gene. For example, the marker protein may allow for the selection of the cell by conferring an antibiotic resistance to the cell. Consequently, when cells are cultured in medium containing said antibiotic, only cell clones expressing the marker protein that mediates antibiotic resistance are capable of propagating.

The presence of modifications in cells of an organoid may be confirmed by any known methods such as DNA sequencing methods, visual selection methods (for example based on visualisation of cells that have also been modified to include a reporter gene such as a fluorescent reporter gene), antibiotic selection methods (for example for cells that have also been modified to include an antibiotic marker gene) or RT-PCR.

Cells determined as containing the modified genes as described herein may then be isolated and expanded to produce organoids that include the modified genes.

### Genes

The genes that may be modified in organoids of the invention include at least one of APOB, MTTP, FADS2 and/or PNPLA3.

The APOB gene encodes Apolipoprotein B-100 (ApoB100) the human version of which is identified by the UniProt number P04114. The human APOB is identified as NCBI Gene ID: 338.

In human plasma, apoB occurs in two forms: apoB100 and apoB-48, which are both encoded by the APOB located on chromosome 2. ApoB-100 (the full-length translation product of apoB mRNA) is a large monomeric protein of 4536 amino acids that is synthesized in the liver. ApoB-100 is an essential component of liver-derived VLDLs, IDLs, and LDLs, where it serves as ligand for the LDL-receptor (LDL-R).

Apolipoprotein B-100 functions as a recognition signal for the cellular binding and internalization of LDL particles by the apoB/E receptor.

In view of the essential role of apoB100 in the assembly and secretion of apoB100 containing lipoproteins, apoB100 levels are regulated at multiple levels. Several factors influence the translocation process of newly synthesized apoB100. Most relevant is the availability of lipids at the site of apoB100 synthesis in the ER, which appears to dictate the amount of apoB100 secreted. In addition, the process of translocation is affected by the characteristics of apoB100 itself, including length, signal peptide polymorphism, and apoB100 folding to attain lipid-binding capability, which regulate its ability to assemble into lipoproteins. Successful transport and correct conformation of apoB100 may lead to its final secretion as a lipoprotein constituent. In the case of lipid shortage, nascent apoB100 translocation into the ER lumen is inefficient and domains of apoB100 are exposed to the cytosol, where newly synthesized apoB100 undergoes rapid intracellular degradation. The N-terminal assembly of lipids onto apoB100 during translocation requires the microsomal triglyceride transfer protein (MTTP). Heterozygous and homozygous mutations (which are described below) in APOB lead to disease such as familial hypobetalipoproteinemia (FHBL).

MTTP is an 894 amino acid protein located in the ER lumen that is a component of a protein complex involved in the early stages of apoB100 lipidation in liver. MTTP is encoded by the MTTP gene. The human MTTP gene is identified by the NCBI Gene number 4547 and the human MTTP protein is identified by UniProt ID Number P55157.

MTTP has been shown to bind to the first 1000 amino acids of apoB100 which form a domain capable of initiating nascent lipoprotein assembly (i.e., capable of recruiting lipids and facilitating the conversion of apoB100 into a buoyant lipoprotein particle). The physical interaction between apoB100 and MTTP is important for the initiation of translocation of the nascent apoB100 chain and for the co-translational addition of lipids to this chain. Via these mechanisms, MTTP is believed to avoid improper folding and premature degradation of apoB100.

The crucial role of MTTP in the assembly and secretion of apoB100 containing lipoproteins is substantiated by the observation that mutations in the MTTP gene, which abolish MTTP activity, are the cause of diseases such as abetalipoproteinemia (ABL), a severe recessive disorder in which VLDL and chylomicrons (CMs) are not secreted.

There is provided in one example, an human hepatocyte organoid that includes a modified APOB gene. In another example, there is provided a human hepatocyte organoid that includes a modified MTTP gene.

The organoids of the invention may include a modified FADS2 gene. The FADS2 gene encodes Fatty acid desaturase 2 (FADS2). FADS2 may also be referred to as Acyl-CoA 6-desaturase. The human FADS2 gene is identified by NCBI Gene ID: 9415 and the human FADS2 protein is identified by UniProt Number O95864.

FADS2 is involved in the biosynthesis of highly unsaturated fatty acids (HUFA) from the essential polyunsaturated fatty acids (PUFA) linoleic acid (LA) (18:2n-6) and alpha-linolenic acid (ALA) (18:3n-3) precursors, acting as a fatty acyl-coenzyme A (CoA) desaturase that introduces a cis double bond at carbon 6 of the fatty acyl chain. FADS2 catalyzes the first and rate limiting step in this pathway which is the desaturation of LA (18:2n-6) and ALA (18:3n-3) into gamma-linoleate (GLA) (18:3n-6) and stearidonate (18:4n-3), respectively. Subsequently, in the biosynthetic pathway of HUFA n-3 series, FADS2 desaturates tetracosapentaenoate (24:5n-3) to tetracosahexaenoate (24:6n-3), which is then converted to docosahexaenoate (DHA) (22:6n-3). FADS2 can also desaturate (11E)-octadecenoate (trans-vaccenoate, the predominant trans fatty acid in human milk) at carbon 6 generating (6Z,11E)-octadecadienoate and FADS2 exhibits Delta-8 activity with slight biases toward n-3 fatty acyl-CoA substrates.

FADS2 genetic polymorphisms are associated with fatty acid metabolism through for example changes in DNA methylation and gene expression.

The hepatocyte organoids of the invention may include a modified PNPLA3 gene. The PNPLA3 gene encodes patatin like phospholipase domain containing 3 (PNPLA3) protein. The human PNPLA3 gene is identified by NCBI Gene ID: 80339 and the human PNPLA3 protein is identified by the UniProt Number Q9NST1.

PNPLA3 catalyzes coenzyme A (CoA)-dependent acylation of 1-acyl-sn-glycerol 3-phosphate (2-lysophosphatidic acid/LPA) to generate phosphatidic acid (PA), an important metabolic intermediate and precursor for both triglycerides and glycerophospholipids.

Mutants and variants of PNPLA3 have been associated with accumulation of triglycerides in the liver. A mutation of isoleucine to methionine (I[ATC]>M[ATG]) SNP rs738409 has been confirmed to increase susceptibility to non-alcoholic liver disease (NAFLD) and also to have effects in diabetes. In some examples, the modification of PNPLA3 may be an I148M mutation.

In some examples organoids of the invention may include any combination of the modified genes described herein. For example, an organoid of the invention includes a modified Apolipoprotein B-100 (APOB) gene and a modified FADS2 gene. In one example an organoid of the invention includes a modified Apolipoprotein B-100 (APOB) gene and a modified PNPLA3 gene. In one example an organoid of the invention includes a modified Microsomal Triglyceride Transfer Protein (MTTP) gene and a modified FADS2 gene. In one example an organoid of the invention includes a modified Microsomal Triglyceride Transfer Protein (MTTP) gene and a modified PNPLA3 gene. In one example an organoid of the invention includes modified Apolipoprotein B-100 (APOB) gene, a modified FADS2 gene and a modified PNPLA3 gene. In one example an organoid of the invention includes a modified Microsomal Triglyceride Transfer Protein (MTTP) gene, a modified FADS2 gene and a modified PNPLA3 gene.

The modified genes of the invention are all involved in lipid homeostasis. Modifications of any of the genes described herein may lead to changes in lipid homeostasis in an organoid. Attenuating modifications of the genes described herein may lead to the accumulation of lipids in the cells of an organoid.

As such, the modifications described herein may lead to the formation of lipid droplets within the cells of the organoid. The area occupied by lipid droplets in organoids of the invention may be greater than the area occupied by lipid droplets in a wild-type or control hepatocyte organoid. That is to say, a greater area of an organoid of the invention may be occupied by lipid droplets in comparison to a wild-type or control organoid.

For example, one or more of the modifications described herein may lead to lipid droplets occupying an area of at least 1% of the total area of the organoid. For example at least 1%, 2%, 3%, 4%, 5%, 6%, 7%, 8%, 9%, 10%, 15%, 20%, 25%, 30%, 35%, 45%, 50%, 60%, 70%, 80%, 90% or more.

Lipid droplets may be detected and quantified using any known methods. For example, the lipids droplets may be labelled, for example using a fluorescence labelling moiety. The organoid may then be imaged using microscopy techniques such as fluorescent or confocal microscopy and the number of lipid droplets and area occupied by lipid droplets may be determined. For example, by manual inspection of the images of the organoid or by computer based software.

Without being bound by theory, the modifications described herein may lead to alterations in de novo lipogenesis. De novo lipogenesis refers to the process of synthesizing fatty acids (lipids) from acetyl-CoA subunits that are produced from a number of different pathways within the cell, most commonly carbohydrate catabolism. In brief, the reaction mechanism commences with the production of malonyl-CoA from an acetyl-CoA precursor, under the regulated catalytic activity of acetyl-CoA carboxylase (ACC). The malonyl-CoA is transferred to the prosthetic phosphopantetheine group of acyl carrier protein (ACP), a domain of the type I fatty acid synthase complex (FAS), with subsequent release of the coenzyme A carrier, catalysed by the activity of the malonyl/acetyl transferase (MAT) site of mammalian FAS. The prosthetic phosphopantetheine arm of ACP thus shuttles the elongating FA chain to the various catalytic centres in the active site cleft of FAS, aided by the rotation of FAS.

The ACP-bound malonyl moiety acts as the additive monomer for the elongation of the substrate acyl chain. Initially this is an acetyl unit bound to the thiol group of cysteine (Cys¹⁶¹) at the β-ketoacyl synthase (KS) active site. The malonyl moiety undergoes decarboxylative condensation with an acetyl moiety. ACP is then bound to a β-ketoacyl intermediate.

ACP shuttles the β-ketoacyl intermediate to the NADPH-dependent β-ketoreductase (KR) active site. The ketone of the β-carbon is reduced, generating a hydroxyl group. This is followed by sequential dehydration, by the dehydratase (DH) active site, and further reduction by NADPH-dependent enoyl-reductase (EnR). This generates a saturated acyl chain elongated by two carbon groups, which can act as the substrate for the next round of elongation as it binds the thiol-group of the cysteine at the catalytic site of KS.

The elongation ceases at the 16- or 18-carbon stage with release of palmitic acid or stearic acid from ACP via activity of the thioesterase (TE) domain of FAS. The specificity of FAS TE for 16-carbon acyl dictates the length of the FAs released in vitro as there is a rapid decline of TE activity for chain lengths less than 14-carbons, as it will not access the catalytic core of the domain, and greater than 18-carbons as it may not be accommodated by the binding groove of TE. The termination of chain elongation at this 16-carbon stage is further promoted due to acyl chains of this length or longer not readily transferring to the thiol-group of the active-site cysteine of KS. The incorporation of stable-isotope-labelled precursors into palmitate in humans, and tritium from ³H₂O in rat liver also supports palmitate as the major product of de novo lipogenesis.

While glucose is the main substrate for de novo lipogenesis, fructose is a highly lipogenic substrate and this is thought to arise from it bypassing the critical regulatory step catalysed by phosphofructokinase-1 (PFK-1) in glycolysis. Fructose is phosphorylated by fructokinase in the liver to fructose 1-phosphate (F1P). F1P is then the substrate for catalytic cleavage by aldolase, generating dihydroxy-acetone-phosphate (DHAP) and glyceraldehyde. Glyceraldehyde is subsequently phosphorylated by triokinase to produce glyceraldehyde 3-phosphate (G3P). Thus G3P and DHAP can enter glycolysis.

During de novo triacylglycerol (TG) synthesis fatty acids are incorporated through the initial acylation, by acyl-CoA, of glycerol-3-phosphate, generating lysophosphatidic acid (LPA). This step is catalysed by glycerol-phosphate acyl transferase (GPAT). LPA is further acylated by another acyl-CoA, under the catalysis of acylglycerol-phosphate acyl transferase (AGPAT), producing phosphatidic acid (PA). This is dephosphorylated by the action of phosphatidic acid phosphorylase (PAP) to generate diacylglycerol (DG). A final acyl-CoA is used to acylate the DG to a TG utilising the catalytic activity of diacylglycerol acyl transferase (DGAT).

As such, organoids of the invention may accumulate lipids via de novo lipogenesis. In particular, organoids including a modified APOB and/or MTTP gene may accumulate lipids, and therefore form lipid droplets, via do novo lipogenesis.

It has been found by the inventors that the modifications of the invention led to a downregulation of genes involved in de novo lipogenesis.

In particular, genes regulated by liver X receptors (LXR) may be downregulated. Liver X receptors α and β (LXRα and LXRβ) are nuclear receptors with roles in the transcriptional control of lipid metabolism. Transcriptional activity of LXRs is induced in response to elevated cellular levels of cholesterol. LXRs bind to and regulate the expression of genes that encode proteins involved in cholesterol absorption, transport, efflux, excretion and conversion to bile acids. The coordinated, tissue-specific actions of the LXR pathway maintain systemic cholesterol homeostasis and regulate immune and inflammatory responses. LXRs also regulate fatty acid metabolism by controlling the lipogenic transcription factor sterol regulatory element-binding protein 1c (SREBP-1c) and regulate genes that encode proteins involved in fatty acid elongation and desaturation. Endogenous agonists of LXRs include a variety of oxidized cholesterol derivatives referred to as oxysterols.

SREBP-1c (encoded by SREBF1) regulates genes involved in de novo lipogenesis, such as ACACA which encodes acetyl-CoA carboxylase (ACC), FASN which encodes fatty acid synthase (FAS) and the SCD gene that encodes stearoyl-CoA desaturase (SCD).

The organoids of the invention may therefore have downregulation of at least one of ACACA, FASN, DGAT2, SREBF1, HMGCS1, SQLE, LSS, and/or DHCR7.

HMGCS1 encodes Hydroxymethylglutaryl-CoA synthase (HMGCS1). HMGCS1 Catalyzes the condensation of acetyl-CoA with acetoacetyl-CoA to form HMG-COA, which is converted by HMG-COA reductase (HMGCR) into mevalonate, a precursor for cholesterol synthesis.

SQLE encodes squalene monooxygenase. Squalene monooxygenase Catalyzes the stereospecific oxidation of squalene to(S)-2,3-epoxysqualene, and is considered to be a rate-limiting enzyme in steroid biosynthesis.

LSS encodes lanosterol synthase. Lanosterol synthase is an oxidosqualene cyclase enzyme that converts-2,3-oxidosqualene to a protosterol cation and finally to lanosterol. Lanosterol is a key four-ringed intermediate in cholesterol biosynthesis.

DHCR7 encodes 7-dehydrocholesterol reductase. Delta-7-sterol reductase (EC 1.3.1.21), is the ultimate enzyme of mammalian sterol biosynthesis that converts 7-dehydrocholesterol (7-DHC) to cholesterol. This enzyme removes the C(7-8) double bond introduced by the sterol delta8-delta7 isomerases.

The organoids of the invention may further include lipids from exogenous sources. That is to say that the organoids may include lipids introduced into the organoid rather than produced within the organoid. Exogenous lipids may be introduced into an organoid via a process referred to as fat or lipid loading. For example, organoids may be cultured in culture medium that includes a high concentration of free fatty acids. The lipids may be dissolved in ethanol and conjugated to a carrier protein to aid entry into the cells of the organoid. For example, lipids may be conjugated to bovine serum albumin. Exogenous free fatty acids may be any free fatty acid for example, the exogenous lipids may include oleic acid (18:1) and/or palmitic acid (16:0).

Fat loading as described above may be used for providing a control organoid that ha similar or the same lipid accumulation as organoids of the invention. For example, a wild-type organoid or organoid not including the modified genes of the invention may be fat loaded. These control organoids may comparison of organoids of the invention and the mechanism of lipid accumulation and effects thereof to the control organoid.

### Uses

Organoids of the invention may be used as models for lipid homeostasis. The organoids may allow for the study of lipid homeostasis and discovery of agents that may alter lipid homeostasis. As organoids of the invention accumulate lipids the organoids may be useful for modelling of steatosis. Steatosis refers to the abnormal retention of fat (lipids) within a cell or organ. As such, the organoids of the invention may be used as models of liver steatosis. In particular, as described herein, the organoids of the invention may accumulate lipids via de novo lipogenesis. Therefore, organoids of the invention may be used as models of de novo lipogenesis steatosis. The organoids may be used as models of genetically caused steatosis. In addition or alternatively, the organoids may be used as models of diet driven steatosis and thus be used to find agents that may be helpful in subjects suffering from diet related steatosis.

The organoids of the invention may be used to help elucidate the genetic and enzymatic pathways involved in liver steatosis thus allowing providing information for possible new or previously unknown targets for reducing or preventing steatosis.

The organoids of the invention may also allow for the testing of agents for reducing steatosis in an ex vivo environment. For example, organoids of the invention may be exposed to agents and the level of lipid accumulation (steatosis) may be monitored and quantified. Thus, helping determine agents that may have a beneficial effect on steatosis in a clinical setting.

The organoids of the invention may also be used for modelling non-alcoholic fatty liver disease (NAFDL). As used herein, the term “non-alcoholic fatty liver disease” (NAFDL) refers to fatty liver cases in which there is no history of alcohol consumption or in which alcohol consumption is not related to the occurrence of liver steatosis. Fatty liver refers to a phenomenon in which there is abnormal accumulation of triglyceride in liver cells, compared to normal levels of triglyceride. About 5% of normal liver consists of fat tissue and the main components of the fat are triglycerides, fatty acids, phospholipids, cholesterols, and cholesterol esters. However, once the fatty liver occurs, most of the components are replaced with triglyceride. If the amount of triglycerides is more than 5% of the liver weight, it is diagnosed as fatty liver. The fatty liver may be caused by a lipid metabolism disorder or a defect in the process of carrying excessive fat in the liver cells, and is mainly caused by disorders of lipid metabolism in the liver. Non-alcoholic fatty liver disease may be categorized into primary and secondary non-alcoholic fatty liver diseases depending on the pathological cause. The primary one is caused by hyperlipidemia, diabetes, obesity or the like which is a characteristic of metabolic syndrome. The secondary one is a result of nutritional causes (sudden body weight loss, starvation, intestinal bypass surgery), various drugs, toxic substances (poisonous mushrooms, bacterial toxins), metabolic causes and other factors. Non-alcoholic fatty liver disease as used herein includes non-alcoholic fatty liver, non-alcoholic steatohepatitis (NASH), cirrhosis, and liver cancer.

The term “non-alcoholic steatohepatitis (NASH)” refers to a disease which occurs during the exacerbation process of non-alcoholic fatty liver disease (NAFLD), and in which triglyceride accumulates in the liver and the increase of Kupffer cells and the activation of phagocytes proceed in the fatty state. Subsequently, the oxidation of hepatocellular mitochondria occurs, causing inflammation and fibrosis. The main symptoms of the disease may include steatosis, inflammation, or ballooning in liver tissue, and may be accompanied by fibrosis of the liver tissue.

“Cirrhosis” refers to liver disease characterized by pathological loss of normal microscopic lobular architecture of the liver, fibrosis and nodular regeneration. Liver cirrhosis also refers to chronic interstitial inflammation of the liver.

As such, organoids of the invention may be used as models for NASH. Organoids of the invention may also be used as models for liver cancer. Also, organoids of the invention may be used as models of liver cirrhosis.

The organoids of the invention may also be used as models for diseases associated with the gene modifications described herein. For example, organoids of the invention that include at least a modified APOB gene may be used a models of hypobetalipoproteinaemia, in particular familial hypobetalipoproteinaemia.

Hypobetalipoproteinemias (HBLs) represent a heterogeneous group of disorders characterized by reduced plasma levels of total cholesterol (TC), low density lipoprotein-cholesterol (LDL-C) and apolipoprotein B (apoB) below the 5th percentile of the distribution in the population.

HBLs are defined as primary or secondary according to the underlying causes. Primary monogenic HBL are caused by mutations in several genes (APOB, PCSK9, MTP, SARA2). Familial hypobetalipoproteinemia (FHBL) is the most frequent monogenic form of HBL with a dominant mode of inheritance. It may be due to loss-of-function mutations in APOB.

Most APOB gene mutations reported cause the formation of premature termination codons in the apoB mRNA. The translation of these mRNAs leads to the formation of truncated apoBs of various size which, to a variable extent, lose the capacity to form plasma lipoproteins in liver and/or intestine and to export lipids from these organs.

Truncated apoBs may or may not be detectable in plasma according to their size. Truncated apoBs longer than apoB-29/30 (i.e., with a size corresponding to 29-30% of that of apoB-100, according to a centile nomenclature) are detectable in plasma (by immunoblot with an anti-apoB antibody), as they are secreted into the plasma as constituents of plasma lipoproteins. The detection of a truncated apoB in plasma suggests the presence of a mutation located in a genomic region spanning from exon 26 to exon 29 of APOB gene.

Truncated apoBs shorter than apoB-29/30, due to mutations located in the first 25 exons of APOB gene, are not detectable in plasma, as they are not secreted. These short truncated apoBs account for 30% of all APOB mutations reported so far in FHBL. Heterozygous FHBL subjects carrying truncated apoBs have a reduced production of apoB-containing lipoproteins in liver and, which prevents the formation of VLDL and CM, respectively. The production rate (in liver) of truncated apoBs, as compared with the corresponding wild-type forms of apoB (apoB-100), is greatly reduced for two main reasons: (i) the reduced lipid-binding capacity of structurally abnormal apoBs (notably short truncations) makes them prone to a rapid intracellular degradation, (ii) the presence of premature stop codons in apoB mRNAs due to frameshift or nonsense mutations may induce a rapid mRNA degradation (nonsense-mediated mRNA decay). In addition, FHBL carriers of long apoB truncations, which are secreted into the plasma, may have an increased removal of truncated apoB-containing lipoproteins by the liver (via the LDL-R) or by the kidney via megalin receptor.

Nonconservative amino acid substitutions in apoB have also been reported to be the cause of FHBL. For example, two mutations, R463W and L343V, were found to co-segregate with FHBL in two large Lebanese kindred. These mutations involve the N-terminal beta-alpha1 domain of apoB which contains sequence elements shown to be important for the proper folding of apoB. Other carriers of R463W have been identified in Italian, Dutch, and Spanish FHBL subjects, suggesting that R463W may be a recurrent mutation in the population. Other missense mutations have been reported to be the cause of FHBL. Five missense APOB mutations located within the N-terminal 1000 amino acids of apoB, namely A31P, G275S, L324M, G912D, and G945S, were identified in heterozygous carriers of FHBL in the Italian population. Among these mutations, the A31P substitution in apoB completely blocked apoB-48 secretion when expressed in rat hepatoma cells. In contrast to the two missense mutations L343V and R463W, the A31P mutant did not lead to ER retention as the aberrantly folded protein is degraded intracellularly by proteasomes and autophagosome/lysosome pathway.

In another example, organoids of the invention that include at least a modified MTTP gene may be used a models of abetalipoproteinemia (ABL).

The plasma lipid profile of ABL patients is characterized by extremely low plasma levels of TC, VLDL, and LDL and an almost complete absence of apoB-100 and apoB-48. ABL is due to mutations in the MTTP gene which is required for the assembly and secretion of apoB-100 containing lipoproteins in the liver. A variety of mutations in this gene, located on chromosome 4, have been described. Most of them result in truncated proteins devoid of function. Some MTP missense mutations have also been reported, which affect either the apoB-binding ability of MTP or its interaction with other components of the protein complex; they are associated with a milder form of the disease. It is conceivable that the severity of ABL phenotype is related to the residual activity of MTP and the capacity to form VLDL and CM. The absence of MTP activity leads to the accumulation of large lipid droplets in the cytoplasm in hepatocytes.

The organoids described herein may be used for drug discovery. For example, by exposing the organoids as described herein to agents and determining the effects of each agent on the phenotype of the organoids. For example, whether the agents have any effect on lipid homeostasis. For example effects on steatosis in the organoids. In some examples, the organoids may be used for CRISPR screening or CRISPR/Cas9 drug discovery screening. In some examples, the agents, drugs or genes may be mediators of lipid homeostasis. For example, Example 2 below shows the use of CRISPR screening to identify FADS2 as a target for mediating lipid homeostasis and ergo a target for treatment of steatosis and associated diseases such as NAFLD.

CRISPR screening refers to an experimental approach used to screen a population of mutant cells to discover genes involved in a specific phenotype. Instead of repressing genes at the post-transcriptional level, CRISPR introduces mutations to genes that render them non-functional.

Identifying genes that promote or reduce disease phenotypes may indicate possible targets for drug development. The simplest candidate drugs bind to and interfere with the proteins encoded by these genes, rather than affect the genes directly. CRISPR screening may help reveal more-subtle targets for drugs can be revealed by other methods by providing a better understanding of the importance of multiple genes and proteins, their interactions and their mutual regulatory effects. Many diseases, for example, arise when regulatory pathway that involve a complex network of intracellular interactions are mis-regulated. Using CRISPR screening to identify combinations of genes involved in these networks may offer a more in depth insight into possible drug targets.

CRISPR screening may be polled CRISPR screening or arrayed CRISPR screening. Pooled CRISPR screening is a powerful tool for identifying genes involved in biological mechanisms such as cell proliferation, drug resistance, and disease. Cells are transduced in bulk with a library of RNA guide-encoding vectors described herein, and the distribution of RNA guides is measured before and after applying a selective challenge. Pooled CRISPR screens work well for mechanisms that affect cell survival and proliferation, and they can be extended to measure the activity of individual genes (e.g., by using engineered reporter cell lines).

Arrayed CRISPR screens, in which only one gene is targeted at a time, make it possible to use RNA-seq as the readout. In some examples, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods, 2017 March; 14(3):297-301, which is incorporated herein by reference in its entirety.

### Medical Treatments

Using the liver organoids of the invention, the inventors have found that certain agents may be useful in treating a subject suffering from NAFDL having at least one modified gene as described herein. For example, a subject having at least one modified PNPLA3 gene; at least one modified FADS2 gene; at least one modified APOB gene; and/or at least one modified MTTP gene. For example a subject having at least one modified PNPLA3 gene. For example a subject having at least one modified FADS2 gene. For example a subject having at least one modified APOB gene. For example a subject having at least one modified MTTP gene.

The modifications may be a modification as described herein such as mutations that lead to attention of the gene or production of attenuated gene products (e.g. proteins that do not functional correctly or have reduced enzymatic activity).

Agents that may be for use in treating a subject or for use in methods of treating a subject having at least one modification as described herein include p38 inhibitors, FADS2 agonists, ACC inhibitors, DGAT2 inhibitors, FAS inhibitors, recombinant hFGF19 and/or FXR agonists.

p38 inhibitors refers to any molecule (e.g., small molecules or proteins) capable of inhibiting the activity of p38 family members. For example, as determined by Western blot quantification of phosphorylated p38 levels. p38 inhibitors include, for example, SB-203580 (4-(4-Fluorophenyl)-2-(4-methylsulfinylphenyl)-5-(4-pyridyl) 1H-imidazole), SB-239063 (trans-1-(4hydroxycyclohexyl)-4-(fluorophenyl)-5-(2-methoxy-pyrimidin-4-yl) imidazole), SB-220025 (5-(2amino-4-pyrimidinyl)-4-(4-fluorophenyl)-1-(4-piperidinyl) imidazole)), and ARRY-797.

Thus, provided herein is a p38 inhibitor for use in a method of treating NAFDL in subject as described herein.

Inhibition of p38 may lead to a reduction in steatosis. Without being bound by theory inhibition of p38 may alter hepatic p38 signaling which has been implicated in regulating gluconeogenesis.

FADS2 agonists refers to any agent, such as a small molecule, nucleic acid or protein that increases the activity of fatty acid desaturase 2. Such molecules may directly activate the FADS2 protein or may directly indirectly target a FADS2 encoding gene. For example, FADS2 agonists may target transcriptional regulators of a FADS2 gene in order to increase expression and production of FADS2. FADS2 agonists also includes endogenous and/or exogenous nucleic acids or proteins that may be introduced into a subject and express or provide an endogenous and/or exogenous FADS2.

In some examples, the activity of FADS2 is increased in the liver of the subject. In particular, the FADS2 activity may be increased in hepatocytes of the subject.

For example, the agent may be a nucleic acid or polypeptide encoding a human FADS2 (for example as identified by the UniProtKB number 095864). In some examples, the nucleic acid may be part of an expression vector, such as a viral expression vector.

In some examples, the expression vector is a retroviral expression vector, an adenoviral expression vector, a DNA plasmid expression vector, or an AAV expression vector. In some examples, one or more polynucleotides encoding the endogenous and/or exogenous FADS2, such as a human FADS2, is delivered to a cell of the subject. In some examples, the delivery is by delivery of one or more vectors, one or more transcripts thereof, and/or one or proteins transcribed therefrom.

In some examples, the FADS2 agonist may be an agent, such as a small molecule, nucleic acid or polypeptide that increases the activity of one or more genes or polypeptides that encode transcription factors or other modulators of FADS2 that may act to increase activity (e.g. expression and/or enzymatic activity) of FADS2.

In some examples, the agent may be delivered to a liver of the subject. In some examples, the agent may be delivered to hepatocytes of the subject.

Thus, there is provided a FADS2 agonist for use in a method treating NAFLD in a subject as described herein.

There is also provided a FADS2 agonist for use in treating NAFDL; for use in reducing and/or preventing steatosis; and/or for use in treating or preventing a cardiovascular disease in a subject in need thereof. The subject may not include any of the gene modifications described herein. In other examples, the subject may have one or more of the gene modifications described herein. For example, the subject may suffer from a monogenic lipid disorder. For example, familial hypobetalipoproteinaemia (FHBL) and abetalipoproteinemia (ABL).

Without being bound by theory it has been shown that FADS2 may limit the degree of steatosis development in APOB−/− or MTTP−/− organoids and as such in subjects having the same mutations. Furthermore, loss of FADS2 induces spontaneous lipid accumulation in wild type organoids. SNPs in FADS2 have been linked with liver function (Chambers et al., 2011), and very recently with NAFLD (Vujkovic et al. bioRxiv which is incorporated herein by reference). Collectively, these indications point toward a role for FADS2, and as such PUFA metabolism, in NAFLD, highlighting FADS2 as a NAFLD target.

There is also provided methods of reducing and/or preventing steatosis in a subject reducing and/or preventing steatosis in a subject in need thereof, comprising increasing FADS2 activity in the subject. There is also provided methods of treating or preventing a cardiovascular disease in a subject in need thereof, comprising increasing FADS2 activity in the subject. There is also provided methods treating or preventing a cardiovascular disease in a subject in need thereof, comprising increasing FADS2 activity in the subject.

The term ‘cardiovascular disease’ refers to diseases affecting the heart or blood vessels or both. In particular, cardiovascular disease includes arrhythmia (atrial or ventricular or both); atherosclerosis and its sequelae; angina; cardiac rhythm disturbances; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart, kidney or other organ or tissue; endotoxic, surgical, or traumatic shock; hypertension, valvular heart disease, heart failure, abnormal blood pressure; vasoconstriction (including that associated with migraines); vascular abnormality, inflammation, insufficiency limited to a single organ or tissue.

In some examples, exogenous FADS2 may refer to FADS2 derived from other organism other than humans. For example, from other animals such as a mouse, monkey or porcine. In some examples, exogenous FADS2 may refer to a variant of human FADS2. For example, a human FADS2 including one more mutations, modifications or additional elements. For example, to help or aid in delivery, expression or action of the FADS2 in the subject.

Changes, such as an increase in the activity or an amount of FADS2 may be detected in a subject by any known means and may depend on how FADS2 activity is increased (e.g. the FADS2 agonist administered). For example, increased expression of FADS2 may be determined by methods such as RT-PCR. Amounts of FADS2 proteins or polypeptides in a subject may be determined by methods such as enzyme-linked immunosorbent techniques, absorbance based methods, biuret test derived assays, spectrometry methods, antibody based techniques, western blot analysis methods, immunoelectrophoretic methods, immunoprecipitation based assays or any other methods known in the art. Changes, such an increase in FADS2 activity, may be determined by any known methods in the art such as enzymatic assays. The change in the amount FADS2 or FADS2 activity in a subject may be determined in comparison to a control amount or activity. For example, in comparison to a control amount or activity determined from the amount or activity in a healthy subject. In some examples, the control amount or activity may be determined based on the amount or activity in a subject as described herein but has not been administered a FADS2 agonist as described herein.

In some examples, increasing FADS2 activity in a subject may increase the amount of triacylglycerides having longer carbon chains. For example, increasing the level of triacylglycerides having at least a 54 carbon backbone or chain. In some examples, increasing the activity of FADS2 in a subject may increase the degree of unsaturation of triacylglycerides in a subject. For examples, increased FADS2 activity may decrease the number of triacylglycerides comprising at least one saturated fatty acid in a subject. In some examples, increasing the activity of FADS2 may decrease the amount of fatty acids in a subject. In some examples, increasing the level of FADS2 may decrease the de novo lipogenesis (DNL) index in a subject.

DNL index refers to the ratio of the endogenously produced palmitic acid (16:0), the main product of DNL, and the essential FA linoleic acid (18:2n-6) whose origin is from dietary lipids. The DNL-index has may be used as a tool to assess fatty acid synthesis in a subject.

In some examples, the steatosis may be dietary driven steatosis. That is to say that the subject may suffer from or be at risk of steatosis caused by the subjects diet. For example, a diet comprising high levels of saturated fats. For example, a diet comprising meats such as red meats and poultry, dairy products, baked goods, confectionary, and foods high in sugars or comprising added sugars.

In some examples, steatosis may be linked with metabolic disorders such as diabetes, insulin resistance, high blood pressure, cholesterolemia, dyslipidemia, high fatty acids or metabolic syndrome.

In some examples, steatosis may be genetically driven. For example, by one of the genetic modifications described herein.

In some examples, steatosis may be caused by medications. For example, amiodarone, diltiazem, tamoxifen or steroids.

Steatosis and NAFLD may be diagnosed by any known methods in the field. For example, using methods such as ultrasound, computed tomography, liver biopsy, enzymatic tests, magnetic resonance scanning or the like.

In some examples, increasing activity of FADS2 and use of a FADS2 agonist may prevent NASH and/or cirrhosis. Without being bound by theory, by reducing steatosis in a subject, this may prevent NAFLD developing into NASH and therefore may reduce or prevent permanent damage to the liver of a subject. As such, there is also provided a method of preventing and/or reducing the risk of NASH and/or cirrhosis in a subject by increasing FADS2 in a subject as described herein.

In some examples, the FADS2 agonist may include or b a gene editing system. For example, a gene editing system such as a CRISPR system as described herein which targets a FADS2 gene of the subject or genes encoding transcription factors or other modulators of FADS2 that may act to increase activity (e.g. expression and/or enzymatic activity) of FADS2.

ACC inhibitor refers to any therapeutic agent that reduces the activity of the acetyl-CoA carboxylase enzyme. Exemplary ACC inhibitors include 4-(4-[(1-isopropyl-7-oxo-1,4,6,7-tetrahydro-TH-spiro [indazole-5,4′-piperidin]-1′-yl) carbonyl]-6-methoxypyridin-2-yl)benzoic acid, gemcabene, and firsocostat (GS-0976) and pharmaceutically acceptable salts thereof.

Thus, there is provided an ACC inhibitor for use in a method treating NAFLD in a subject as described herein.

DGAT2 inhibitors refers to any agent that inhibit or reduce Diacylglycerol O-Acyltransferase 2 activity or transcription.

Examples of DGAT2 inhibitor are a polymethoxylated flavone (PMF) such as polymethoxylated, mono-methoxylated flavones and/or hydroxylated flavones. For example, tangeretin, nobiletin, and citrus flavonoids. Other suitable PMF include limocitrin, limocitrin derivatives, quercetin and quercetin derivatives, including, but not limited to, limocitrin-3,7,4′-trimethylether (5-hydroxy-3,7,8,3′,4′-pentamethoxyfiavone); limocitrin-3,5,7,4′-tetramethylether (3,5,7,8,3′,4′-hexamethoxyflavone); limocitrin-3,5,7,4′-tetraethylether (8,31-dimethoxy-3,5,7,4′-hexamethoxyflavone); limocitrin-3,7,4′-trimethylether-5-acetate; quercetin tetramethylether (5-hydroxy-3,7,3′,4′-tetramethoxyflavone); quercetin-3,5-dimethylether-7,3′,4′-tribenzyl ether; quercetin pentamethyl ether (3,5,7,3′,4′-pentamethoxyflavone); quercetin-5,7,3′,4′-tetramethylether-3-acetate; and quercetin-5,7,3′,4′-tetramethylether (3-hydroxy-5,7,3′,4′-tetramethoxyflavone); and the naturally occurring polymethoxyflavones: 3,5,6,7,8,3′,4′-heptan-ethoxyflavone; 5-desmethylnobiletin (5-hydroxy-6,7,8,3′,4′-pentamethoxyflavone); tetra-O-methylisoscutellarein (5,7,8,4′-tetramethoxyflavone); 5-desmethylsinensetin (5-hydroxy-6,7,3′,4′-tetramethoxyflavone); and sinensetin (5,6,7,3′,4′-pentamethoxyflavone). Another suitable PMF is tocotrienol. Further details on compositions that inhibit DGAT2 can be found in US Pat Pub No. 2008/0166420, WO2006/132879, and Gangi et al. (2004) J. Lipid Res. 45:1835-1845 the disclosures of which are incorporated herein by reference.

DGAT2 inhibitor may be administered in combination with a separate DGAT1 inhibitor. DGAT1 inhibitors include Pradigastat A922500, T863, AZD-7687, or AZD 3988. The DGAT1 and DGAT2 inhibitors can be given simultaneously or sequentially, such as within 24 or 48 hours. Other suitable DGAT1 inhibitors include the DGAT1 inhibitors described in: WO04047755, W00204682, W09745439, US20030154504, US20030167483, WO9967403, W09967268, W005013907, W005044250, W006064189, W006004200, W006019020, US20040209838, US20040185559, WO04047755, US20040224997, W005072740, JP2006045209, WO06044775, JP2004067635, JP2005206492, U.S. Pat. No. 6,100,077, W004100881, WO06113919, W0072740, WO09126624, WO022551, and WO07141545, their salts or esters, etc. These patents and publications are incorporated by reference herein in their entireties, and in particular for their disclosure of DGAT1 inhibitors.

Thus, there is provided a DGAT2 inhibitor for use in a method treating NAFLD in a subject as described herein. In some examples, there is provided the use of a DGAT2 and DGAT1 inhibitor for use in a method treating NAFLD in a subject as described herein.

Without being bound by theory DGAT2-silenced organoids were viable and fat-free.

FAS inhibitors reefers to agents that inhibit fatty acid synthase (encoded by the FASN gene). Fatty acid synthase is a multi-enzyme protein that catalyzes fatty acid synthesis. It is not a single enzyme but a whole enzymatic system composed of two identical 272 kDa multifunctional polypeptides, in which substrates are handed from one functional domain to the next. Its main function is to catalyze the synthesis of palmitate (C16:0, a long-chain saturated fatty acid) from acetyl-CoA and malonyl-CoA, in the presence of NADPH. Fatty acids are synthesized by a series of decarboxylative Claisen condensation reactions from acetyl-CoA and malonyl-CoA. Following each round of elongation the beta keto group is reduced to the fully saturated carbon chain by the sequential action of a ketoreductase (KR), dehydratase (DH), and enoyl reductase (ER). The growing fatty acid chain is carried between these active sites while attached covalently to the phosphopantetheine prosthetic group of an acyl carrier protein (ACP), and is released by the action of a thioesterase (TE) upon reaching a carbon chain length of 16 (palmitic acid).

Examples of inhibitors include, but is not limited to TVB-2640; TVB-3664; TVB-3166; TVB-3150; TVB-3199; TVB-3693; BZL-101; 2-octadecynoic acid; MDX-2; Fasnall; MT-061; G28UCM; MG-28; HS-160; GSK-2194069; KD-023; or cilostazol.

Without being bound by theory, inhibition of FAS may lead to downregulation of several key genes involved in glycolysis (ALDOB, HK2, ENO2, PFKFB3/4).

Thus, there is provided a FAS inhibitor for use in a method treating NAFLD in a subject as described herein.

FGF19 is a member of the most distant of the seven subfamilies of the FGFs. FGF19 is a high affinity ligand of FGFR4 (Xie et al (1999) Cytokine 11:729-735). FGF19 is normally secreted by the biliary and intestinal epithelium. FGF19 plays a role in cholesterol homeostasis by repressing hepatic expression of cholesterol-7-α-hydroxylase 1 (Cyp7α1), the rate-limiting enzyme for cholesterol and bile acid synthesis. recombinant hFGF19 refers to recombinant human or humanized FGF19. For example, as described in U.S. Pat. No. 8,409,579B2 and PCT International Publication WO2021092140A1 which are incorporated herein by reference.

Without being bound by theory, hFGF19 may repress various de novo lipogenesis-related genes, including DGAT2, ACSS2, and GPAM, leading to a reduction in steatosis.

Thus, there is provided a hFGF19 for use in a method treating NAFLD in a subject as described herein.

FXR agonists refer to any agent that is capable of binding and activating farnesoid X receptor (FXR) which may be referred to as bile acid receptor (BAR) or NR1H4 (nuclear receptor subfamily 1, group H, member 4) receptor. FXR agonists may act as agonists or partial agonists of FXR. The agent may be a chemical compound or biological molecule (e.g., a protein or antibody). The activity of a FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

Exemplary FXR agonists include, but are not limited to obeticholic acid (OCA), GS-9674, LJN-452 or LJN452, LMB763, EDP-305, AKN-083, INT-767, GNF-5120, LY2562175, INV-33, NTX-023-1, EP-024297, Px-103 and SR-45023. Other examples include GW4064 (as described in PCT Publication No. WO 00/37077 or in US 2007/0015796), 6-ethylhenodeoxycholic acid (6ECDCA), in particular 3a, 7a-dihydroxy-6a-ethyl-5b-holan-24-ovic acid, also known as INT-747; 6 ethylucrystal, deoxycholate, taurocholate, taurodesoxycholate, chenodeoxycholic acid, 7v-methylcholic acid, and methyllithocholic acid.

Without being bound by theory, FXR agonist may have similar effects as those seen for hFGF19. In addition, FXR agonists may strongly upregulate endogenous FGF19 expression. Therefore, FXR agonists may not only stimulates secretion of FGF19 from the gut to the liver, but also exert the same function directly in the liver. Furthermore, typical bile acid synthesis-related FXR target genes such as CYP7A1 and CYP27A1 may be downregulated and NR0B2 (SHP) may be upregulated by FXR agonists.

Thus, there is provided a FXR agonist for use in a method treating NAFLD in a subject as described herein.

As described herein, a subject may include at least one modified APOB gene and may suffer from FHBL. Thus, provided herein are p38 inhibitors, FADS2 agonists, ACC inhibitors, DGAT2 inhibitors, FAS inhibitors, recombinant hFGF19 and/or FXR agonists for use in methods of treating FHBL. For example, there is provided p38 inhibitors for use in methods of treating FHBL. For example, there is provided FADS2 agonists, for use in methods of treating FHBL. For example, there is provided ACC inhibitors, for use in methods of treating FHBL. For example, there is provided DGAT2 inhibitors, for use in methods of treating FHBL. For example, there is provided FAS inhibitors, for use in methods of treating FHBL. For example, there is provided recombinant hFGF19, for use in methods of treating FHBL. For example, there is provided FXR agonists, for use in methods of treating FHBL. In particular the FHBL is associated with an apoB-100 mutation, truncation, reduced activity or loss of function.

As described herein, a subject may include at least one MTTP gene modification as described and may suffer from ABL.

Thus, provided herein are p38 inhibitors, FADS2 agonists, ACC inhibitors, DGAT2 inhibitors, FAS inhibitors, recombinant hFGF19 and/or FXR agonists for use in methods of treating ABL. For example, there is provided p38 inhibitors for use in methods of treating ABL. For example, there is provided FADS2 agonists, for use in methods of treating ABL. For example, there is provided ACC inhibitors, for use in methods of treating ABL. For example, there is provided DGAT2 inhibitors, for use in methods of treating ABL. For example, there is provided FAS inhibitors, for use in methods of treating ABL. For example, there is provided recombinant hFGF19, for use in methods of treating ABL. For example, there is provided FXR agonists, for use in methods of treating ABL.

Subjects that include at least one of the modifications as described herein may suffer from NASH. Thus, provided herein are p38 inhibitors, FADS2 agonists, ACC inhibitors, DGAT2 inhibitors, FAS inhibitors, recombinant hFGF19 and/or FXR agonists for use in methods of treating NASH. For example, there is provided p38 inhibitors for use in methods of treating NASH. For example, there is provided FADS2 agonists, for use in methods of treating NASH. For example, there is provided ACC inhibitors, for use in methods of treating NASH. For example, there is provided DGAT2 inhibitors, for use in methods of treating NASH. For example, there is provided FAS inhibitors, for use in methods of treating NASH. For example, there is provided recombinant hFGF19, for use in methods of treating NASH. For example, there is provided FXR agonists, for use in methods of treating NASH.

In some examples, the subject may include a modified PNPLA3. The modification may be homozygous or heterozygous PNPLA3 I148M mutant. In other examples, the subject may include a modified FADS2 that includes a single nucleotide polymorphism such as rs3834458 and rs66698963.

Provided herein is also a method of reducing steatosis in hepatocytes in a subject as described herein. The method includes administering an agent that targets de novo lipogenesis. For example, administering at least one of a p38 inhibitor as described herein; FADS2 agonist as described herein; ACC inhibitor as described herein; FXR agonist as described herein; FAS inhibitor as described herein; DGAT2 inhibitor as described herein; and/or recombinant hFGF19 as described herein.

In particular the subject may suffer from NAFLD. In some examples the subject may suffer from NASH. In some examples the subject may suffer from liver cancer. In some examples the subject may include a modified APOB gene as described herein and suffer from FHBL. In some examples, the subject may include a modified MTTP gene as described herein and suffer from ABL.

Without being bound by theory, the inventors have found that any one or more of the agents described herein lead to indication of DUSP4 and DUSP5. DUSPs (dual-specificity phosphatases) regulate MAPK signaling pathway activity, including ERK, JNK, and p38. DUSP4 encodes for Dual specificity protein phosphatase 4 (DUSP4) which is identified by UniProtKB number Q13115. DUSP4 regulates mitogenic signal transduction by dephosphorylating both Thr and Tyr residues on MAP kinases ERK1 and ERK2.

DUSP5 encodes Dual specificity protein phosphatase 5 (DUSP5) which is identified by UniProtKB number Q16690. DUSP5 is active with phosphotyrosine, phosphoserine and phosphothreonine residues. The highest relative activity is toward ERK1.

Thus, provided herein is a method of treating NAFLD by inducing DUSP4 and/or DUSP5 in a subject as described herein. In particular, the method may include administering an agent that inhibits p38 signaling. For example, a p38 inhibitor as described herein.

The agents described herein may be formulated as pharmaceutical compositions. A pharmaceutical composition may include at least one pharmaceutically acceptable excipient, i.e., one or more pharmaceutically acceptable carrier substances and/or additives, e.g., buffers, carriers, excipients, stabilizers, etc. The pharmaceutical composition may be provided in the form of a kit.

Relative amounts of the active ingredient (e.g. at least one of a p38 inhibitor as described herein; FADS2 agonist as described herein; ACC inhibitor as described herein; FXR agonist as described herein; FAS inhibitor as described herein; DGAT2 inhibitor as described herein; and/or recombinant hFGF19 as described herein), a pharmaceutically acceptable excipient, and/or any additional ingredients in a pharmaceutical composition may vary, depending upon the identity, size, and/or condition of the subject being treated and further depending upon the route by which the composition is to be administered.

For example, the composition may comprise between 0.1 percent and 99 percent (w/w) of the active ingredient. By way of example, the composition may comprise between 0.1 percent and 100 percent, e.g., between 0.5 and 50 percent, between 1-30 percent, between 5-80 percent, at least 80 percent (w/w) active ingredient.

The pharmaceutical compositions can be formulated using one or more excipients or diluents to (1) increase stability; (2) permit the sustained or delayed release of the payload; (3) alter the biodistribution; and/or (6) alter the release profile.

A pharmaceutically acceptable excipient may be at least 95 percent, at least 96 percent, at least 97 percent, at least 98 percent, at least 99 percent, or 100 percent pure. An excipient may be approved for use for humans and for veterinary use. An excipient may be approved by United States Food and Drug Administration. An excipient may be of pharmaceutical grade. An excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, MD, 2006; incorporated herein by reference in its entirety). The use of a conventional excipient medium may be used, except insofar as any conventional excipient medium may be incompatible with a substance or its derivatives, such as by producing any undesirable biological effect or otherwise interacting in a deleterious manner with any other component(s) of the pharmaceutical composition.

The agents or pharmaceutical compositions thereof described herein may be administered to a subject in at a therapeutically effective amount. A “therapeutically effective amount” means a dose or plasma concentration in a subject that provides the desired specific pharmacological effect, e.g. to reduce steatosis in the liver. The therapeutically effective amount may vary based on the route of administration and dosage form, the age and weight of the subject, and/or the disease or condition being treated.

The agents or pharmaceutical compositions thereof described herein may be administered by oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver) or other parenteral route depending on the desired route of administration and the tissue that is being targeted.

The timing of administration can vary from individual to individual, depending upon such factors as the severity of an individual's symptoms. For example, an effective dose of the agents or compositions described herein can be administered to an individual once every six months for an indefinite period of time, or until the individual no longer requires therapy. A person of ordinary skill in the art will recognize that the condition of the individual can be monitored throughout the course of treatment and that the effective amount of agents or compositions described herein that is administered can be adjusted accordingly.

Agents or compositions described herein may be for use in combination with one or more other therapeutic, prophylactic, research or diagnostic agents. By “in combination with,” it is not intended to imply that the agents must be administered at the same time and/or formulated for delivery together. Agents or compositions described herein can be administered concurrently with, prior to, or subsequent to, one or more other desired therapeutics or medical procedures. The effect of the two treatments can be partially additive, wholly additive, or greater than additive.

The delivery can be such that an effect of the first treatment delivered is still detectable when the second is delivered. The agents or compositions described herein and the at least one additional therapy can be administered simultaneously, in the same or in separate compositions, or sequentially.

Dosages of the agents or compositions described herein to be administered to a subject depend upon the mode of administration, the severity of steatosis to be treated and/or prevented, the individual subject's condition, the individual subject's risk (such as genetic risk), and can be determined in a routine manner.

### EXAMPLES

### Methods

### Organoid Culture

The use of human fetal livers for research was approved by the Dutch Ethical Medical Council (Leiden University Medical Center). Human hepatocyte organoid lines were established and cultured in HEP medium as described in (Hendriks et al., 2021; Hu et al., 2018). In brief, hepatocytes were isolated by collagenase type IV digestion and enriched for using low-speed centrifugations. The hepatocytes were washed with AdvDMEM+++ (AdDMEM/F12 medium supplemented with 1× GlutaMAX, 10 mM HEPES, and 100 U/ml penicillin-streptomycin solution) and seeded in 100 μl BME suspension (2:1 BME:AdvDMEM+++) per well of a 12-well plate (3 droplets/well). Organoids were maintained in HEP medium (AdvDMEM+++ supplemented with 15% RSPO1-conditioned medium, 1× B-27 Supplement Minus Vitamin A, 2.5 mM nicotinamide, 1.25 mM N-acetyl-L-cysteine, 50 ng/ml EGF, 50 ng/ml FGF7, 50 ng/ml FGF10, 50 ng/ml HGF, 20 ng/ml TGFα, 10 nM gastrin, 3 μM CHIR-99021, 1 μM A 83-01, 5 μM Y-27632, and 50 μg/ml primocin). Organoids were typically passaged every 7-14 days at a 1:2-1:4 ratio by manual pipetting with a P1000 pipette. During the first few days of organoid line establishment and upon passaging of the organoids HEP medium was supplemented with extra Y-27632 (final concentration: 10 μM) to minimize anoikis.

### CRISPR Gene Knock-Out

Organoids were CRISPR-engineered using Cas9/NHEJ-mediated gene disruption. Single-guide RNAs were designed using an online web-tool (www.alum.bio/eCommerce/cas9/ input) and cloned into the pSPgRNA plasmid (Addgene #47108) as described previously (Ran et al., 2013). The sgRNA sequences of targeted genes are given in Table 1.

TABLE 1A
Gene Knock-out

| | | SEQ |
|---|---|---|
| | | ID |
| Plasmid name | Spacer sequence | NO |
| ACACA-KO-sgRNA | CTTCCCTAATCTCTTCAGAC | 1 |
| ACACB-KO-sgRNA | GACAGATTTCTTACACTCCC | 2 |
| ACE2-KO-sgRNA | AGAACAGGTCTTCGGCTTCG | 3 |
| ACSL1-KO-sgRNA | GAAGAGTACGCACGTACTGT | 4 |
| AKR1C1-KO-sgRNA | TGTTACCTCTGCAGGCGCAT | 5 |
| AKR1C2-KO-sgRNA | ATCATTCAGCTTCACACAC | 6 |
| APOB-KO-sgRNA | CAGCCAGTGCACCCTGAAAG | 7 |
| BHLHE40-KO-sgRNA | GAGCCGGTGCGGCAATTTGT | 8 |
| DGAT2-KO-sgRNA | GACCTGCGCTGTCGCGCGAG | 9 |
| FADS2-KO-sgRNA | CCAGACTTACGTTCTTGCCG | 10 |
| FASN-KO-sgRNA | GGACAACCTCATCGGCGGTG | 11 |
| LPCAT1-KO-sgRNA | CGCACCATGTGGTTCGCCGG | 12 |
| LSS-KO-sgRNA | GAACGGGATGACATTTTACG | 13 |
| MOGAT3-KO-sgRNA | GAAAGTGAGCACATATTGGT | 14 |
| MTTP-KO-sgRNA | TGACCAGTTGATCCAAATAA | 15 |
| MVD-KO-sgRNA | GTGCAGAGTGACGCTCAGGG | 16 |
| PCKS9-KO-sgRNA | GGTGCTAGCCTTGCGTTCCG | 17 |
| PNPLA3-KO-sgRNA | GCGCATGTTGTTCGGCGCTT | 18 |
| PLIN1-KO-sgRNA | CAACAAAGGCCTCACCTTGC | 19 |
| SCARB1-KO-sgRNA | CCTGCGGCTTCTCGCCCTTC | 20 |
| SERPINE1-KO-sgRNA | GAAAACCACGTTGCGGTCCT | 21 |
| SLC29A3-KO-sgRNA | AATAGTGGCCGTTGTCTCAG | 22 |
| STARD8-KO-sgRNA | ATGAACCTCCAGTTTCATCG | 23 |
| TRIB3-KO-sgRNA | GCCCACTTCGAGCTCGTTTC | 24 |

TABLE 1B
Gene Knock-in

| | | SEQ |
|---|---|---|
| | | ID |
| Plasmid name | Spacer sequence | NO |
| PLIN2-KI-sgRNA | GAGTTTTATGCTCAGATCGC | 25 |

TABLE 1C
Prime Editing

| Plasmid | | |
|---|---|---|
| name | Spacer sequence | 3'-extension |
| PNPLA3- | GGATAAGGCCACTGTAGAAG | GCTTCATGCCCTTC |
| 1148M- | (SEQ ID NO: 26) | TACAGTGGCCTTA |
| pegRNA | | (SEQ ID NO: 29) |
| PNPLA3- | GGATAAGGCCACTGTAGAAG | GCTTCTAGCCCTTC |
| 1148*- | (SEQ ID NO: 27) | TACAGTGGCCTTA |
| pegRNA | | (SEQ ID NO: 30) |
| PNPLA3- | AAGGATCAGGAAAATTAAAA | — |
| 1148- | (SEQ ID NO: 28) | |
| PE3- | | |

sgRNA

To provide Cas9 a plasmid expressing SpCas9 as well as mCherry for visualization of transfected cells (Addgene #66940) was used. To enrich for transfected cells, a two-plasmid transposon system comprised of a piggyBac transposase and a donor plasmid with terminal repeats bearing a cassette with Hygromycin B resistance was used. Organoids were transfected by electroporation as described previously (Hendriks et al., 2021). Briefly, organoids were made into single cells using Accutase, washed twice with AdvDMEM+++, and resuspended in 130 μl Opti-MEM containing the DNA mixture. Typically, 1-2 wells of a 12-well plate (>70% confluency) were used per electroporation and a maximum amount of 20 μg of DNA was used. Electroporations were performed in 2-mm Nepa electroporation cuvettes with a NEPA21 Electroporator using the settings described in (Hendriks et al., 2021). After a 20 min recovery in HEP medium with additional Y-27632 (final concentration=10 μM), single cells were recovered, washed with AdvDMEM+++, and plated in 100 μl BME suspension into 1 well of a 12-well plate (3 droplets/well). Cells were cultured in complete HEP medium with additional Y-27632 (final concentration=10 μM), until small organoids appeared, after which organoids were shifted to regular HEP medium. When organoids were co-transfected with the piggyBac-Hygromycin B resistance transposon system, drug selection was started when organoids of small size had formed, typically 7-12 days after electroporation. Hygromycin B Gold (50 μg/μl) was kept until selection was visually complete (i.e. clear distinguishment between alive and dead organoids, typically 7-14 days). Single surviving organoids were picked, made into small fragments/single cells by Accutase, plated into a single BME droplet well of a 24-well plate, and expanded into clonal lines. When no selection strategy was used, organoids were picked based on visual phenotypes (e.g. APOB^{-/-} and MTTP^{-/-} mutants) and grown into clonal lines as described above.

### CRISPR Gene Knock-In

PLIN2 reporter lines in the background of APOB^{-/-} or MTTP^{-/-} organoids were generated using CRISPR-HOT as described previously (Artegiani et al., 2020; Hendriks et al., 2021). Briefly, the transfection mixture consisted of a sgRNA targeting the C-terminus of PLIN2, a plasmid providing both Cas9 and a non-human sgRNA targeting the donor plasmid (Addgene #66940), and a donor plasmid encoding either tdTomato (Addgene #138567) or mNeon (a gift from V. Hornung). Upon transfection, the sgRNA targeting the donor plasmid linearizes the donor plasmid to facilitate NHEJ-mediated in-frame gene knock-in of the fluorescent tag into the PLIN2 C-terminus. Upon outgrowth of transfected organoids, fluorescent organoids became apparent. A bulk PLIN2-tagged fluorescence-pure culture was established by sorting single fluorescence-positive cells by FACS. Genotyping was performed to confirm precise gene knock-in.

### CRISPR Prime Editing

PNPLA3 I148M and I148* mutations were introduced in organoids by PE3 prime editing (Anzalone et al., 2019). The pegRNAs and PE3 sgRNA to introduce the mutations were designed using the online web-tool pegFinder (http://pegfinder.sidichenlab.org/) (Chow et al., 2021). The pegRNAs were generated as previously described (Anzalone et al., 2019) and the PE3 sgRNA was cloned as described above. Spacer and 3′ extensions for the pegRNAs and the PE3 sgRNA are given in Table 1. In sum, the transfection mixture consisted of the specific pegRNA plasmid, containing the sgRNA and desired edit, the common PE3 sgRNA plasmid to induce the second nick, the PE2 plasmid (Addgene #132775), as well as a hygromycin-piggyBac two plasmid system to facilitate selection. Outgrowing hygromycin-resistant clones were picked and genotyped, and clonal lines were established as described above. To correct the I148M mutation, a pegRNA specific to I148M was used, and organoids (already hygromycin-resistant) were instead co-transfected with a GFP-piggyBac two plasmid system to facilitate selection based on GFP fluorescence.

### Genotyping of Mutant Lines

As soon as sufficient material was present, DNA was extracted from the clonal organoid lines and PCR reactions were performed to amplify the genomic region encompassing the sgRNA/Cas9-targeted area. PCR products were Sanger sequenced to confirm the genotypes. Genotypes were deconvoluted using the ICE v2 CRISPR tool (Hsiau bioRxiv) and if needed PCR products were further subcloned to discriminate between alleles.

### Steatosis Induction in Wild Type Organoids

Wild type human hepatocyte organoids were made steatotic by providing a concentrated mixture of exogenous FFAs (oleic acid (18:1) and palmitic acid (16:0), ratio 1:1) in the culture medium. The FFAs were dissolved in ethanol and thereafter conjugated to bovine serum albumin. Prior to fat loading, organoids were plated into small BME droplets (15 μl/drop) to facilitate FFA penetration. Total concentrations of FFA were 640 μM for transcriptomic analyses to mimic the level of steatosis observed in APOB^{-/-} or MTTP^{-/-} organoids.

### Drug Screening

Compound stocks of all drugs were made in DMSO, except for recombinant hFGF19 and hFGF21 which were reconstituted in AdvDMEM+++. Drugs were dissolved in HEP medium with a maximum final concentration of 0.2% DMSO. The drug panel, their targets, and concentrations tested are described in Table 2.

TABLE 2
Details of anti-NAFLD drug screening

| | | | Concentrations | |
|---|---|---|---|---|
| Drug | Target | Activation/ | tested | |
| Bempedoic acid | ATP citrate lyase (ACL) | Inhibition | 5-10-25-50 | μM |
| Firsocostat (GS-0976) | Acetyl-CoA carboxylase (ACC) | Inhibition | 0.1-1-2.5-10 | μM |
| Cardarine (GW-501516) | Peroxisome proliferator-activated | Activation | 0.1-1-2.5-10 | μM |
| | receptor d (PPARd) | | | |
| TVB2640 (ASC-40) | Fatty acid synthase (FAS) | Inhibition | 0.1-1-2.5-10 | μM |
| Aramchol | Stearoyl-CoA desaturase 1 (SCD1) | Inhibition | 0.1-1-2.5-10 | μM |
| SRT2104 | Sirtuin 1 (SIRT1) | Activation | 0.1-1-2.5-10 | μM |
| Resmetirom | Thyroid hormone receptor β (THRβ) | Activation | 0.1-1-2.5-10 | μM |
| (MGL-3196) | | | | |
| Cilofexor (GS-9674) | Farsenoid X receptor (FXR) | Activation | 0.1-1-2.5-10 | μM |
| Saroglitazar | Peroxisome proliferator-activated | Activation | 5-10-25-50 | μM |
| | receptor a and g (PPARa/g) | | | |
| Fenofibrate | Peroxisome proliferator-activated | Activation | 5-10-25-50 | μM |
| | receptor a (PPARa) | | | |
| Elafibranor | Peroxisome proliferator-activated | Activation | 5-10-25-50 | μM |
| | receptor a and d (PPARa/d) | | | |
| Lanifibranor | Peroxisome proliferator-activated | Activation | 5-10-25-50 | μM |
| | receptor a, g, and d (PPARa/g/d) | | | |
| Pioglitazone | Peroxisome proliferator-activated | Activation | 5-10-25-50 | μM |
| | receptor g (PPARg) | | | |
| PF06424439 | Diacylglycerol O-acyltransferase | Inhibition | 0.1-1-2.5-10 | μM |
| | 2 (DGAT2) | | | |
| AZD3988 | Diacylglycerol O-acyltransferase | Inhibition | 0.1-1-2.5-10 | μM |
| | 1 (DGAT1) | | | |
| Recombinant hFGF19 | β-Klotho/FGFRs | Activation | 25-100 | ng/ml |
| Recombinant hFGF21 | β-Klotho/FGFRs | Activation | 25-100 | ng/ml |
| SCH772984 | Extracellular signal-regulated | Inhibition | 0.001-0.01-0.05- | |
| | kinases | | 0.1 | |
| SB202190 | p38 mitogen-activated protein | Inhibition | 0.1-1-2.5-10 | μM |
| | kinases a and β (p38a/β) | | | |
| SP600125 | c-Jun N-terminal kinases 1, 2, | Inhibition | 0.1-1-2.5-10 | μM |
| | and 3 (JNK1/2/3) | | | |

APOB^{-/-} and MTTP^{-/-} organoids were exposed to the drugs or vehicle in 24-well plates for 7 days with 2 medium changes. Wild type organoids were first made steatotic by pre-incubation with 500 μM FFAs (oleic acid and palmitic acid, 1:1 ratio) for 2 days. Then, FFA-loaded organoids were treated with the different drugs, still in the presence of FFA, for 7 days with 2 medium changes. All organoids were harvested for subsequent lipid staining and lipid scoring as described below. Drug effects were evaluated by observing the lipid droplet fluorescence characteristics within all organoids within the whole well. Quantitative analyses were performed in representative organoids (n≥3) per drug concentration per steatosis organoid model. Drug effects were validated in at least two independent experiments. For drug screening in PLIN2::tdTomato reporter organoids, the organoids were plated in 96-well black plates and treated with the indicated drugs. Pictures were taken over a 7-day time course using the EVOS FL Auto Imaging System. Images and fluorescence quantifications are representative of two independent experiments.

### CRISPR Screening

APOB^{-/-} or MTTP^{-/-} organoid lines generated without prior hygromycin resistance selection were used for CRISPR screening. Organoids were transfected with the sgRNA plasmid targeting the gene of interest, a Cas9-expressing plasmid, and the two-plasmid hygromycin-based transposon system as described above. The genes evaluated are given in Table 3 and their sgRNA sequences are given in Table 1.

TABLE 3
Details of genes evaluated with CRISPR screening

| | Log2FC in APOB^{-/-} vs. wild | p value in APOB^{-/-} vs. wild |
|---|---|---|
| Gene | type organoids | type organoids |
| ACACA, ACACB* | ACACA: −0.521522058 | ACACA: 0.002773846 |
| | ACACB: −0.270357217 | ACACB: 0.149101512 |
| ACE2 | −2.278515982 | 1.34E−06 |
| ACSL1 | −1.092347479 | 8.91E−05 |
| AKR1C1 | −0.92315025 | 0.000387205 |
| AKR1C2 | −0.819020912 | 0.002027809 |
| BHLHE40 | −1.097343182 | 0.000348402 |
| DGAT2 | −0.599894276 | 5.25E−05 |
| FADS2 | −1.105466721 | 5.53E−09 |
| FASN | −0.935657328 | 3.64E−07 |
| LPCAT1 | −0.924830459 | 0.004561172 |
| LSS | −1.042010382 | 2.66E−09 |
| MOGAT3 | −1.099144079 | 4.69E−07 |
| MVD | −0.966445261 | 4.16E−06 |
| PCSK9 | −0.837433763 | 0.000193329 |
| PLIN1 | −1.236192238 | 0.001097479 |
| SCARB1 | −0.710986629 | 0.001634826 |
| SERPINE1 | −1.804506692 | 0.002245518 |
| SLC29A3 | −1.142474986 | 0.001021598 |
| STARD8 | −0.90246434 | 0.003538497 |
| TRIB3 | −1.466272866 | 2.96E−05 |

*Considered not because of significance but to ensure complete loss of ACC

Outgrowing hygromycin-resistant organoids were carefully inspected by light microscopy and compared to control surviving organoids (mock transfected organoids with Cas9 and a non-human sgRNA). Upon indications of different phenotypes between surviving organoids (i.e. impacting on the steatosis phenotype), organoids were picked, grown into clonal lines and genotyped. Alternatively, organoids were directly processed for lipid staining when clonal line establishment was not possible. CRISPR experiments were repeated at least two times independently using both APOB^{-/-} and MTTP^{-/-} lines. Significant findings were followed up in multiple (n≥3) clonal lines from independent experiments.

### Transmission Electron Microscopy

Organoids were fixed with 1.5% glutaraldehyde in 0.1 M cacodylate buffer at 4° C. for 24 hours. Then, organoids were washed with 0.1 M cacodylate buffer and postfixed with 1% osmium tetroxide in the same buffer containing 1.5% potassium ferricyanide in the dark at 4° C. for 1 hour. The samples were dehydrated in ethanol, infiltrated with Epon resin for 2 days, embedded in the same resin and polymerized at 60° C. for 48 hours. Ultrathin sections were cut using a Leica Ultracut UCT ultramicrotome (Leica Microsystems Vienna) and mounted on Formvar-coated copper grids. The sections were stained with 2% uranyl acetate in 50% ethanol and lead citrate. Sections were observed in a Tecnai T12 Electron Microscope equipped with an Eagle 4k×4k CCD camera (Thermo Fisher Scientific, The Netherlands). To specifically preserve and visualize lipid droplet morphology, organoids (fixed similarly as described above) were instead high-pressure frozen using a Leica HPF. Freeze substitution was performed in a Leica AFS2 using 2% osmium tetroxide, 0.1% glutaraldehyde and 5% water in acetone. The temperature was raised from −90° C. to 20° C. with a rate of 5° C. per hour. After three washes with acetone, the samples were infiltrated and embedded in Epon and polymerized as described above. Ultrathin sections were observed in a Tecnai T12 Electron Microscope as mentioned.

### Lipid and Immunofluorescence Staining

Organoids were carefully harvested in cold AdvDMEM+++ and washed once with cold AdvDMEM+++ using low-speed centrifugations. Organoids were fixed in 4% formaldehyde at RT for 30 min-1 hour. For lipid stainings, organoids were washed twice with PBS, and incubated with Nile Red (1 μg/μl) and DAPI (1 μg/μl) for 20 min at RT. Organoids were washed twice with PBS, and transferred in 100 μl PBS to a well of a 96-well black SensoPlate for imaging analysis. For immunofluorescence stainings, fixed organoids were first washed twice with PBS, and then simultaneously blocked and permeabilized using 5% BSA and 0.3% Triton-X in PBS at RT for 1 hour. Organoids were washed once with 0.5% BSA-PBS and subsequently incubated with primary antibodies in 2.5% BSA-PBS O/N at 4° C. After 3 washes with 0.5% BSA-PBS, organoids were incubated with secondary antibodies in 2.5% BSA-PBS for 2-4 hours at RT. Organoids were washed once with 0.5% BSA-PBS, after which they were incubated with DAPI (1 μg/μl) in 0.5% BSA-PBS for 20 min at RT, and washed once more with 0.5% BSA-PBS. Organoids were then transferred in 100 μl 0.5% BSA-PBS to a well of a 96-well black SensoPlate.

### Confocal Imaging and Lipid Scoring

Stained organoids were imaged on a Leica Sp8 confocal. Fluorescent images were processed using Photoshop CS4 or ImageJ software. The lipid score was calculated using Fiji software. The lipid score is defined by integrating the lipid droplet fluorescence and lipid droplet area coverage as follows: a fluorescence threshold derived from the lipid droplet signal is determined on Z-projected images to convert them into binary images. The region of interest (ROI) (organoid surface area) is defined based on fluorescence signal from the counterstained DAPI⁺ nuclei. Then, particle measurement analysis is performed to define the fluorescence area covered within the defined ROI. The lipid score represents a normalized score of the resulting data on a linear 0 to 1 scale, where the mean calculated values from wild type organoids are arbitrarily set to 0, while the mean calculated values from either vehicle-treated APOB^{-/-} or MTTP^{-/-} organoids or vehicle-treated FFA-loaded organoids are set to 1, allowing scoring of drug effectiveness within these boundaries. Quantification of the amount of lipid droplets was performed manually by counting droplets within a given ROI (organoid surface area). The lipid droplet area coverage is determined identical to the lipid score without the final normalization steps.

### Bulk RNA-Sequencing

Organoids from 1 well of a 12-well plate were harvested and washed in cold AdvDMEM+++. Organoid pellets were lysed in 1 ml TRIzol Reagent and subsequently snap-frozen in liquid nitrogen. RNA was extracted according to the manufacturer's protocol. RNA integrity was measured using the Agilent RNA 6000 Nano kit with the Agilent 2100 Bioanalyzer and RNA concentrations were determined using the Qubit RNA HS Assay Kit. RNA integrity number (RIN) values of RNA samples were typically 9.5-10 and never below 9.0. RNA libraries were prepared TruSeq Stranded mRNA polyA kit and single-end (1×75 bp) sequenced on an Illumina Nextseq 2000 or Nextseq 500. Reads were mapped to the human GRCh38 genome assembly. Differential gene expression analysis was performed using the DESeq2 package (Love et al., 2014) in RStudio. Considered log 2 fold changes and significance (p-values) are indicated throughout the paper. Data visualization was obtained using the ggplot2, ComplexHeatmap, and EnhancedVolcano packages in RStudio or manually plotted using GraphPad Prism 8.

### Lipidomics

Organoids (all samples plated at the same density) were cultured for 4 days without changing the medium. Then, the medium (supernatant) from 1 well of a 12-well plate was collected, spun down to remove cell debris, and snap-frozen in liquid nitrogen. Organoids from the same well were harvested in cold AdvDMEM+++, washed twice with cold AdvDMEM+++, and dry organoid pellets were snap-frozen in liquid nitrogen. Lipids from cell pellets and medium (0.5 ml) were extracted with chloroform and methanol as described previously (Bligh and Dyer, 1959). Lipid extracts were kept frozen under a nitrogen atmosphere until analysis. Neutral lipid analysis was performed on a ACQUITY Premier BEH C18 column (130 Å, 1.7 μm 2.1×100 mm) from Waters (Milford, MA). Elution was performed at 60° C. using a binary gradient from (A) methanol:water (50:50 v/v) to (B) methanol:isopropanol:ethyl acetate (80:12:8 v/v/v). Both solvents contained 10 mM ammonium formate. Gradient composition was (time, % B):(0, 60); (2.5, 100); (8, 100); (8.1, 60); (10, 60) and the flow rate was kept constant at 0.4 ml/min. The column effluent was introduced into a X500R QToF type mass spectrometer (Sciex, Framingham, MA), either via an atmospheric pressure chemical ionization (APCI) source or a heated electrospray ionization (HESI) source both operated in the positive ion mode. Data from the APCI interfaced runs were used to determine oxysterols, sterols, sterol esters and total TAG content. Data from the subsequent HESI run were used to determine molecular species composition of TAG as ammonium adducts. Data analysis was performed using the XCMS package in R, essentially as described elsewhere (Jeucken et al., 2019; Smith et al., 2006). Principal component analysis was performed using the non-linear iterative partial least squares (nipals) method using pareto scaling (Stacklies et al., 2007).

### FADS2 Overexpression

To overexpress human FADS2, we generated a transposable construct for use in conjunction with the piggyBac transposase. FADS2 cDNA was generated from RNA from human hepatocyte organoids using the SuperScript IV kit. Using PBCAG-eGFP (Addgene #40973) as a backbone, we replaced the eGFP sequence with FADS2 using In-Fusion cloning to generate CAG-FADS2 plasmid. The FADS2-P2A-tdTomato plasmid was generated in a similar manner using P2A-tdTomato as additional cloning insert. FADS2 overexpression was confirmed by RT-qPCR analysis using iQSYBRGreen mix with the qPCR primers FADS2_fw: 5′ GACCACGGCAAGAACTCAAAG 3′ (SEQ ID NO: 35) and FADS2_rev: 5′ GAGGGTAGGAATCCAGCCATT 3′ (SEQ ID NO: 36).

### Lipid Chain Length Analysis

Lipids from cell pellets and medium (0.5 ml) were extracted with chloroform and methanol. Lipid extracts were kept frozen under a nitrogen atmosphere until analysis. Neutral lipid analysis was performed on a ACQUITY Premier BEH C18 column (130 Å, 1.7 μm 2.1×100 mm) from Waters (Milford, MA). Elution was performed at 60° C. using a binary gradient from (A) methanol:water (50:50 v/v) to (B) methanol:isopropanol:ethyl acetate (80:12:8 v/v/v). Both solvents contained 10 mM ammonium formate. Gradient composition was (time, % B):(0, 60); (2.5, 100); (8, 100); (8.1, 60); (10, 60) and the flow rate was kept constant at 0.4 ml/min. The column effluent was introduced into a X500R QToF type mass spectrometer (Sciex, Framingham, MA), either via an atmospheric pressure chemical ionization (APCI) source or a heated electrospray ionization (HESI) source both operated in the positive ion mode. Data from the APCI interfaced runs were used to determine oxysterols, sterols, sterol esters and total TAG content. Data from the subsequent HESI run were used to determine molecular species composition of TAG as ammonium adducts.

### De Novo Lipogenesis Index Analysis

The de novo lipogenesis (DNL) index calculated based on the C16:0/C18:2 ratio. C16:0, palmitic acid, represents the main lipogenesis product and C18:2, linolenic acid, represents the diet-derived essential fatty acid.

### Example 1

### Results

### Development of Genetically-Engineered Steatosis Organoids

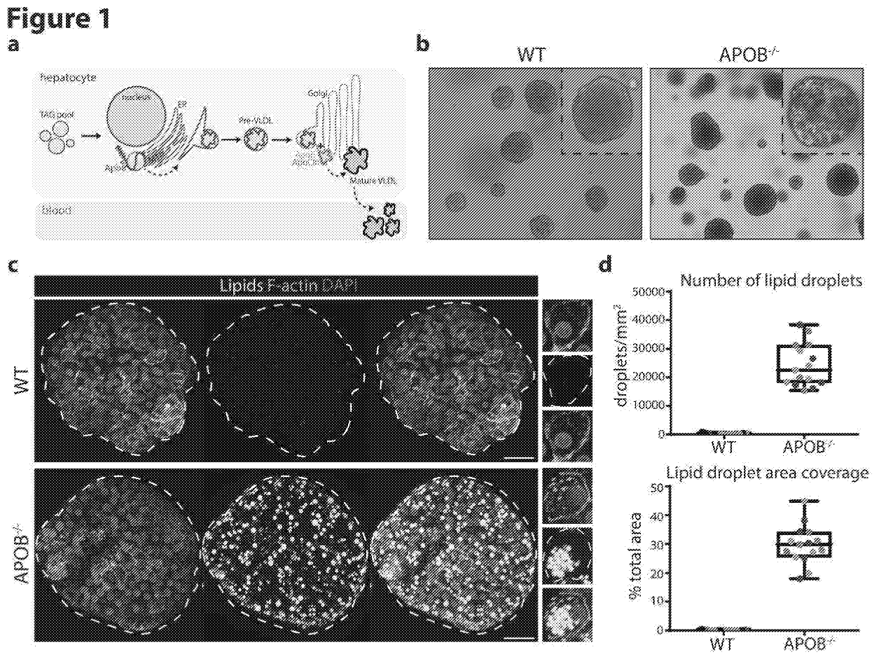

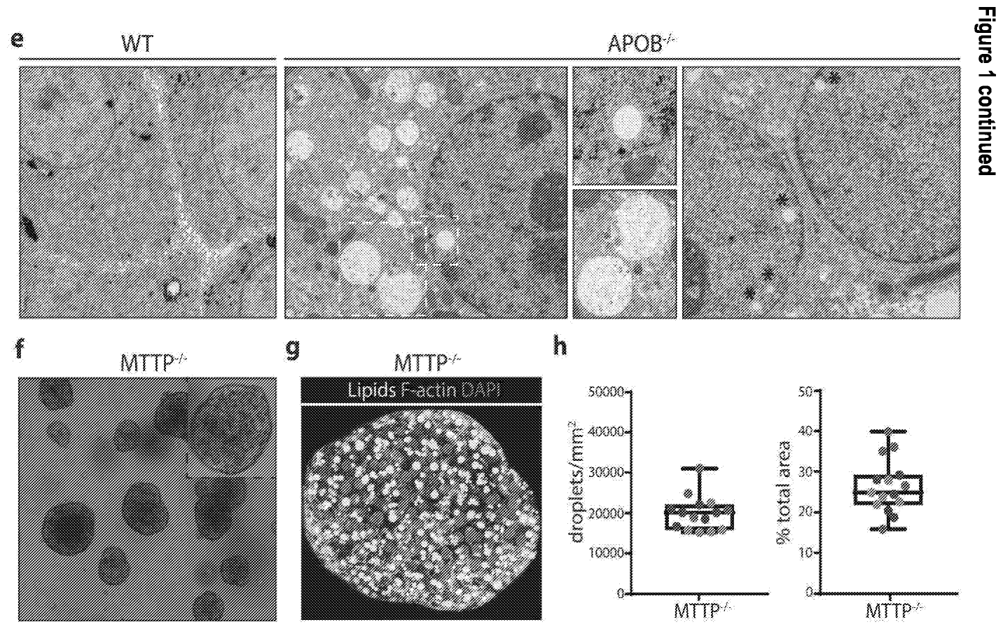

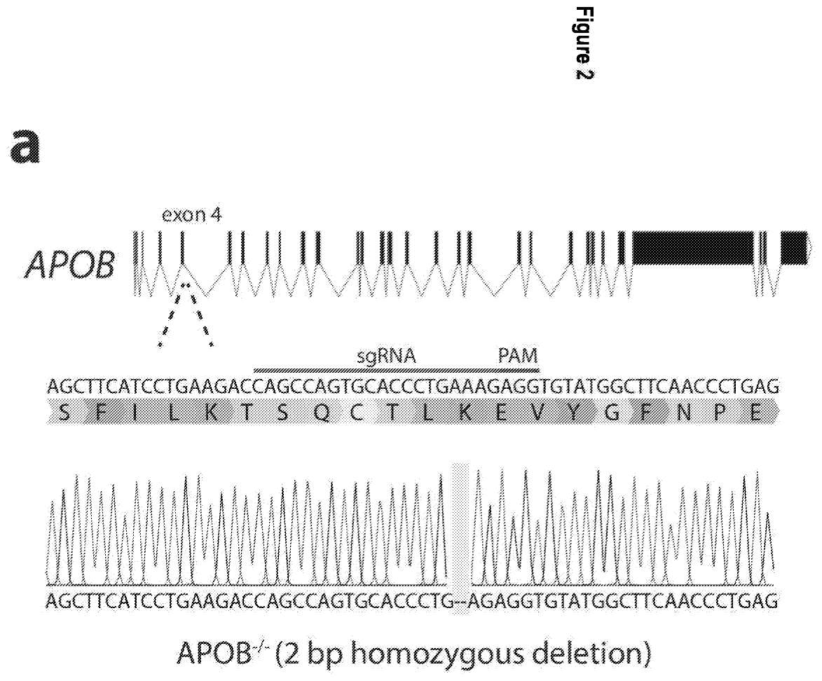

The APOB gene encodes apolipoprotein B (ApoB), an essential structural component of very-low-density lipoproteins (VLDL). Lipids, either derived from circulating free fatty acids or generated de novo from carbohydrates, are secreted by hepatocytes into the serum as constituents of these VLDL particles (FIG. 1a). APOB gene mutations cause familial hypobetalipoproteinemia (FHBL) (Schonfeld et al., 2003a): impaired VLDL secretion causes decreased triglyceride export from the liver and thus may induce steatosis. Consequently, APOB-mutant individuals are at risk to develop NAFLD, NASH, and liver cancer (Cefalù et al., 2013; Schonfeld et al., 2003b). The possibility to study NAFLD from a genetic angle through CRISPR-mutating the APOB locus in wild type human hepatocyte organoids was explored (Hendriks et al., 2021; Hu et al., 2018).

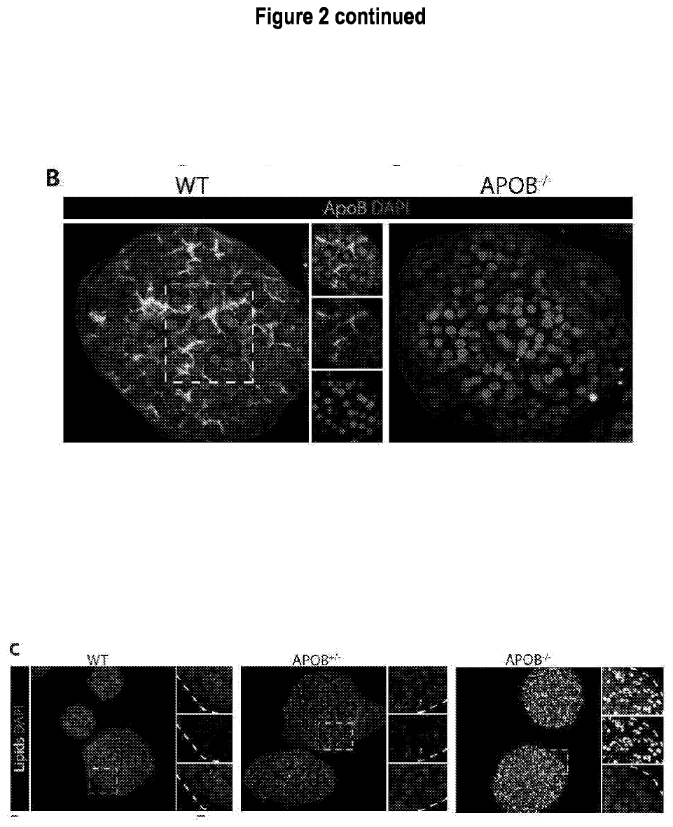

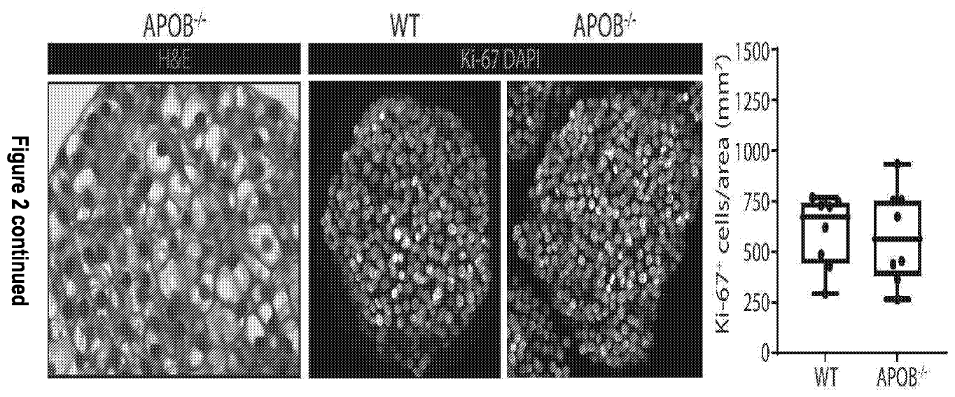

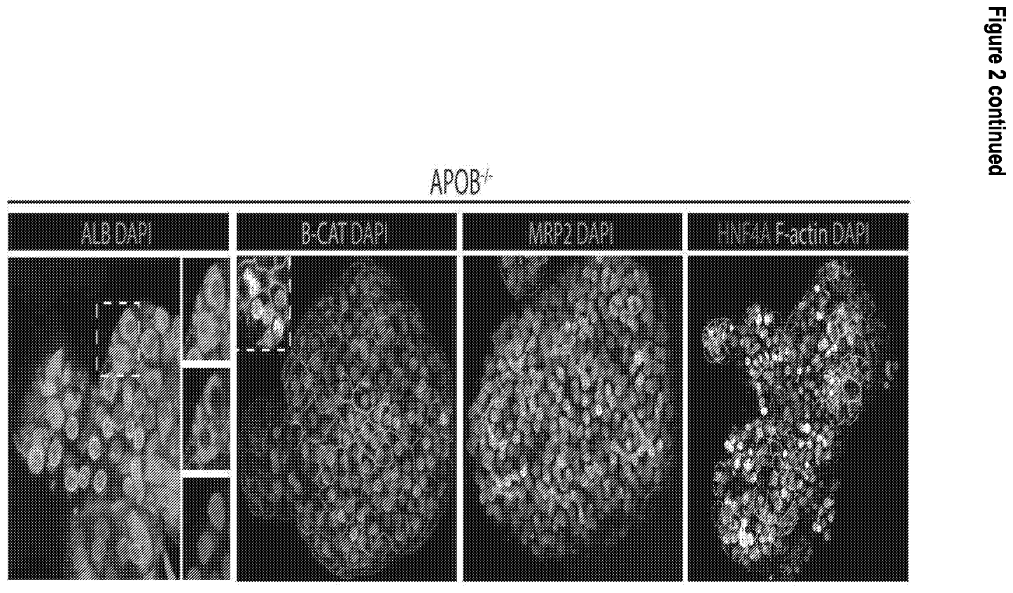

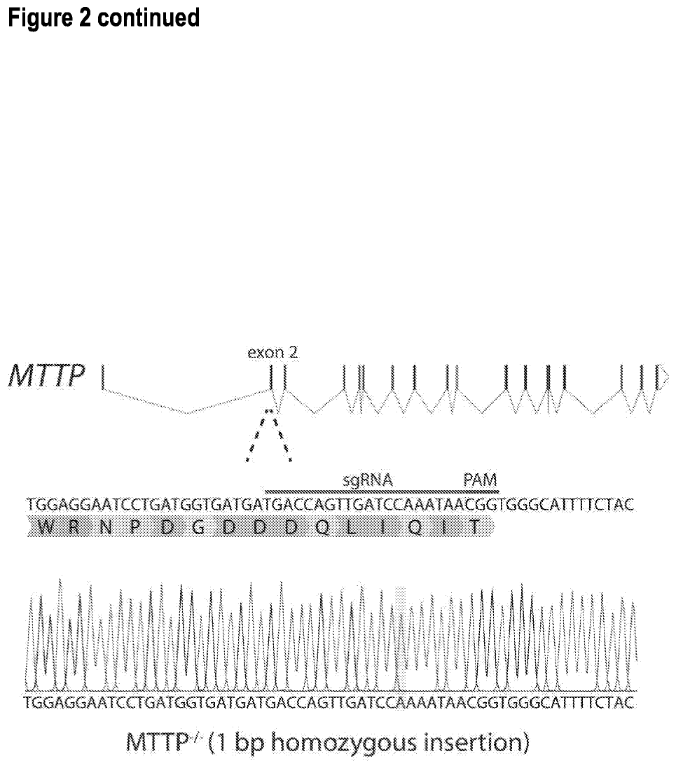

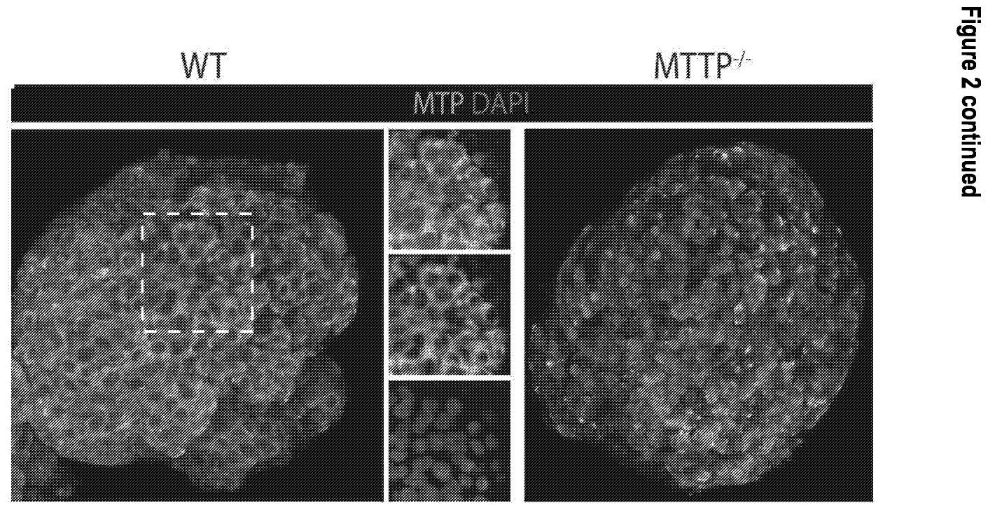

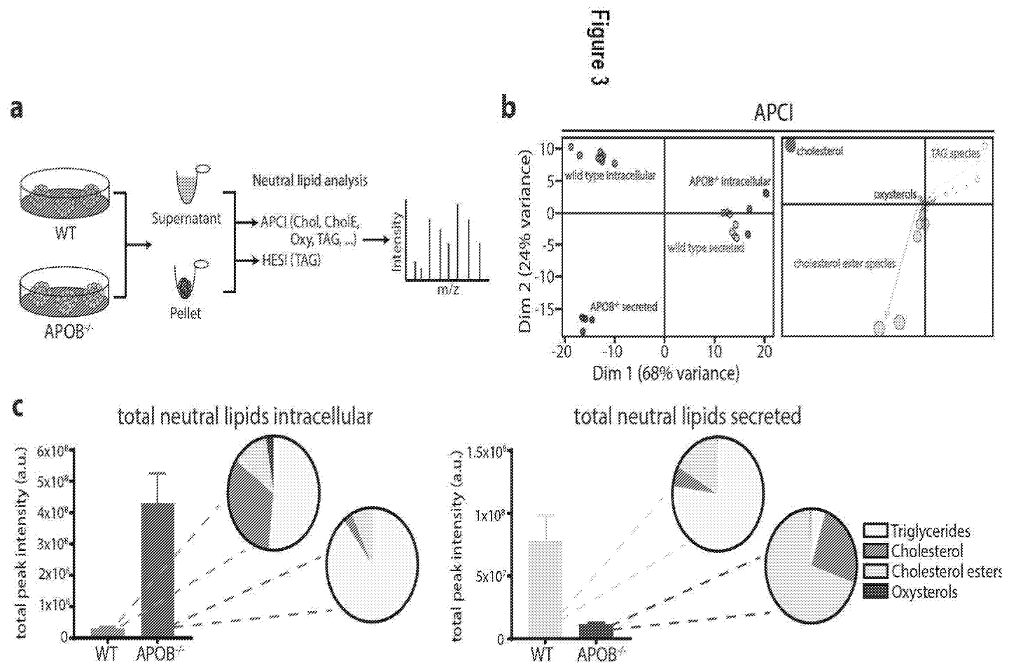

Clonal organoid lines homozygously mutant for APOB from multiple donors could be derived. Frameshift mutations and consequent loss of ApoB was confirmed by immunofluorescence (FIG. 2a-b). APOB^{-/-} mutants were visually darker with an abundance of lipid droplets (FIG. 1b), while heterozygous mutants accumulated only few lipid droplets (FIG. 2c). Nile Red and H&E stainings confirmed the accumulation of intracellular lipid droplets throughout the APOB^{-/-} organoids (FIG. 1c-d, FIG. 2d), whereas wild type organoids do not spontaneously accumulate lipid droplets under normal culture conditions (FIG. 1c-d). The lipid droplets occupied ca. 30% of the area of APOB^{-/-} organoids (FIG. 1d), equivalent to grade 1-2 steatosis of the human liver (Kleiner et al., 2005). The presence of lipid droplets of varying size throughout the cell was readily visualized by transmission electron microscopy (FIG. 1e). The majority of droplets resided in the cytoplasm, but lipid droplets accumulating in the nucleus was also evidenced. Phenotypic characterization of these organoids revealed that proliferation rates and expression of hepatocyte and structural markers were unaffected by the lipid accumulation, when compared to wild type organoids (FIG. 2e-f).

Next the phenotypic effects upon knock-out of MTTP, which encodes microsomal triglyceride transfer protein (MTP), essential for lipid transfer to ApoB for assembly of lipoprotein particles (FIG. 1a) was evaluated. Mutations in MTTP are causative of abetalipoproteinemia (ABL) (Wetterau et al., 1992), a disease that shares most of its clinical features with FHBL, including susceptibility to develop liver steatosis (Welty, 2014). Indeed, MTTP^{-/-} organoids (FIG. 2g-h) phenocopied APOB^{-/-} organoids (FIG. 1f), developing spontaneous steatosis under normal culture conditions (FIG. 1g) and bearing a very similar lipid accumulation profile (FIG. 1h). Since there are minimal fat sources present in the culture medium, APOB^{-/-} and MTTP^{-/-} organoids develop steatosis through accumulation of lipids derived from de novo lipogenesis using carbohydrates from the culture medium. Collectively, APOB- and MTTP-mutant organoids provide a novel genetic platform to study de novo lipogenesis-driven steatosis.

### Lipid Profiles and Transcriptomic Make-Up of Genetically-Engineered Steatosis Organoids

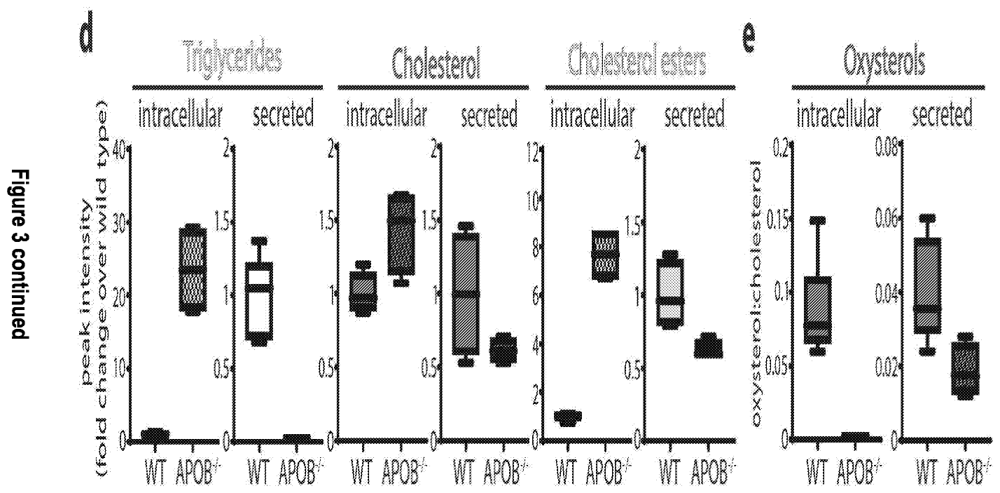

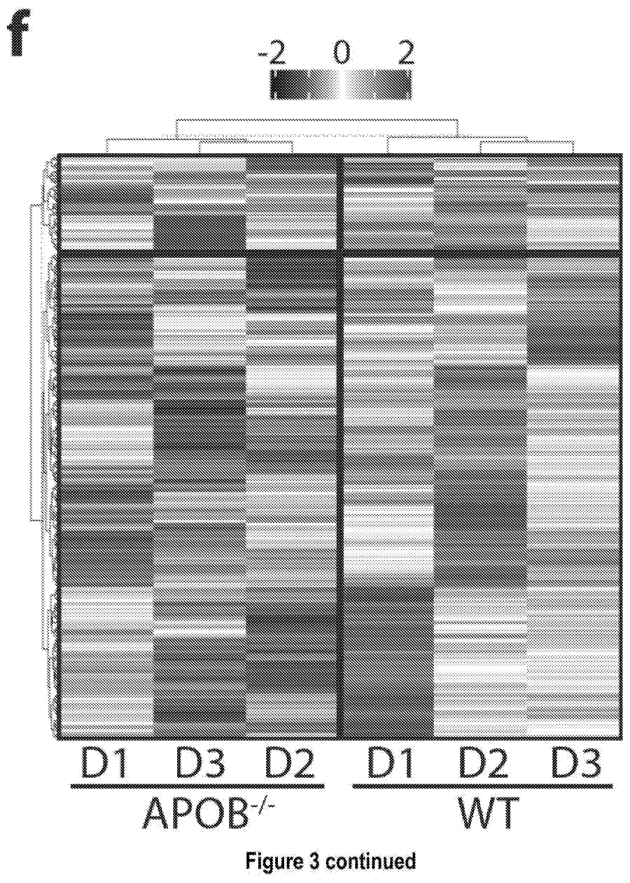

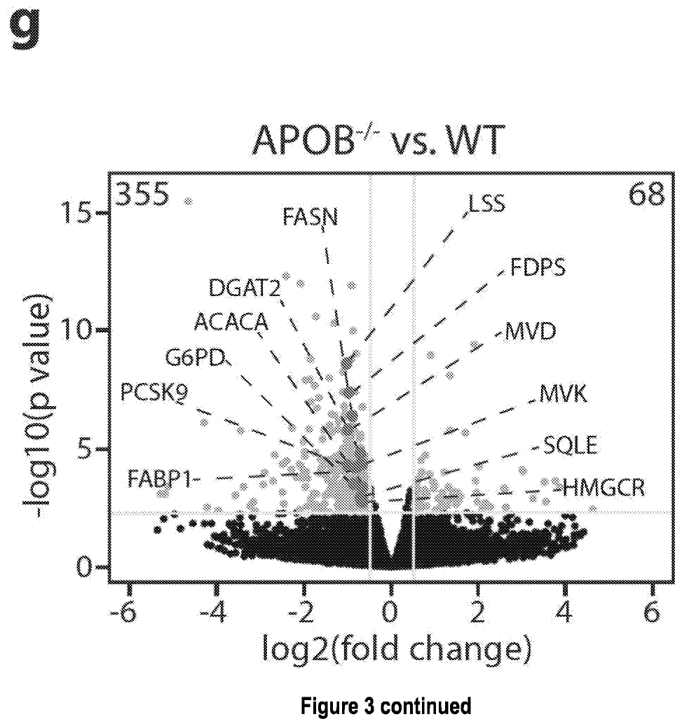

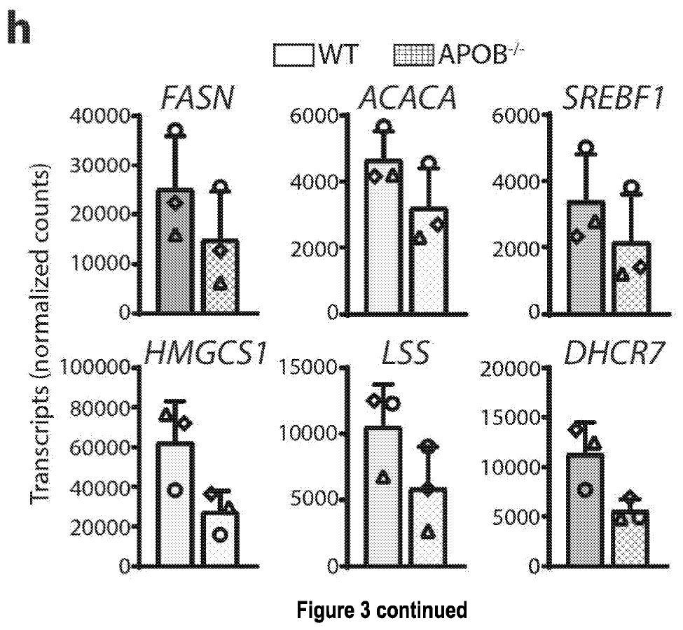

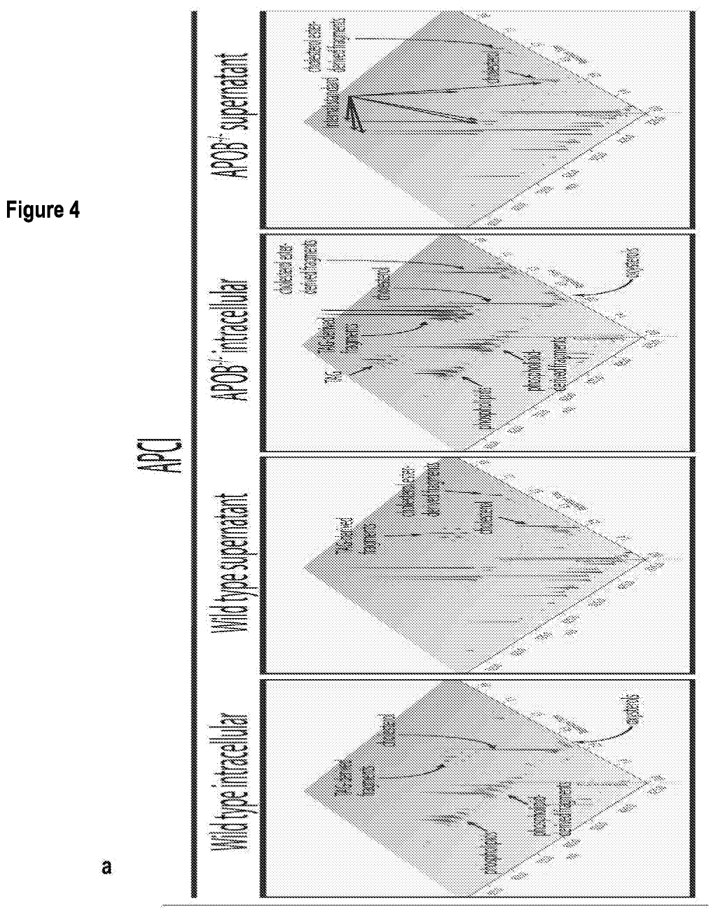

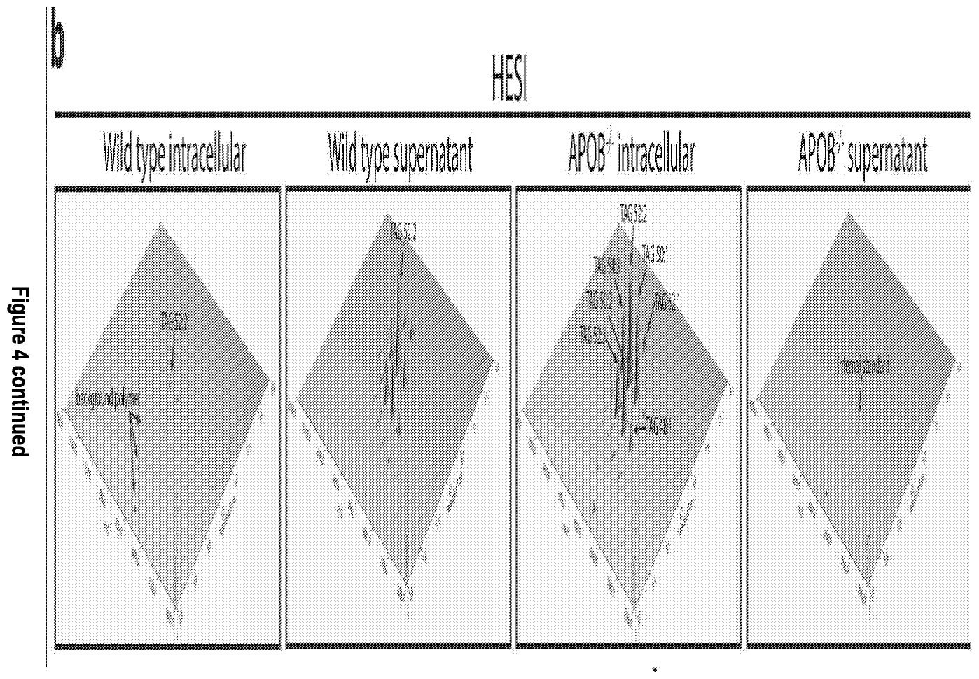

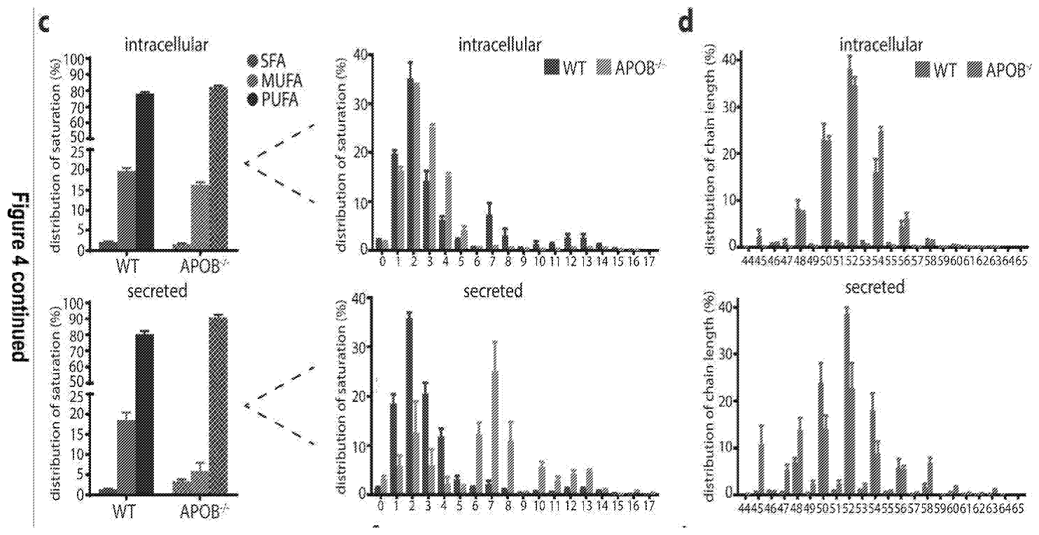

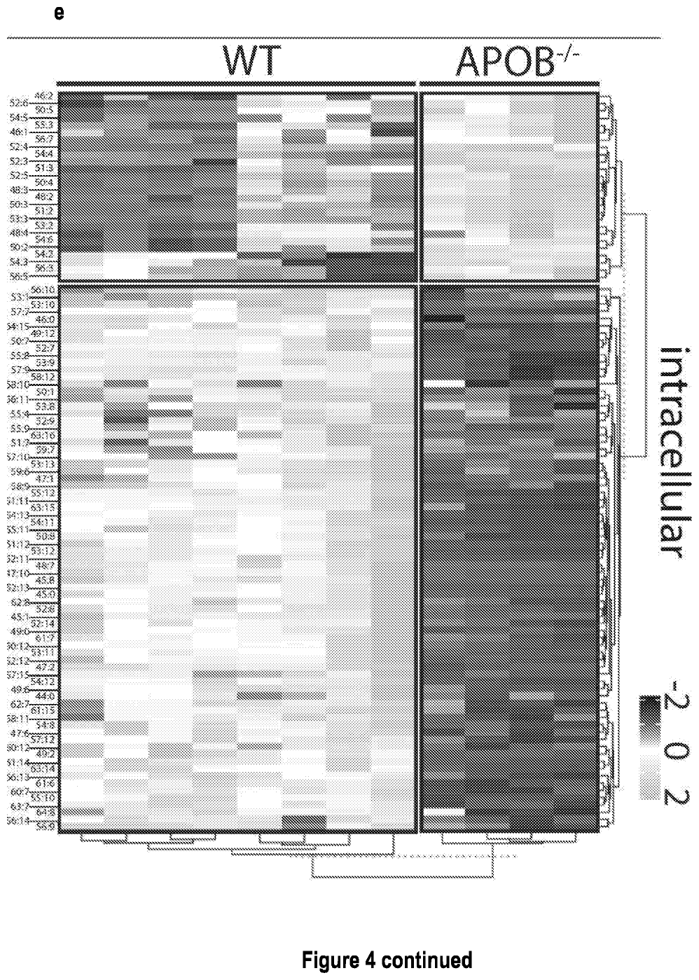

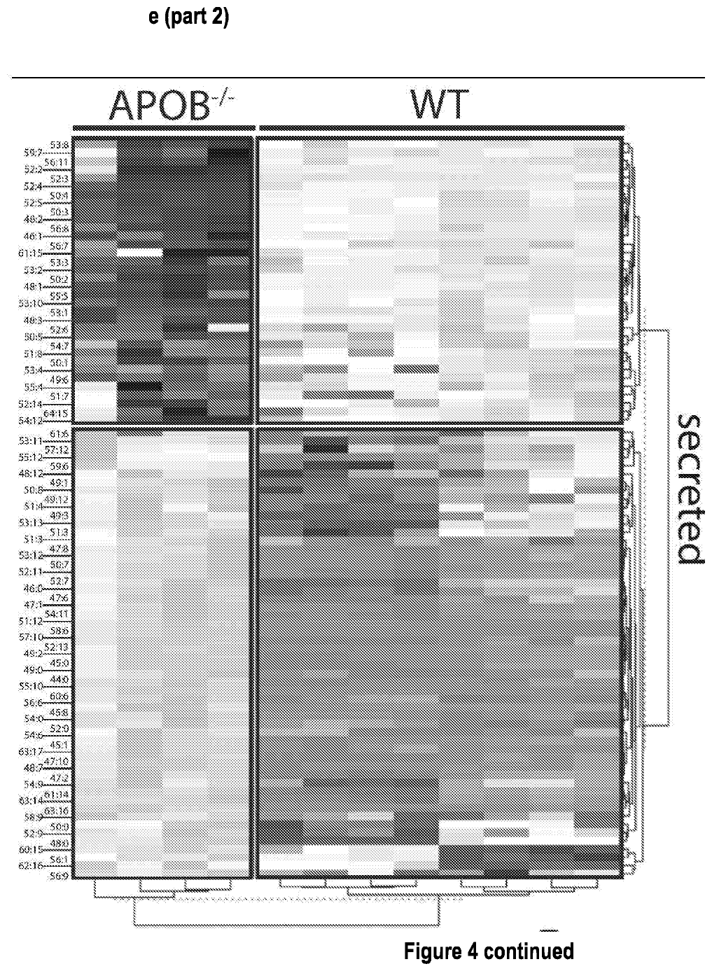

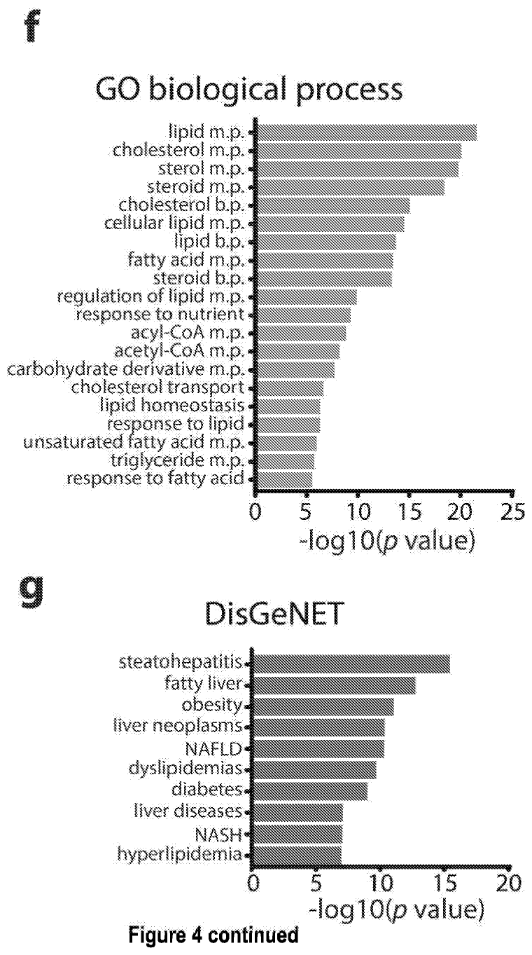

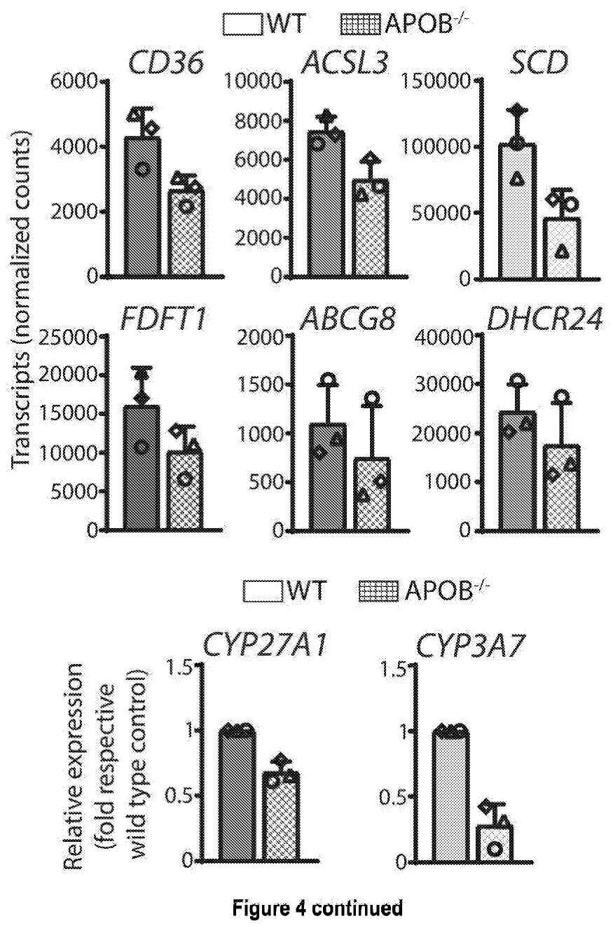

Next the intracellular (pellet) and secreted (supernatant) lipid profiles of APOB^{-/-} organoids was interrogated in comparison to wild type organoids using a lipidomic approach (FIG. 3a). Atmospheric-pressure chemical ioniziation (APCI) captured all neutral lipid classes (e.g. triacylglycerols (TAG), cholesterol (Chol), cholesterol esters (CholE), oxysterols (Oxy)), while with heated electrospray ionization (HESI) it was possible to deconvolute TAG complexity (FIG. 4a-b). Principal component analysis revealed three clusters: 1) the intracellular lipid profiles of wild type organoids, 2) the secreted lipid profiles of APOB^{-/-} organoids, and 3) a cluster comprised of both the intracellular profiles of APOB^{-/-} organoids and the secreted lipid profiles of wild type organoids, indicating that loss of ApoB leads to substantial changes in the intracellular lipidome and impairment of lipid secretion (FIG. 3b). In line, it was found that total neutral lipids were highly elevated within APOB^{-/-} organoids, while lipid secretion was starkly reduced (FIG. 3c).

Major differences in lipid composition were also noted. Lipids within APOB^{-/-} organoids were largely dominated by TAG species, while the few lipids present in wild type organoids were instead more distributed between TAG, Chol and CholE. Lipids secreted by wild type organoids were mainly composed of TAGs with smaller amounts of CholE and Chol. Instead, the few lipids secreted by APOB^{-/-} organoids were predominantly composed of CholE and Chol (FIG. 3c). In absolute terms, APOB^{-/-} organoids accumulated TAGs intracellularly at ca. 25-fold higher levels relative to wild type organoids, while TAG secretion was near absent (FIG. 3d). TAG compositions were also majorly altered (FIG. 4c-e): the few TAGs APOB^{-/-} organoids could secrete were preferentially saturated or poly-unsaturated fatty acids (PUFAs) but not mono-unsaturated fatty acids (MUFAs), while in wild type organoids valuable high PUFAs were retained intracellularly and secretion of MUFAs was more pronounced. CholE species also accumulated intracellularly in APOB^{-/-} organoids (ca. 8-fold), while secretion was reduced but not absent (FIG. 3d). Free Chol differs from CholE and TAG by being a structural membrane component. Indeed, free Chol amounts were much less drastically impacted, but with a trend similar to TAG and CholE (FIG. 3d). Notably, despite an overall accumulation of lipids intracellularly in the APOB^{-/-} organoids, oxysterols were near absent, while being clearly present intracellularly in wild type organoids (FIG. 3e).

To gain insights into the cellular responses towards the steatosis phenotype of APOB^{-/-} organoids, bulk RNA-sequencing was performed. Differential gene expression analysis revealed similar transcriptomic responses of APOB^{-/-} organoids established from 3 different donors (FIG. 3f). 423 genes were found to be significantly differently expressed when compared to donor-matched wild type organoids (FIG. 3g). GO-term analysis on the differentially expressed genes (DEGs) revealed many hits related to cholesterol and lipid metabolism (FIG. 4f), while DisGeNET analysis on DEGs revealed a high enrichment for fatty liver and related diseases (FIG. 4g). Specifically, APOB^{-/-} organoids were characterized by a collective downregulation of key genes involved in hepatic de novo lipogenesis (FIG. 3g-h). These included amongst others ACACA, FASN, DGAT2, as well as the master transcription factor SREBP-1c (SREBF1). A similar trend for genes involved in cholesterol biosynthesis (e.g. HMGCS1, SQLE, LSS, DHCR7) was also noted. Closer evaluation of the DEGs revealed that many of these genes are regulated by liver X receptors (LXRs) (e.g. FASN, SREBF1, etc.) (FIG. 3h, FIG. 4i), providing a possible link with the observed near absence of intracellular oxysterols which are known activators of LXR (Calkin and Tontonoz, 2012). Oxysterols are generated from cholesterol, amongst others, by different cytochrome P450 (CYP) enzymes in the liver (Luu et al., 2016). In line, significant downregulation of several relevant CYPs, in particular CYP27A1 and CYP3A7/4 (FIG. 4j) were noted.

Transcriptomic Responses of Steatosis Organoids Resulting from Genetics Versus Dietary Challenge

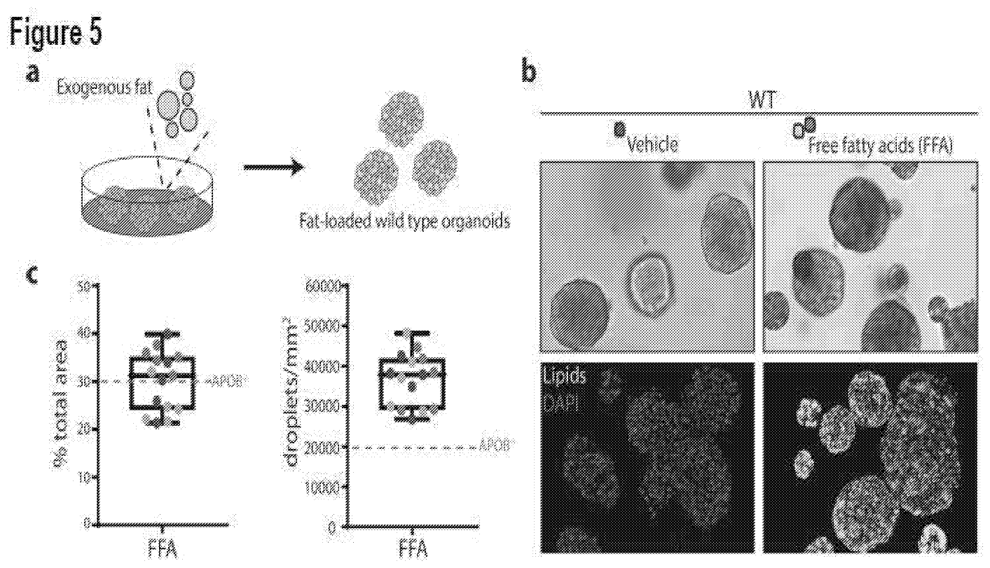

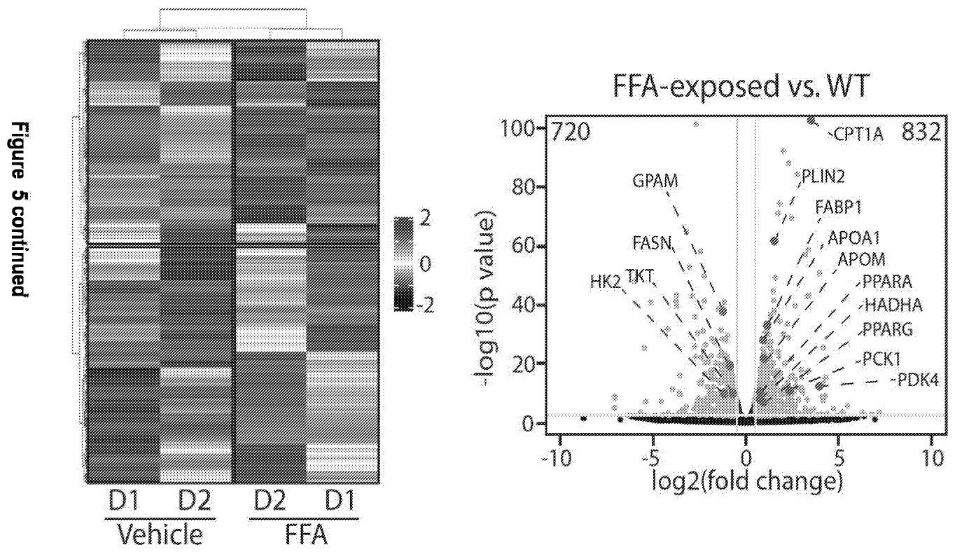

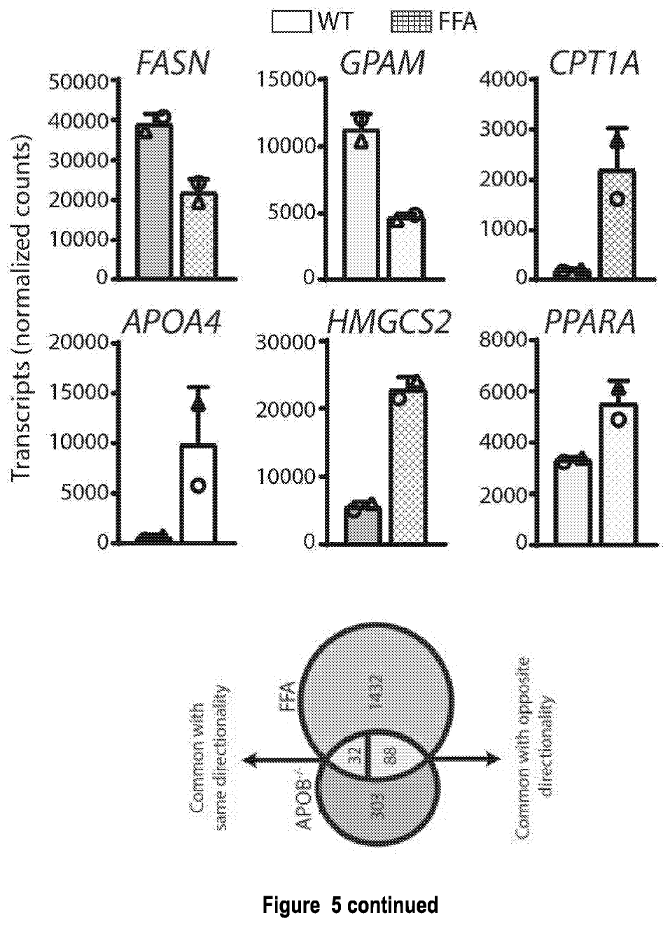

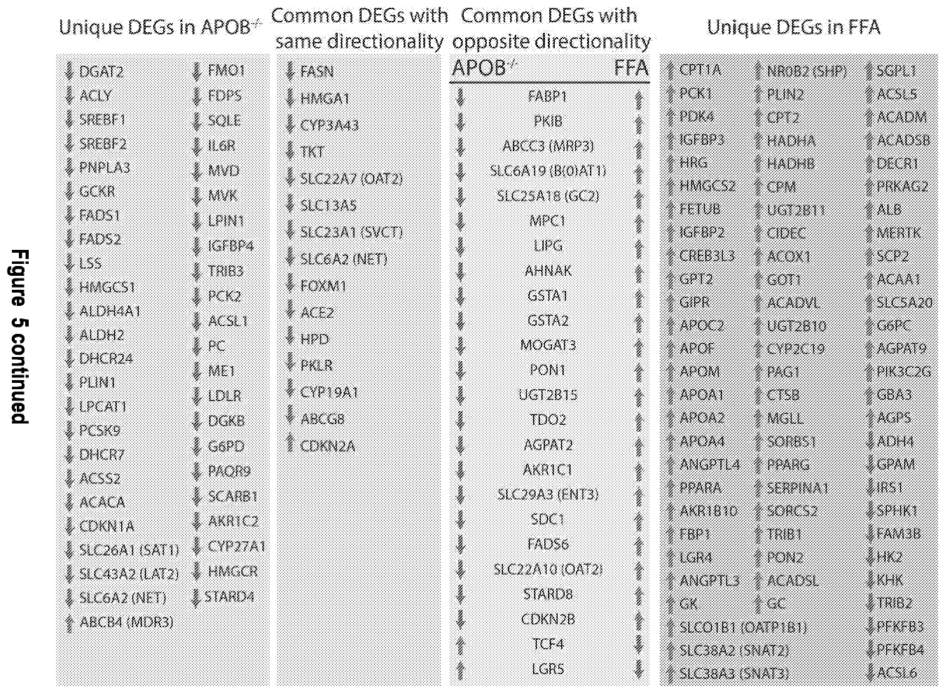

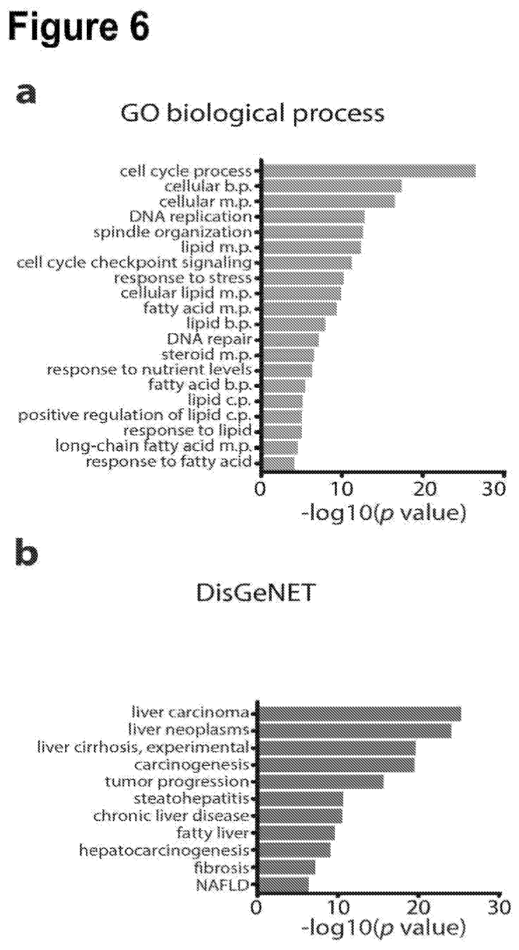

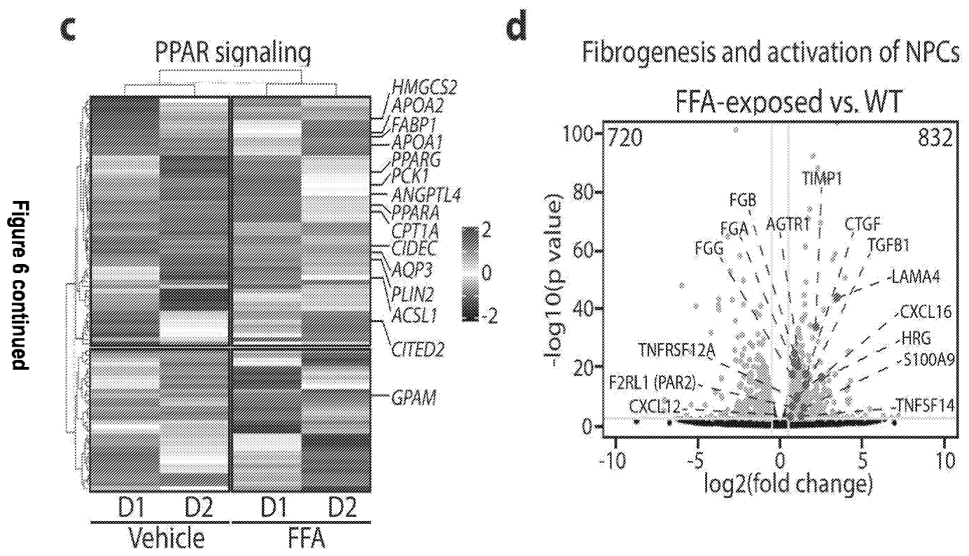

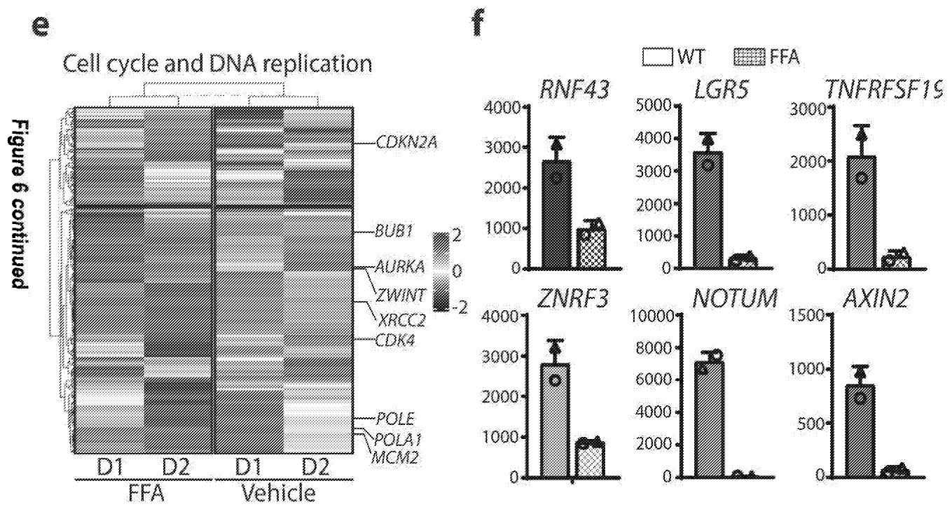

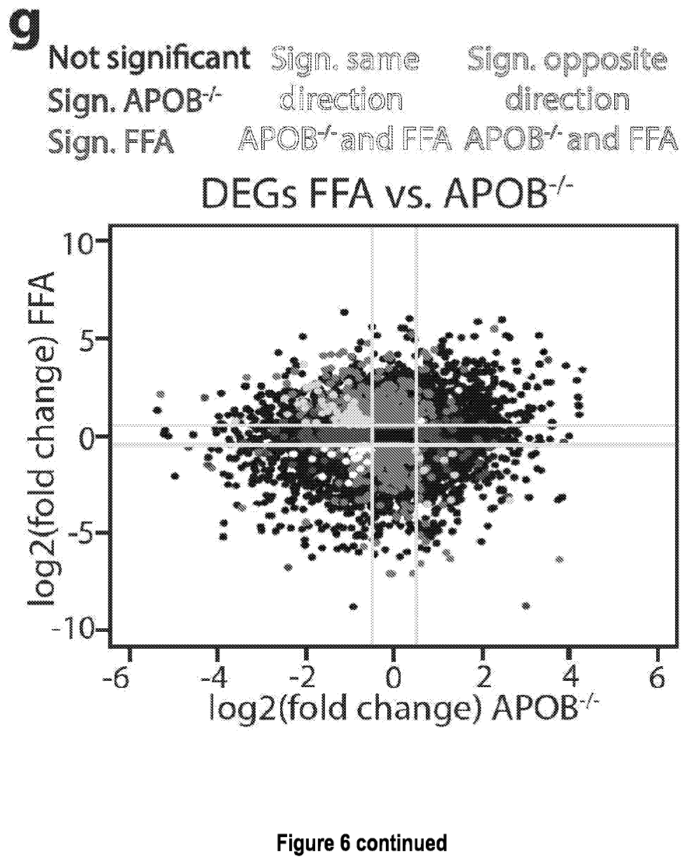

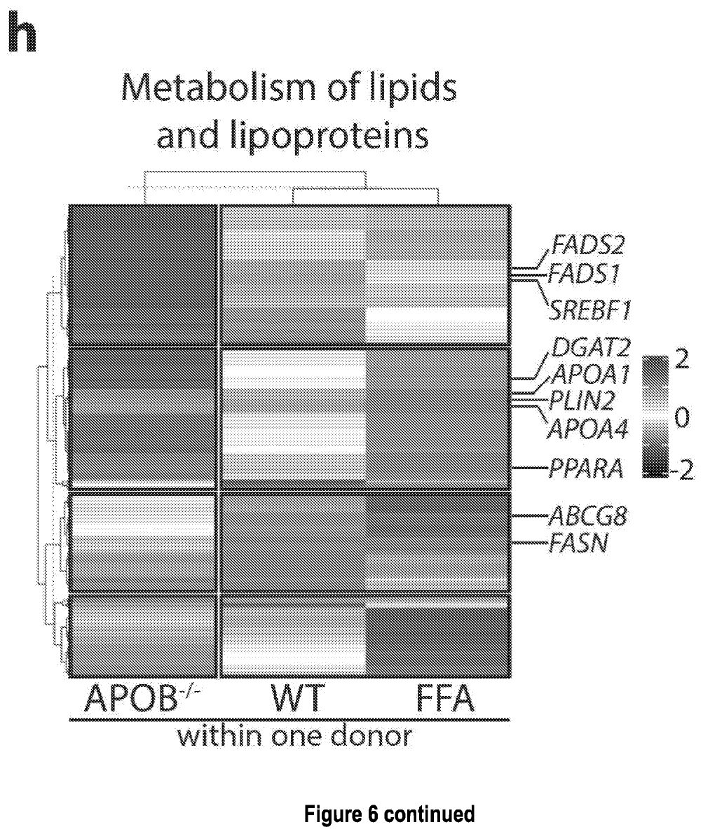

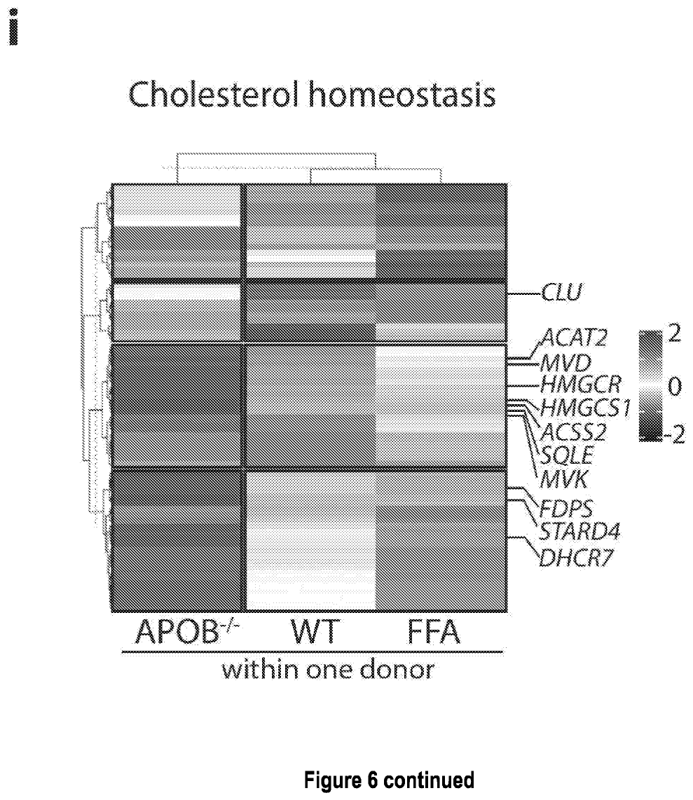

In a complementary approach it was aimed to generate steatosis organoids resembling dietary causes. To this end, wild type organoids were exposed to a concentrated free fatty acid (FFA) mixture consisting of oleic acid and palmitic acid, the two most abundant circulating FFAs (FIG. 5a). Upon titration of the FFA mixture, a similar degree of steatosis in the FFA-loaded organoids was reached as compared to APOB- or MTTP-mutant organoids, as assessed by lipid staining (FIG. 5b). Quantifications revealed a similar lipid droplet area coverage (ca. 30%) with more, yet smaller lipid droplets (FIG. 5c-d). Next FFA-loaded organoids from two different donors were exposed to bulk RNA sequencing and a significant transcriptomic rewiring (FIG. 5e) was observed, with 1552 genes differentially expressed compared to donor-matched wild type organoids (FIG. 5f). GO-term analysis on the DEGs revealed an impact on many cellular processes, including—as expected—lipid metabolic processes but also processes linked to cell cycle and replication (FIG. 6a). DisGeNET analysis highlighted resemblance with NAFLD, but also more aggressive forms of liver dysfunction including hepatocarcinogenesis and fibrosis (FIG. 6b).

Evaluation of the DEGs related to lipid metabolism revealed a diverse set of cellular responses (FIG. 5f-g). A strong induction of genes involved in mitochondrial (e.g. CPT1A, HADHA, HADHB) and peroxisomal (e.g. ACOX1, ACAA1) fatty acid β-oxidation was noted. In addition, the key ketogenic gene HMGCS2 was strongly induced, pointing towards enhanced ketogenesis as a way to dispose of lipids. Induction of many APO genes (e.g. APOA1, APOA2, APOA4, APOM) was also noted. Apoa1^{-/-} mice have reduced hepatic secretion of TAG-rich VLDL particles (Karavia et al., 2012), while Apom^{-/-} mice develop spontaneous steatosis linked to defects in autophagy (Zhang et al., 2018), emphasizing important roles of APO proteins in balancing hepatic TAG homeostasis. It was noted that many of the observed DEGs are under control of peroxisome proliferator-activated receptor alpha (PPARα), (e.g. CPT1A, PDK4, etc.), and indeed a strong general PPAR signaling response (FIG. 6c) was observed.

A palmitate derivative (C16:0/C18:1-GPC) was previously identified as endogenous PPARα ligand in mouse liver (Chakravarthy et al., 2009). It is likely that cellular conversion of the FFAs present in the fat mixture similarly yields an endogenous PPAR ligand. While most lipid metabolism-related DEGs were upregulated, some genes were downregulated. The latter included FASN, a key hub involved in TAG synthesis, as well as the fructose metabolism-related genes KHK and HK2. Additionally, induction of genes involved in fibrogenesis and activation of hepatic stellate cells and macrophages (e.g. TGFB1, HRG, CTGF, TIMP1) was observed, this was interpreted as signals from the hepatocyte that can promote progression towards NASH in vivo (FIG. 6d). In addition, as highlighted by GO term analysis, key genes related to cell cycle and DNA replication were among the DEGs (FIG. 6e), including strong repression of Wnt target genes (FIG. 6f), collectively suggesting an impairment in hepatocyte proliferation.

How the DEGs observed in APOB^{-/-} organoids compared with the DEGs found in FFA-loaded wild type organoids was studied. Only, a small set of genes (120 total) was conserved between both conditions, yet—remarkably—the majority of these with opposing directionality (FIG. 5h, FIG. 6g). DEGs related to lipid metabolism in the two different models (FIG. 5i) were systematically compared. The core de novo lipogenesis gene FASN was downregulated in both systems. Conserved downregulation of genes associated with glycolysis: PKLR, encoding pyruvate kinase that catalyzes the final step of the glycolytic pathway (irreversible conversion of phosphoenolpyruvate to pyruvate and ATP), and TKT, encoding transketolase, active in the pentose phosphate pathway where it channels sugar phosphates to glycolysis were also observed. Downregulation of HMGA1, encoding a chromatin binding protein, previously implicated in insulin resistance and diabetes (Foti et al., 2005) was also noted.

A number of common DEGs were regulated in opposing directions, including FABP1, MOGAT3, FADS6, SDC1, all downregulated in APOB^{-/-} organoids but upregulated in FFA-loaded wild type organoids. The majority of DEGs were, however, unique to each system: while APOB^{-/-} organoids collectively downregulated key genes involved in lipogenesis and cholesterol biosynthesis, FFA-loaded wild type organoids instead predominantly upregulated genes aimed at lipid digestion. Visualization of the expression of genes involved in triglyceride and cholesterol metabolism and homeostasis upon FFA exposure or loss of ApoB in organoids from the same donor highlights these divergent responses (FIG. 6h-i). Thus, depending on the steatosis trigger, hepatocyte responses are completely different, though presenting identical steatosis phenotypes.

### Unified Responses of Genetic and Diet-Driven Steatosis Organoids Towards Putative Anti-NAFLD Drugs

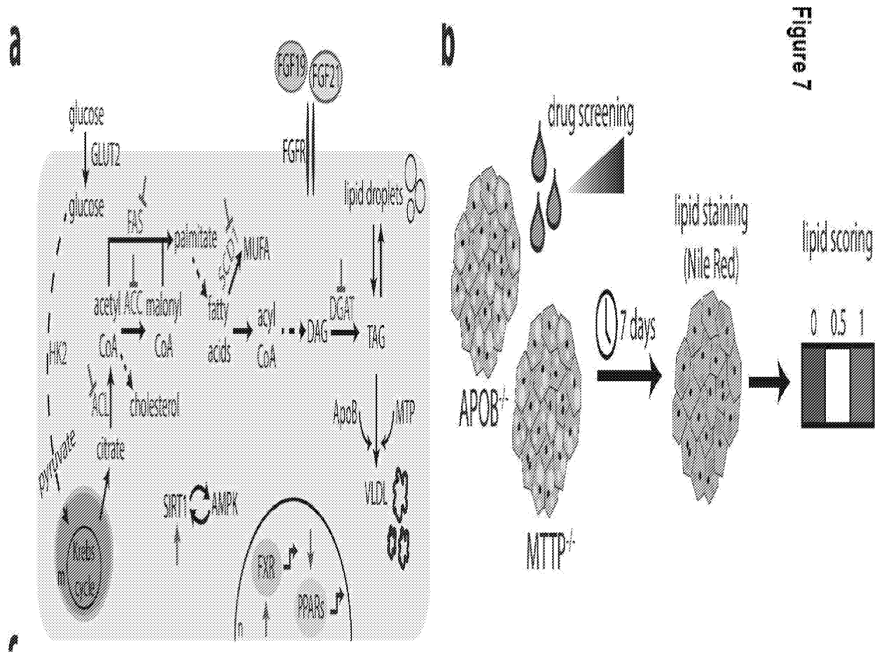

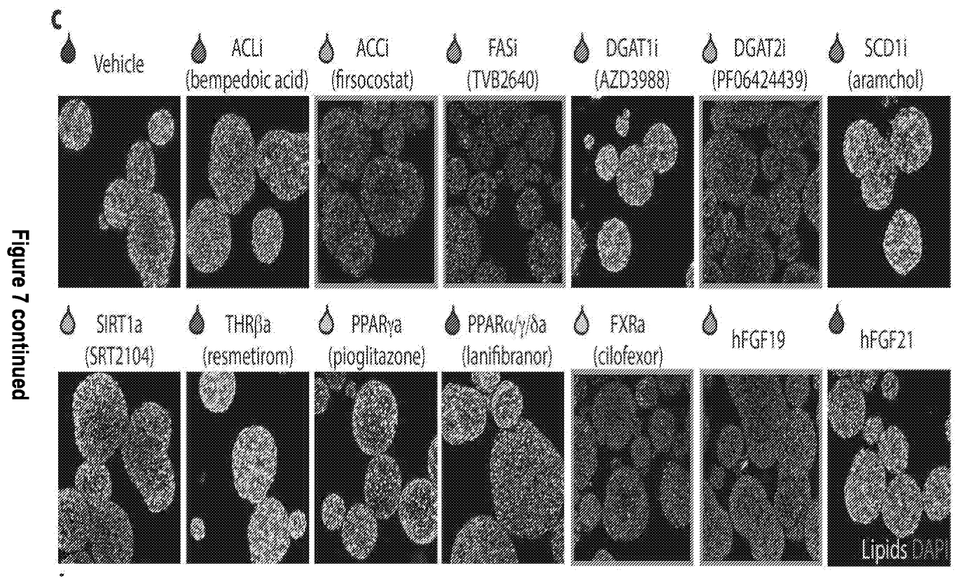

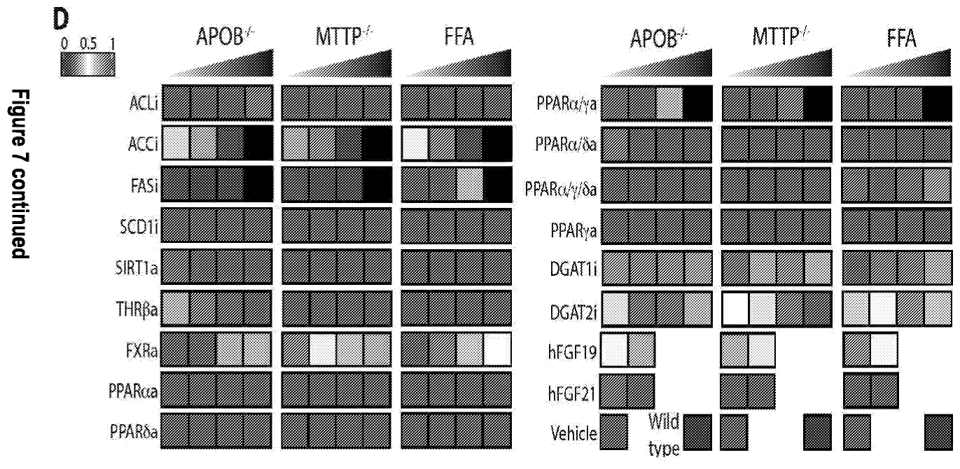

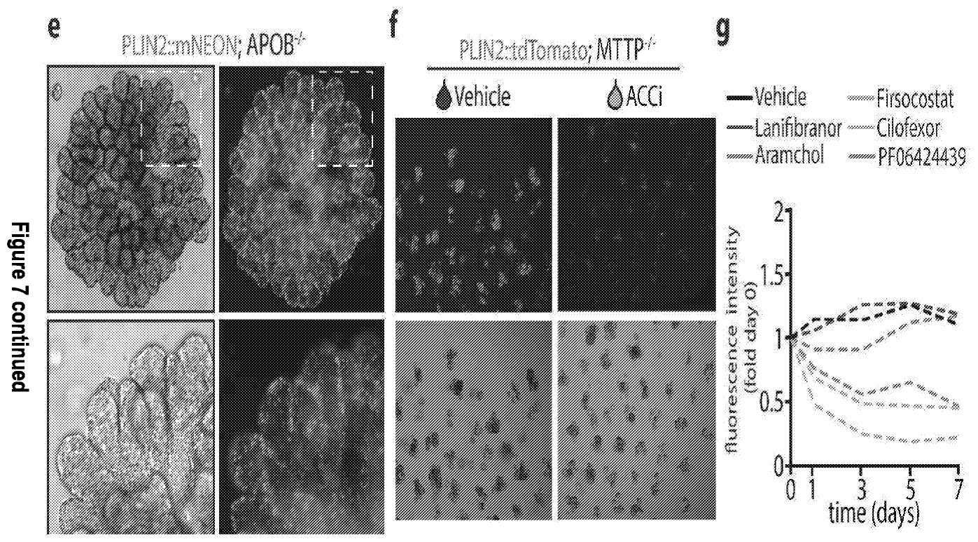

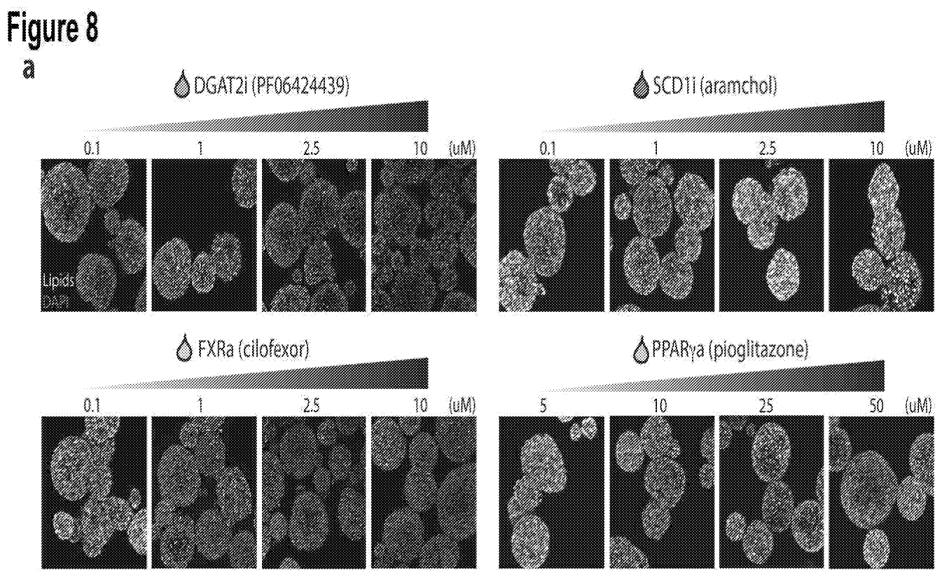

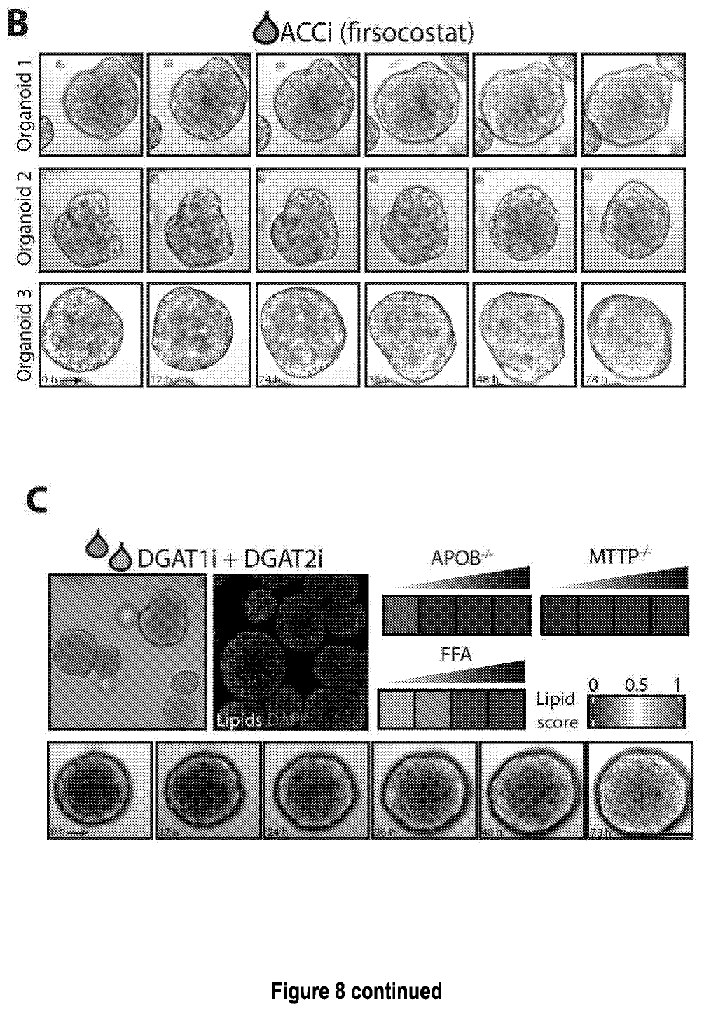

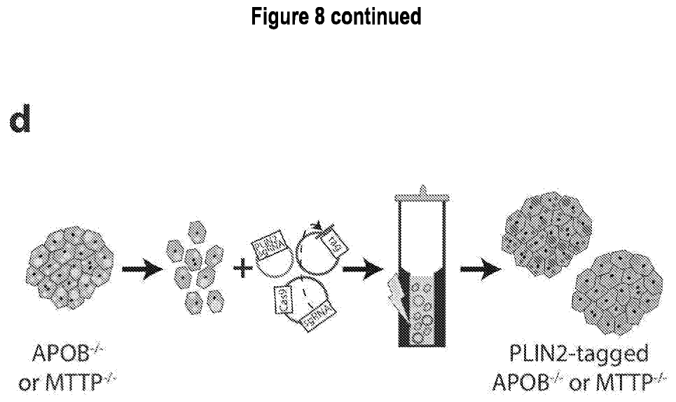

Anti-NAFLD drugs remain highly sought after, and multiple major metabolic targets are currently under evaluation (FIG. 7a). It was questioned how the genetic and dietary steatosis models would respond to an array of 17 putative drugs highlighted for NAFLD therapy (FIG. 7b, Table 2). A lipid scoring system was established that integrates the fluorescence intensity and area coverage of the lipid droplets within the organoid to score drug effectiveness. A selected set of drugs markedly reduced the steatosis phenotype of APOB^{-/-} organoids (FIG. 7c), most displaying a clear dose-dependent effect (FIG. 8a). This could be visualized in in real-time by live imaging the organoids during drug treatment (FIG. 8b).

APOB^{-/-} and MTTP^{-/-} organoids reassuringly displayed identical drug responses. Notably, FFA-loaded wild type organoids responded too in a near-identical manner, though it generally appeared more difficult to reduce the extent of lipid accumulation in this model (FIG. 7d). In both models, inhibition of ACC, FAS, and DGAT2 (all enzymes acting along the de novo lipogenesis pathway) worked well. Inhibition of DGAT1 was much less effective. While DGAT1 primarily esterifies exogenous fatty acids, DGAT2 preferentially incorporates de novo lipogenesis-derived fatty acids into TAGs (Qi et al., 2012). Of note, combined treatment with both DGAT1 and DGAT2 inhibitors could completely revert steatosis phenotypes in both models (FIG. 8c). FXR activation and recombinant hFGF19 (but not hFGF21) also exerted marked steatosis-reducing effects. Instead, selective, dual, or pan PPAR agonists, as well as thyroid receptor beta (THRβ) agonism, ATP citrate lyase (ACL) inhibition, and sirtuin 1 (SIRT1) activation appeared ineffective at resolving the steatosis phenotype of both genetic and diet-driven models. Thus, despite major divergent transcriptomic responses, both steatosis models are unified in their drug response.

Perilipins coat lipid droplet particles (Itabe et al., 2017). It was hypothesized that fluorescently labeling endogenous PLIN2, the most abundantly expressed perilipin in liver, would enable to establish an image-based steatosis drug screening system. Previously established non-homologous end joining (NHEJ)-mediated gene knock-in strategy (Artegiani et al., 2020) was used to tag the endogenous locus of PLIN2 (FIG. 8d). It was possible to efficiently tag PLIN2 with tdTomato or mNEON in both APOB^{-/-} and MTTP^{-/-} organoid lines (FIG. 8e). The fluorescent signal derived from the reporters appeared with the typical lipid droplet morphology (FIG. 7e). Counterstaining of the PLIN2 reporters with a lipid droplet dye demonstrated faithful tagging of lipid droplets (FIG. 8f) and confirmed that tagging of PLIN2 did not affect the steatosis phenotype of the organoids (FIG. 8g). As a pilot, the organoids were treated with ACCi which, as expected, reduced the endogenous fluorescent signal (FIG. 7f). Next the organoids were subjected to a set of 5 previously screened drugs (3 positive, 2 negative) to assess the robustness of the system. Quantification of the fluorescent signal over a timeframe of 7 days visualized variable drug dynamics with time and demonstrated an identical classification of effective drugs (FIG. 7g, FIG. 8h), thus establishing PLIN2-reporter APOB^{-/-} or MTTP^{-/-} organoids as real-time lipid reporter systems.

### Conserved and Unique Modes of Action of Anti-NAFLD Drugs

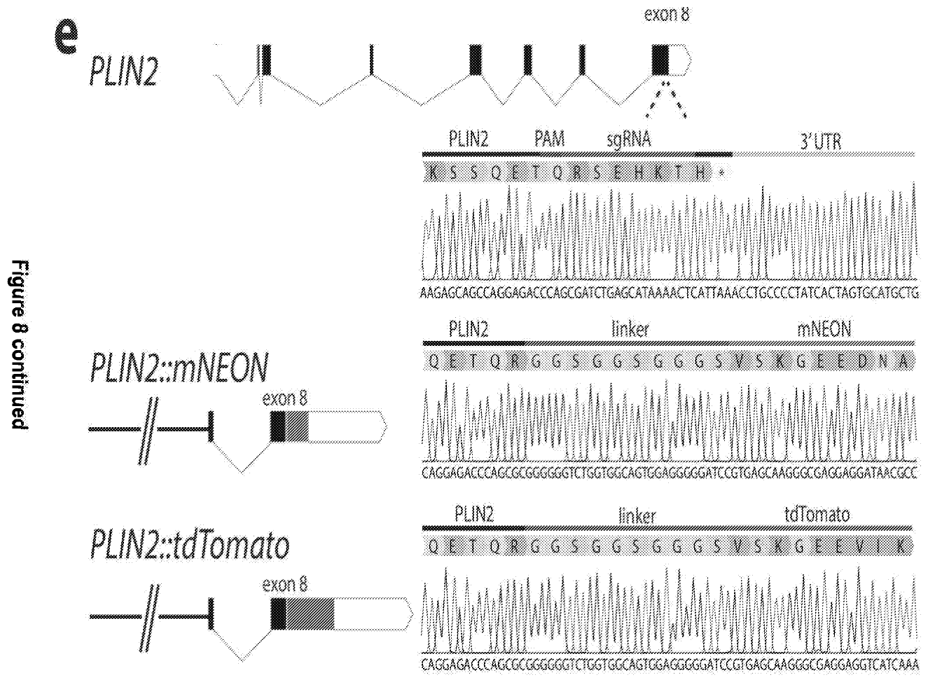

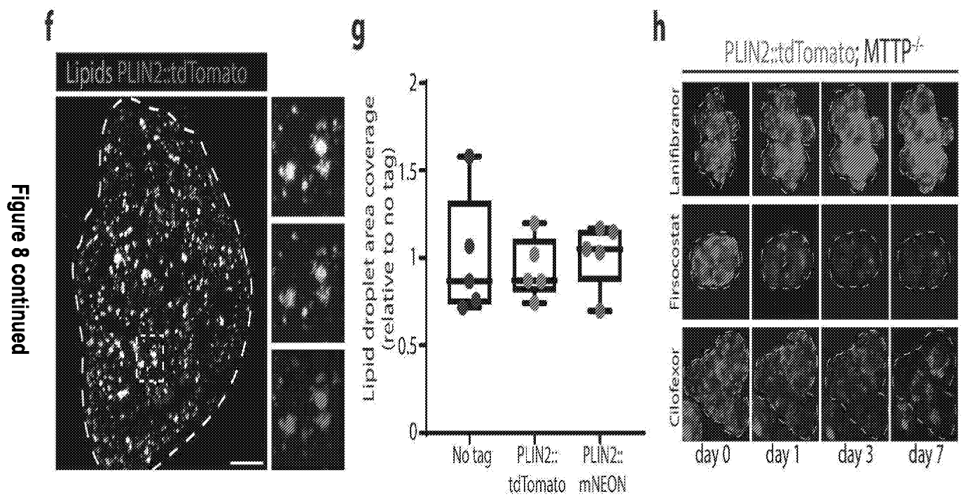

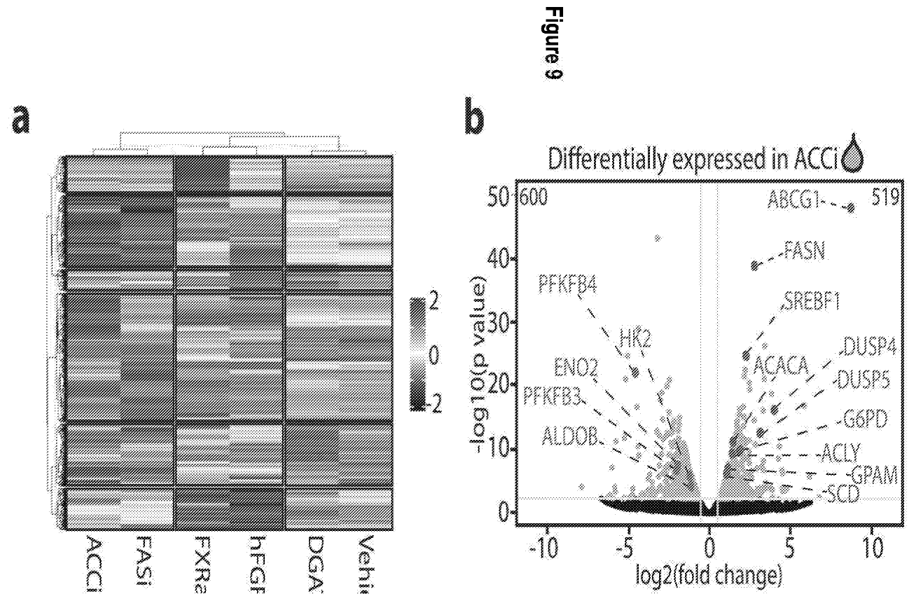

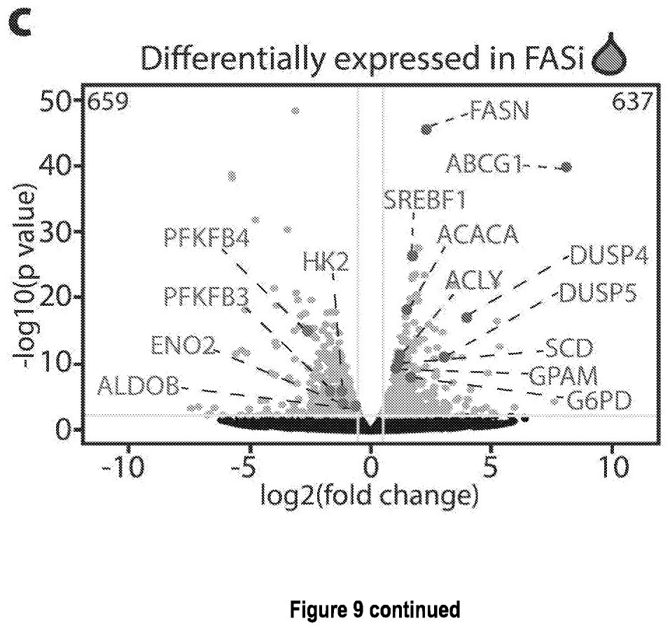

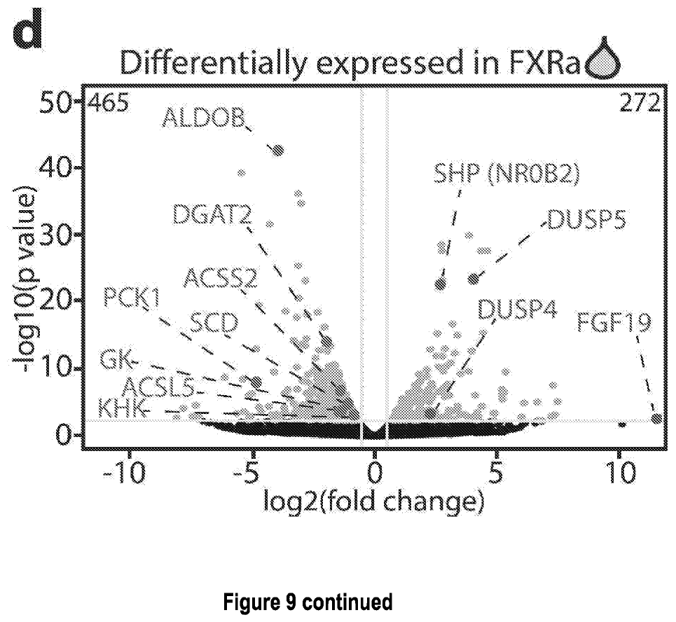

APOB^{-/-} organoids treated with DGAT2i, FASi, ACCi, FXRa, hFGF19, or vehicle were subjected to bulk RNA-sequencing to obtain mechanistic understanding of how cells respond to these drugs. Differential gene expression analysis revealed striking drug-specific patterns (FIG. 9a). FASi and ACCi induced very similar transcriptomic changes, as did-independently-FXRa and hFGF19, while DGAT2i appeared transcriptomically identical to vehicle-treated organoids.

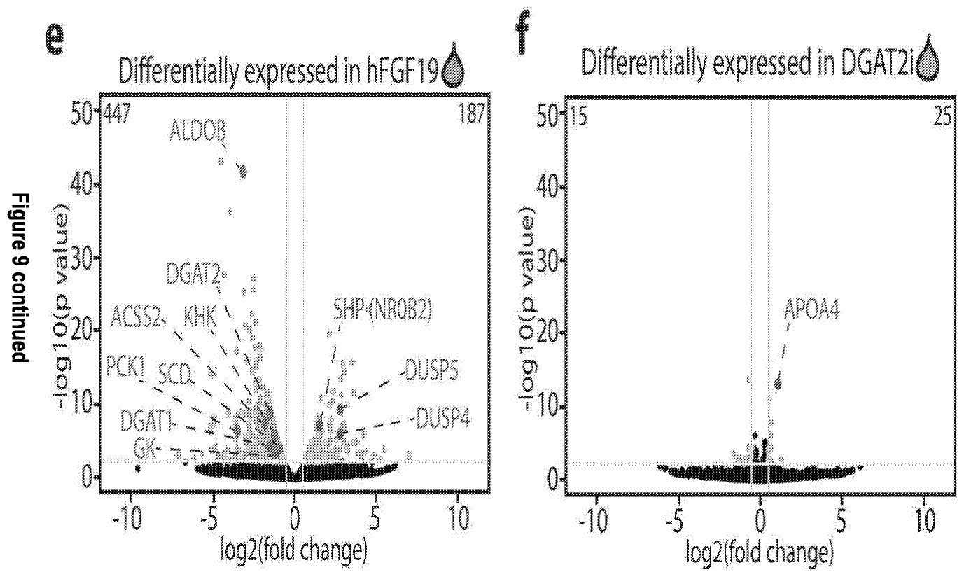

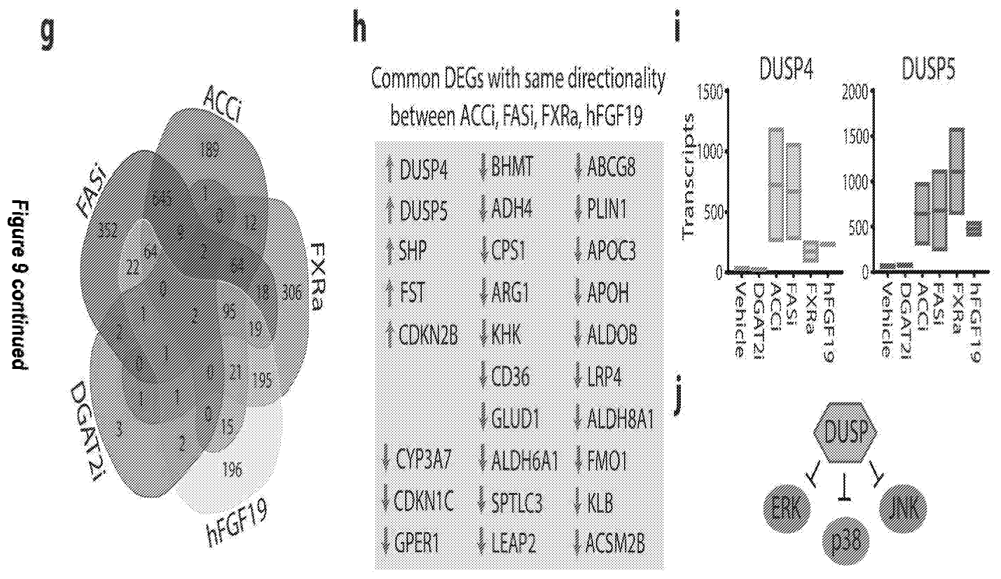

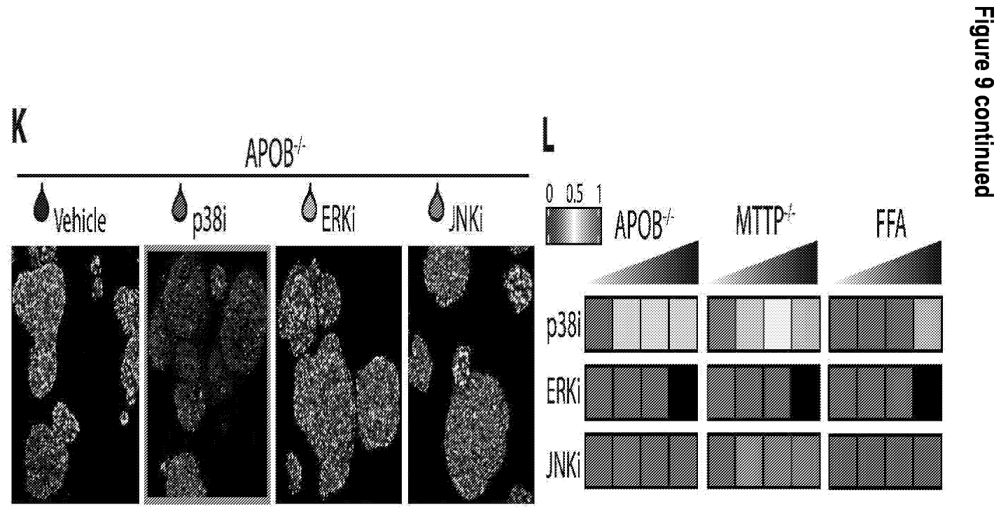

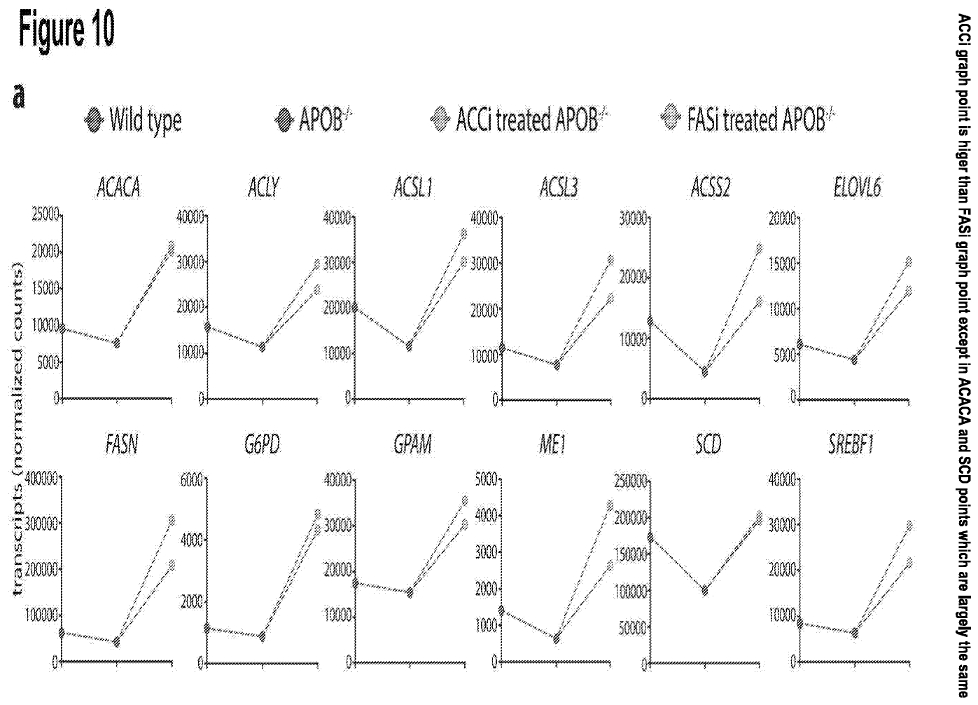

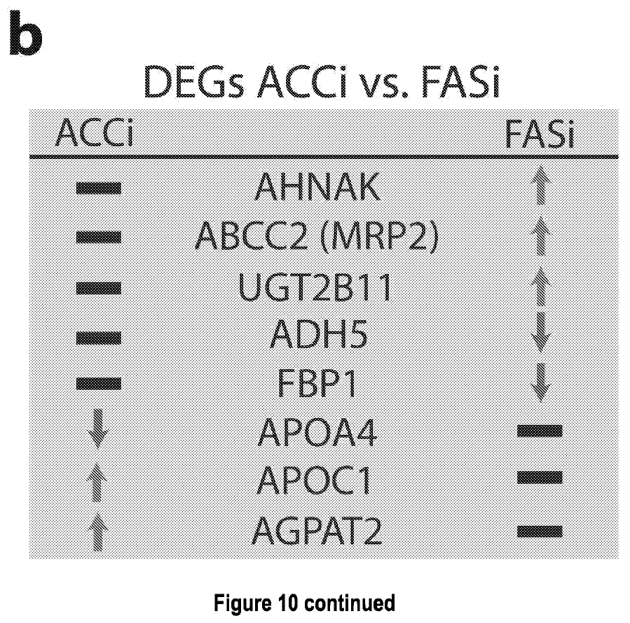

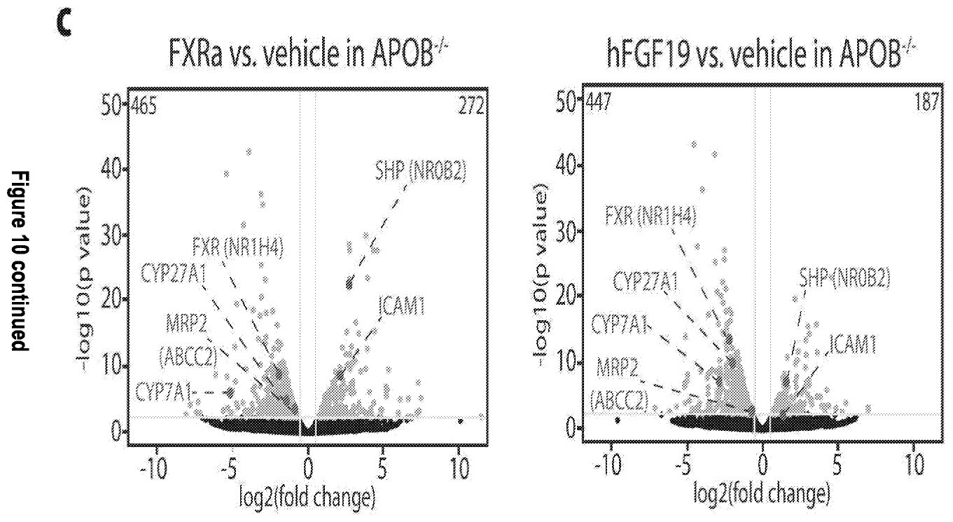

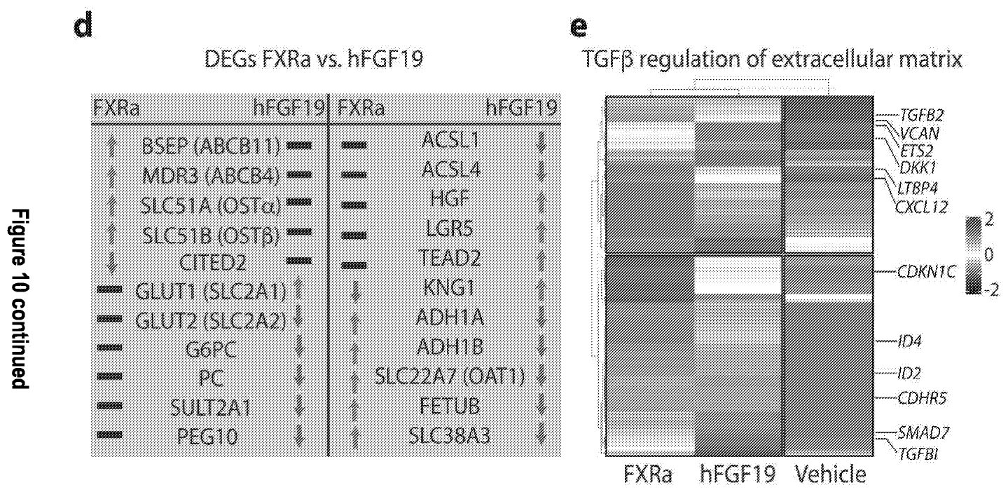

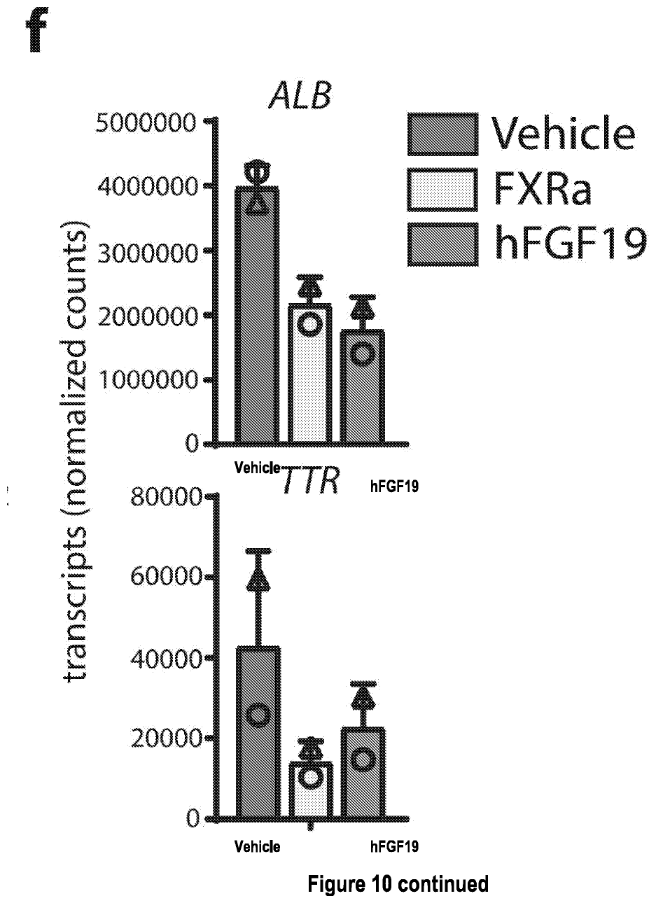

Individual drug actions were then focused on. ACCi- and FASi-treated organoids downregulated several key genes involved in glycolysis (ALDOB, HK2, ENO2, PFKFB3/4). Unexpectedly, increased expression of many lipogenic genes (ACACA, FASN, SREBF1, etc.) (FIG. 9b-c) was noted. Since APOB^{-/-} organoids intrinsically downregulate many of these genes, their expression was also benchmarked to wild type organoids, and it was found that the net induction to exceed the wild type expression levels (FIG. 10a). We observed very few unique DEGs in either ACCi or FASi conditions alone (FIG. 10b), pointing towards identical mechanisms of drug action.

FXRa and hFGF19 displayed related transcriptomic profiles (FIG. 9d-e). FXRa exclusively strongly upregulated FGF19 expression (which hFGF19 did not), thus FXRa not only stimulates secretion of FGF19 from the gut to the liver, but also exerts the same function directly in the liver, as previously suggested with regard to bile acid homeostasis (Song et al., 2009). Typical bile acid synthesis-related FXR target genes both in FXRa- and hFGF19-treated organoids were noted (e.g. downregulation of CYP7A1 and CYP27A1 and induction of NR0B2 (SHP)) (FIG. 10c). A handful of DEGs were exclusively found in either condition, including FXRa-related induction of several transporters such as BSEP (ABCB11) (FIG. 10d). Among the common DEGs, we noted both FXRa and hFGF19 to repress various de novo lipogenesis-related genes, including DGAT2, ACSS2, and GPAM, providing possible explanations for their steatosis reducing effects. Of note, for both drugs gene induction patterns suggestive of TGFβ-related extracellular matrix remodeling (FIG. 10e), as well as downregulation of key hepatocyte markers (ALB, TTR) (FIG. 10f) were observed. DGAT2i caused very few transcriptomic changes appearing identical to vehicle-treated APOB^{-/-} organoids suggesting a very “clean”, posttranslational effect of DGAT2i on reducing steatosis (FIG. 9f).

Next the orchestrated responses induced by the different drugs were focused on and found a number of DEGs to be conserved across all treatments (excluding DGAT2i) (FIG. 9g-h). In particular, induction of DUSP4 and DUSP5 was noted (with baseline expression near zero, but induced upon drug treatment) (FIG. 9i). DUSPs (dual-specificity phosphatases) regulate MAPK signaling pathway activity, including ERK, JNK, and p38 (Caunt and Keyse, 2013) (FIG. 9j). It was questioned whether interference with these pathways using specific small molecules would have an effect on the steatosis phenotypes of APOB^{-/-} and MTTP^{-/-} organoids. Treatment with ERKi and JNKi did not alter the steatosis phenotype, however treatment with p38i reduced the steatosis phenotype of both APOB^{-/-} and MTTP^{-/-} organoids (FIG. 9k). Lipid score analysis revealed a reduction (FIG. 9I). These MAPK inhibitors in FFA-loaded wild type organoids were also tested.

### Identification of Lipid Homeostasis Mediators Through CRISPR Screening

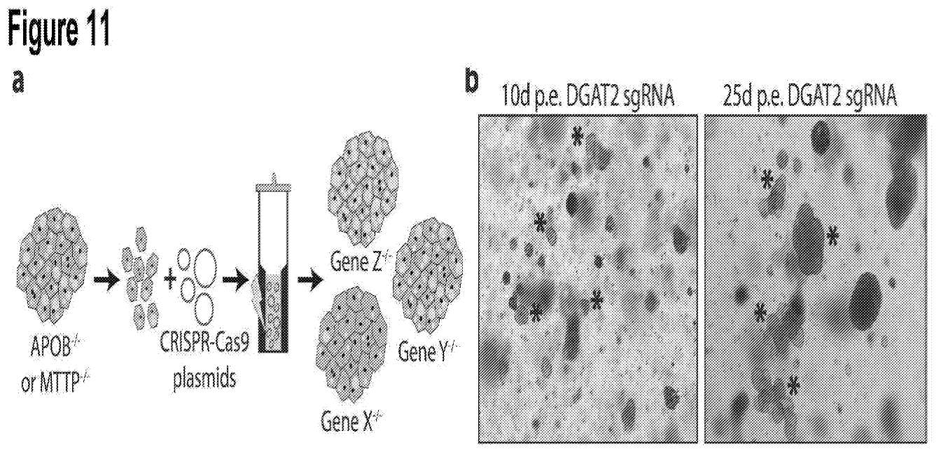

It was noticed that some of the identified metabolic targets (e.g. ACC, FAS, DGAT2) effective at reducing steatosis were already highlighted in the DEG list of APOB^{-/-} organoids (FIG. 5d). This observation suggested that this list could contain additional genes relevant for steatosis modification. To this end, a small-scale rational CRISPR screen using our APOB^{-/-} or MTTP^{-/-} organoids was performed, individually knocking out selected genes and observing the phenotype of the outgrowing organoids (FIG. 11a, Table 3).

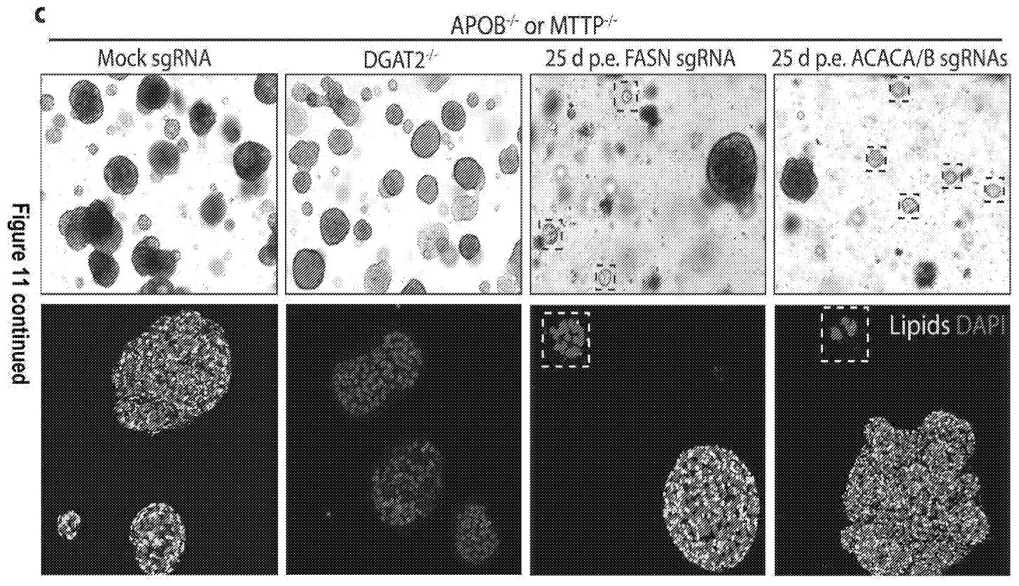

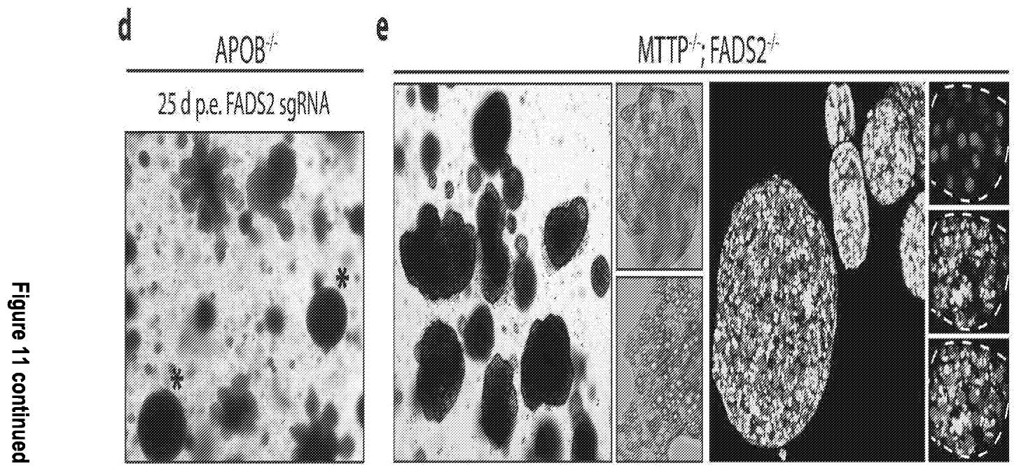

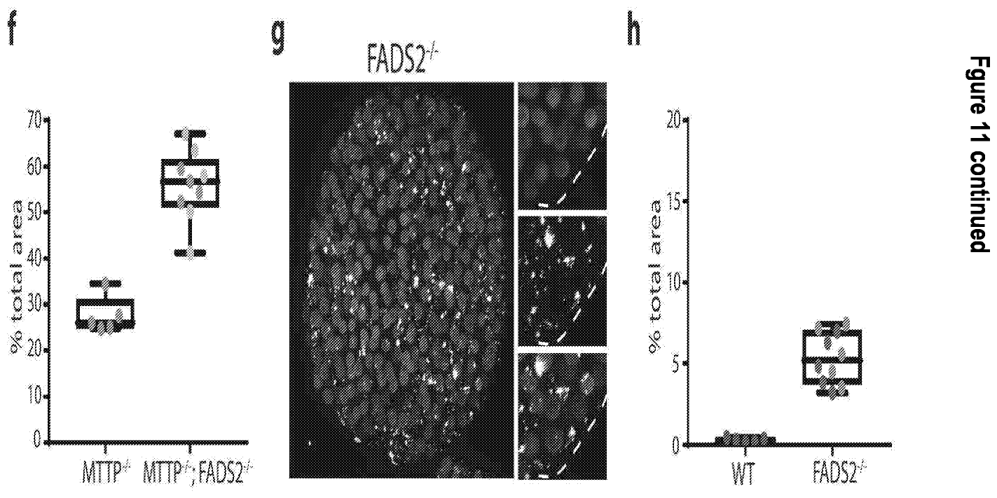

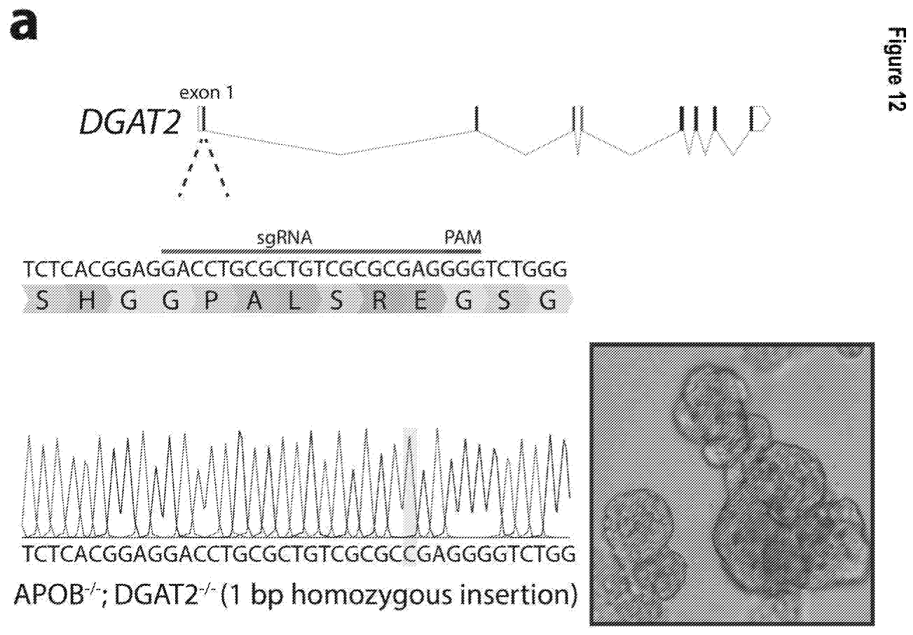

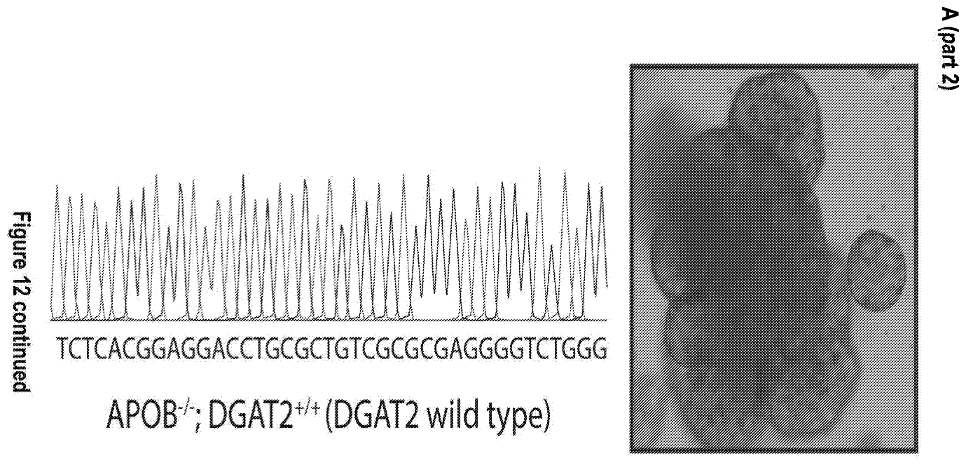

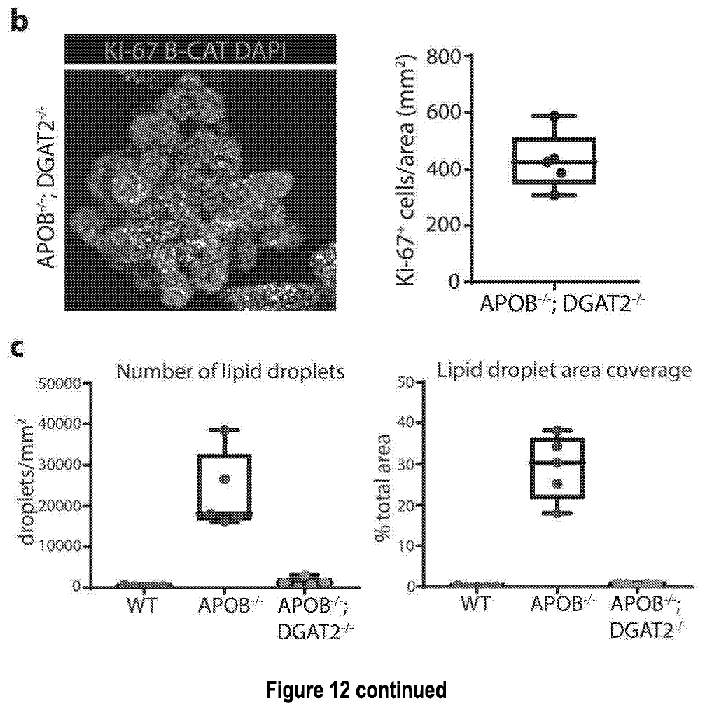

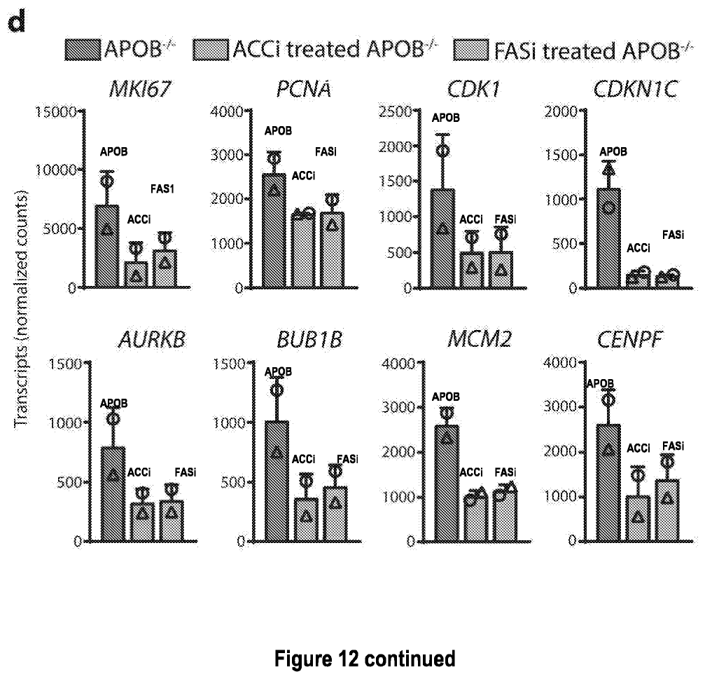

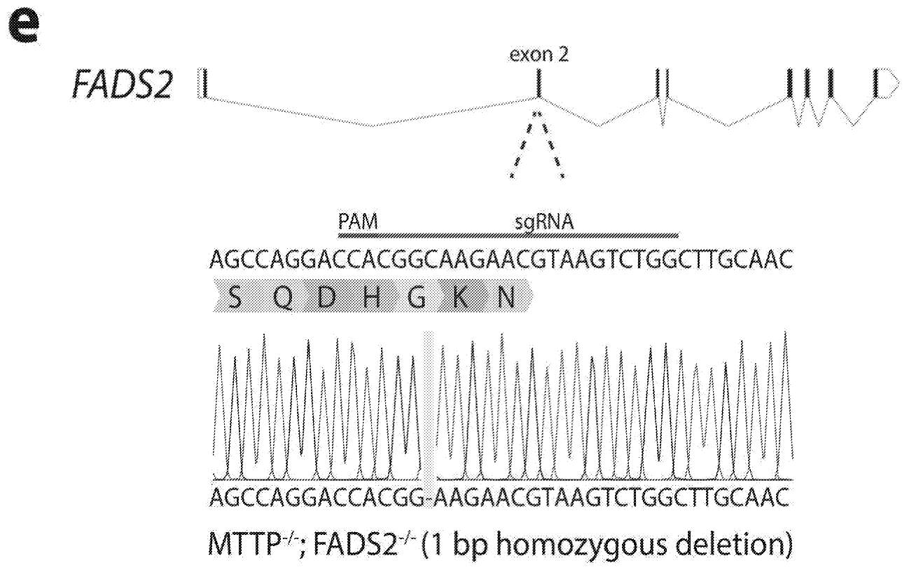

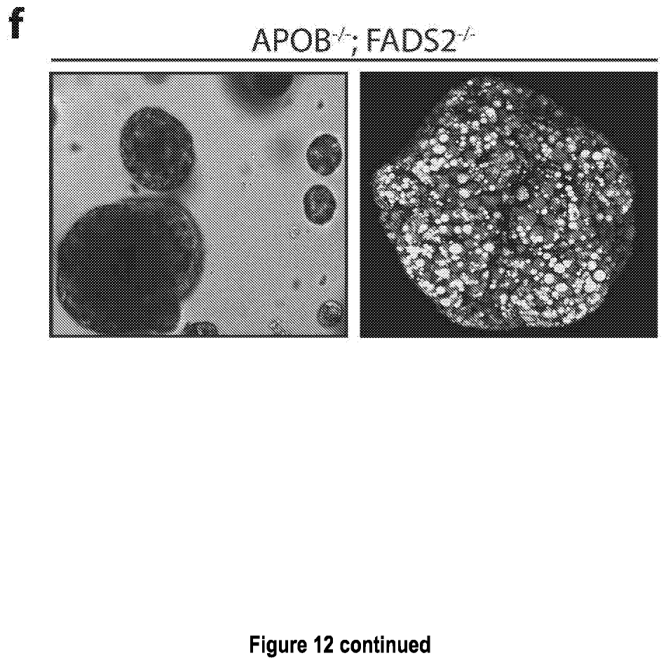

The effects of genetic ablation of the confirmed targets, ACC (ACACA/ACACB), FAS (FASN), and DGAT2 (DGAT2) was first focused on. During the outgrowth of Cas9/DGAT2 sgRNA-targeted APOB^{-/-} or MTTP^{-/-} organoids, phenotypic differences between individual outgrowing organoids were immediately noticed. Some organoids presented with the typical steatosis phenotype, while other organoids appeared fat-free (FIG. 11b). These latter organoids harbored homozygous DGAT2 mutations, while, as expected, lines derived from outgrowing steatosis-bearing organoids carried no DGAT2 mutations (FIG. 11a). The DGAT2^{-/-} clonal lines in the background of APOB^{-/-} or MTTP^{-/-} were viable and proliferative (FIG. 11b). Lipid staining and quantifications revealed a near-absence of lipid droplets, reverting back to levels found in wild type organoids (FIG. 11c, FIG. 12c).

When evaluating the outgrowth of Cas9-ACACA+ACACB sgRNA- or Cas9-FASN sgRNA-targeted organoids, the appearance of several small fat-free organoids that were never present in mock transfections with Cas9 and a non-human sgRNA were noted (FIG. 11c). These fat-free organoids were composed of just a few cells and rapidly seized to proliferate, making it impossible to derive clonal lines from these organoids. Thus, genetic ablation of FAS or ACC appeared not compatible with proliferation of hepatocytes. When re-evaluating transcriptomic data from ACCi- or FASi-treated APOB^{-/-} organoids, downregulation of many genes important for cell proliferation and DNA synthesis was noted (FIG. 12d).

Among the screened genes, phenotypic differences upon outgrowth of cells transfected with Cas9-FADS2 sgRNA were noted (FIG. 11d). FADS2^{-/-} clonal lines in the background of MTTP^{-/-} could be derived (FIG. 12e), which presented as much darker—more lipid containing—organoids compared to FADS2^{+/+} counterparts (FIG. 11e). Similar results were obtained when generating FADS2^{-/-} lines in an APOB^{-/-} background (FIG. 12f).

Quantification of the area covered by the lipid droplets in these organoids indicated a steatosis level surpassing 50% (FIG. 11f). Knock-out of FADS2 in wild type organoids also induced a phenotypic switch: these organoids spontaneously accumulated lipid droplets (FIG. 11g), which covered 5% of the organoid area (FIG. 11h). FADS2 (fatty acid desaturase 2) is a delta-6 desaturase that mediates the biosynthesis of long-chain PUFAs from the essential PUFAs linolenic and linoleic acid. Thus, intact PUFA metabolism and FADS2 activity is important in balancing steatosis levels and lipid homeostasis within hepatocytes.

### Role of PNPLA3 and the I148M Variant in Steatosis

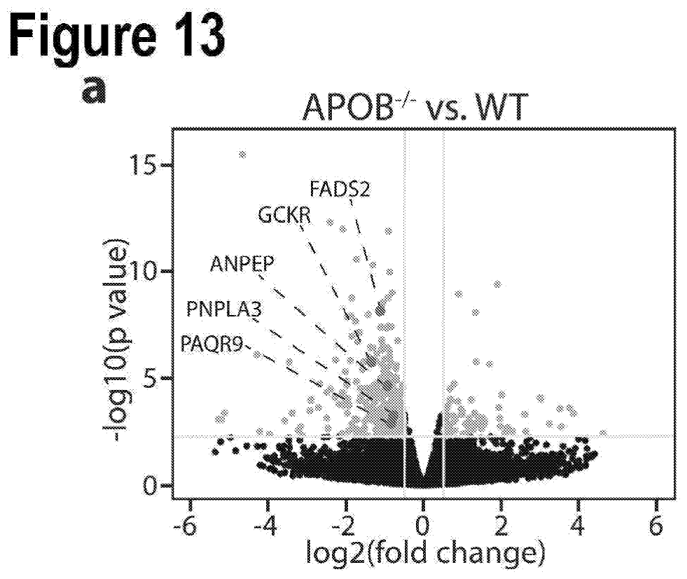

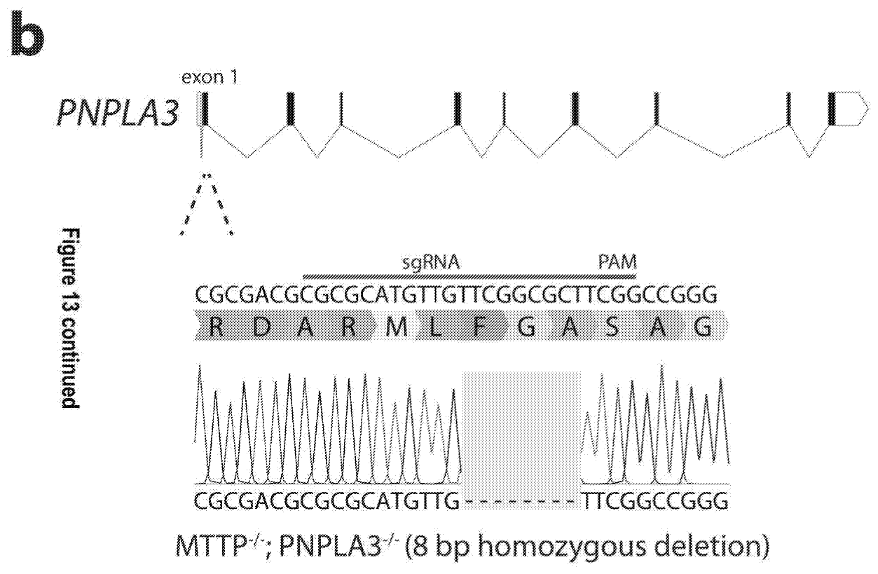

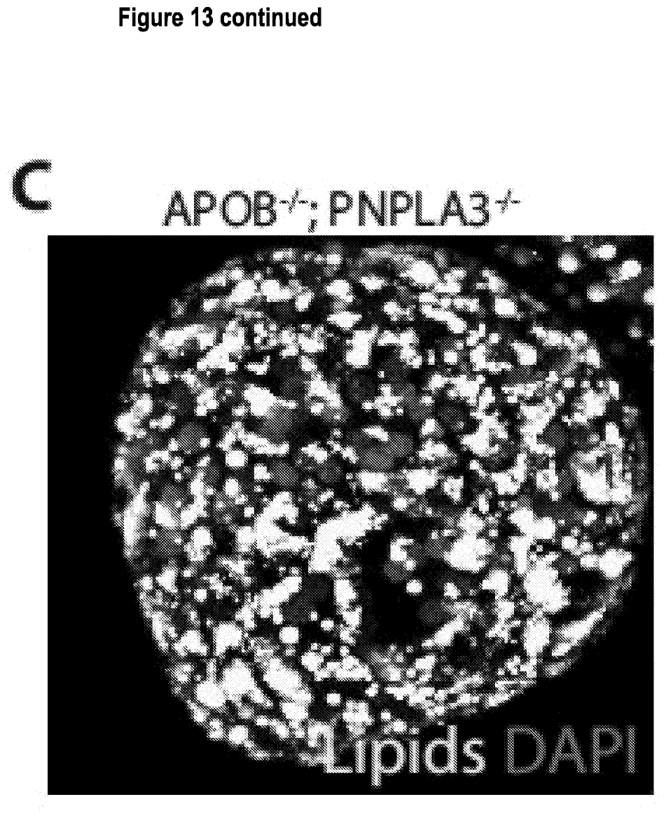

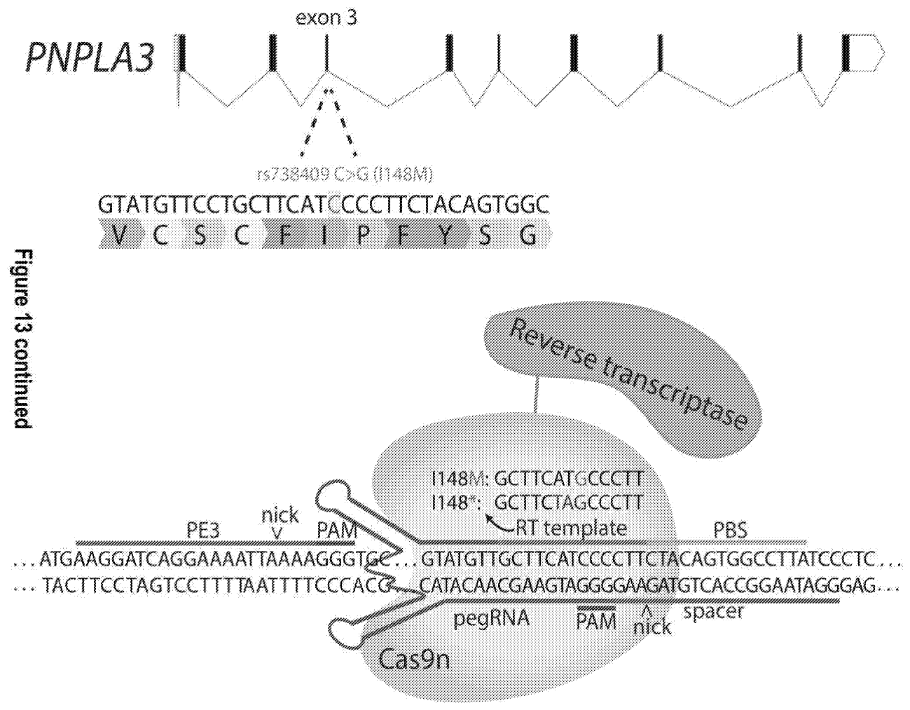

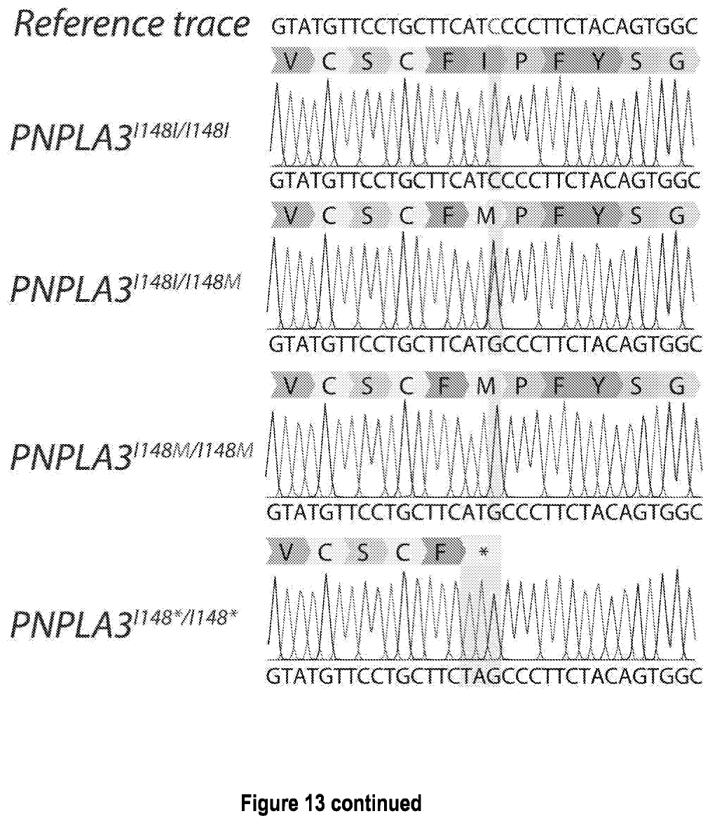

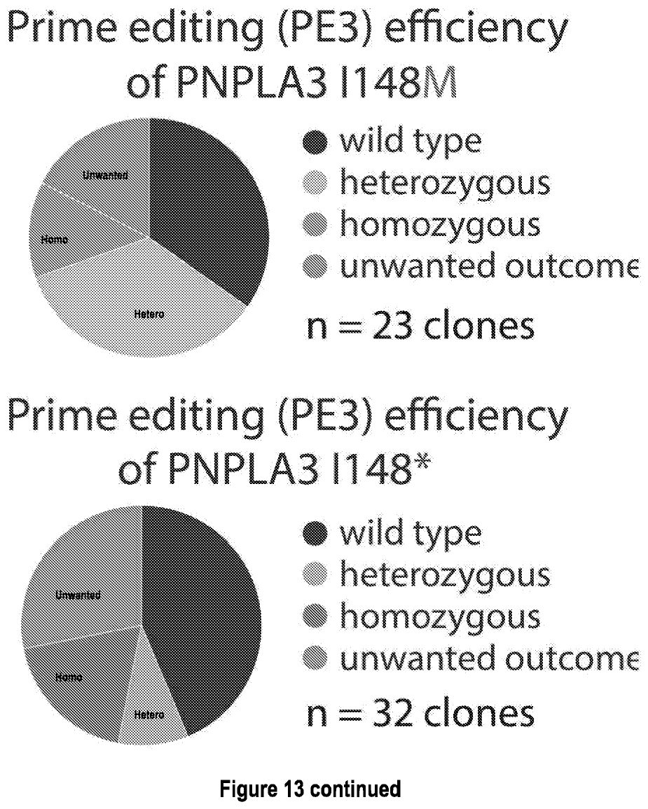

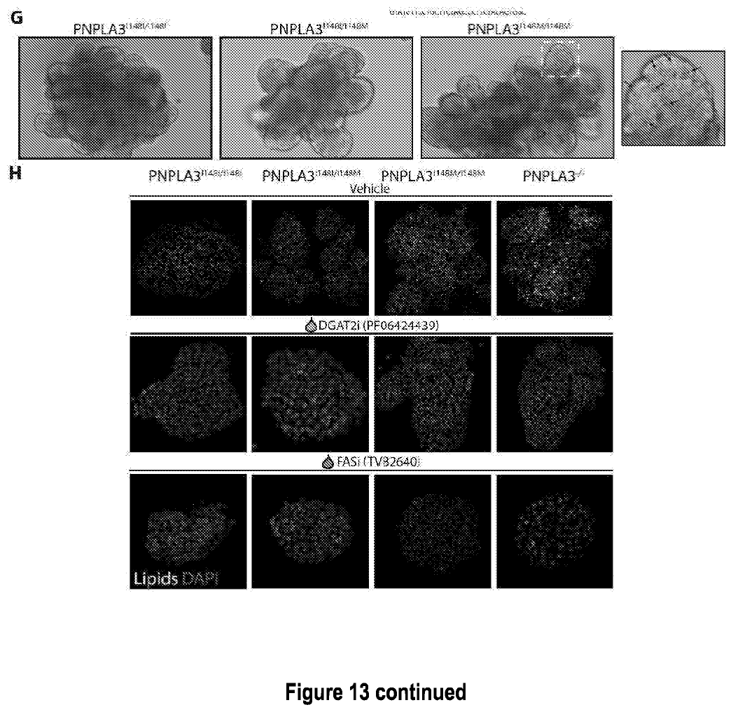

The APOB^{-/-} DEG list contained several genes that have emerged from genome-wide association studies as risk factors for NAFLD, including GCKR and PNPLA3 (FIG. 13a). It was decided to study the role of PNPLA3 in steatosis by generating sequential gene knock-outs (FIG. 13b).

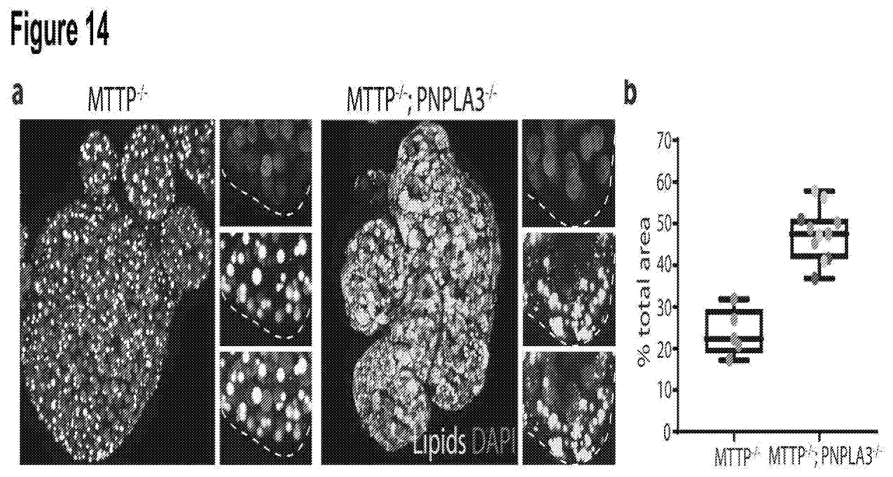

PNPLA3 knock-out markedly aggravated the phenotype of both APOB^{-/-} and MTTP^{-/-} organoids (FIG. 14a, FIG. 13c), reaching a lipid droplet area coverage of near 50% (FIG. 14b).

The consequences of PNPLA3 knock-out in wild type organoids was investigated. In contrast to Pnpla3 knock-out mice that do not show any steatosis phenotype (Basantani et al., 2011; Chen et al., 2010), wild type human hepatocyte organoids spontaneously accumulated lipid droplets when PNPLA3 was mutated, often visible as small droplets residing at the cell border (FIG. 14c). Lipid droplets covered 8% of the organoid, a degree similar to grade 1 steatosis (FIG. 14d).

The rs738409 polymorphism in PNPLA3, encoding the I148M variant, represents the strongest known genetic association with NAFLD (Romeo et al., 2008). To address the role of this SNP in both a human and isogenic setting, prime editing (Anzalone et al., 2019) was employed to engineer human hepatocyte organoids (genetic background: PNPLA3^{I148I/I148I}) to harbor PNPLA3 I148M in both heterozygous and homozygous form. Homozygous PNPLA3 I148* organoids were also generated as an internal control (FIG. 13c). Organoids could be efficiently prime edited into the desired genotypes (FIG. 13d), with few unwanted outcomes (FIG. 13e).

Strikingly, organoids engineered to carry the PNPLA3 I148M variant in homozygous form (PNPLA3^{I148M/I148M}) spontaneously accumulated lipid droplets, while heterozygous variants (PNPLA3^{I148I/I148M}) accumulated much fewer droplets, indicating that the I148M variant impairs the homeostasis of lipids generated de novo (FIG. 14e, FIG. 13f). Lipid droplet area quantifications revealed that PNPLA3^{I148M/I148M} mutants approached PNPLA3^{-/-} steatosis levels (FIG. 14f).

The sensitivity of the different PNPLA3 mutant organoids towards a challenge with exogenously provided FFAs was studied. Both PNPLA3^{I148M/I148M} and PNPLA3^{/148M/1148M} organoids accumulated lipids at lower FFA concentrations than PNPLA3^{I148M/I148I} organoids (FIG. 14g-h), indicating that the I148M variant also confers susceptibility to steatosis when challenged by circulating FFAs. To probe potential treatment options, it was found that the intrinsic steatosis phenotype of the PNPLA3 mutant variants could be efficiently be reversed using, for example, the previously screened inhibitors of DGAT2 and FAS (FIG. 13g).

Finally, it was questioned whether if it was possible to genetically revert the I148M mutation back into I148I as a proof-of-concept strategy. To this end, the engineered heterozygous and homozygous I148M organoids were subjected to a second round of prime editing, this time using a pegRNA specifically targeting the DNA sequence of I148M. the same pegRNA on I148I organoids was also tested to assess gene-editing specificity.

### DISCUSSION

NAFLD is a complex and heterogeneous disease with high unmet medical need. Here, it is established and validate human hepatocyte organoids as model systems for the primary cause of NAFLD: steatosis. Liver organoids carrying APOB or MTTP mutations develop spontaneous steatosis driven by accumulation of lipids derived from de novo lipogenesis using carbohydrate sources from the—essentially fat-free—culture medium (FIGS. 1 and 3). In the other model, fatty liver organoids are generated through the accumulation of externally provided FFAs. In both models, steatosis results from an imbalance between hepatic lipid acquisition and lipid disposal.

Transcriptomic comparisons between these two types of fatty livers revealed remarkably distinct cellular responses (FIG. 5). APOB^{-/-} organoids collectively downregulated LXR-regulated genes involved in lipid homeostasis and cholesterol biosynthesis. Activation of LXR in liver results, amongst others, in de novo lipogenesis (Wang and Tontonoz, 2018). One of the established LXR ligands are oxysterols, which were found to be near absent in APOB^{-/-} organoids in contrast to wild type organoids. It was hypothesized that suppression of oxysterol synthesis (in order to limit LXR activation) constitutes a protective feedback mechanism of an APOB^{-/-} cell to minimize the generation of lipids. Instead, FFA-loaded wild type organoids displayed much broader transcriptomic responses, including the upregulation of genes to dispose of lipids through fatty acid oxidation and ketogenesis via the activation of PPARα. FFA-loaded wild type organoids induced widespread expression of key fibrinogenic genes and genes involved in activation of hepatic non-parenchymal cells known to induce progression towards NASH.

It is further shown how the transcriptomic profiles of these steatosis organoids can identify critical lipid homeostasis mediators and potentially novel therapeutic targets. To this end, APOB^{-/-} and MTTP^{-/-} provide real-time tunable fatty liver platforms to perform genetic screens (FIG. 11). An essential role for FADS2 that limits the degree of steatosis development in APOB^{-/-} or MTTP^{-/-} organoids was found, and loss of FADS2 induces spontaneous lipid accumulation in wild type organoids. SNPs in FADS2 have been linked with liver function (Chambers et al., 2011), and very recently with NAFLD (bioRxiv). Collectively, these indications point toward a critical role for FADS2, and as such PUFA metabolism, in NAFLD, highlighting FADS2 as a potential NAFLD target.

Heterogeneous but unified drug responses of the two steatosis models towards putative anti-NAFLD drugs was observed (FIG. 7). These screenings uncovered the ability of selected drugs, including inhibitors of ACC, FAS, DGAT2, as well as FXR agonism and recombinant hFGF19, to reduce or resolve steatosis in both models.

Obeticholic acid, an FXR agonist, was recently rejected by the US FDA for treatment of NASH as it failed to meet the liver fibrosis reduction endpoint (Mullard, 2020). Here, beneficial effects of FXR agonism is demonstrated in an earlier stage of NAFLD: reduction of steatosis in hepatocytes.

Other drugs, such as PPAR agonists, were ineffective in our steatosis models, suggesting these to not exert substantial beneficial effects directly on the hepatocyte level. While a strong endogenous PPAR activation response was observed upon fat loading in wild type organoids, additional drug-induced PPAR agonism appeared not beneficial. Indeed, PPAR agonists have been shown to exert beneficial effects in the context of resolving NASH through limiting macrophage and stellate cell activation in mice (Lefere et al., 2020). Strikingly, both steatosis models responded near-identical to the drug panel, despite their steatosis phenotype originating from two different causes. Effective drugs either have a direct target functioning in the de novo lipogenesis pathway (ACCi, FASi, DGAT2i), while transcriptomic evaluation of FXRa and hFGF19 suggested these drugs to also indirectly impact on this pathway. Thus, the data suggest that interfering with de novo lipogenesis is an effective way to collectively reduce steatosis.

Multiplexing drug screening with transcriptomics and CRISPR experiments revealed important drug traits (FIGS. 9 and 11). A counterintuitive upregulation of many lipogenic genes upon ACCi and FASi treatment was found, despite an effective reduction in the steatosis phenotype. These findings are in agreement with a previous report in hepatocyte-specific double Acaca/Acacb knock-out mice, presumably due to SREBP-1c induction caused by the drug-induced PUFA deficiency (Kim et al., 2017). Similar effects occurring upon inhibition of FAS are also reported here. Moreover, it was found that inhibition or genetic silencing of ACC or FAS severely impacted the proliferation capacity of APOB^{-/-} and MTTP^{-/-} hepatocytes, suggesting that therapeutical inhibition of these important metabolic proteins may negatively influence the liver regeneration capacity of patients. Instead, DGAT2-silenced organoids were viable and fat-free.

DGAT2i remarkably induced few transcriptomic changes, meaning that treated organoids maintain an “APOB^{-/-} transcriptome”, but do not revert back to wild type organoid transcriptomes despite resolving their steatosis phenotype. This more broadly questions the reversibility of steatosis phenotypes on a transcriptomic and functional level within a hepatocyte. FXR agonism was accompanied by induction of FGF19 expression, and the steatosis-reducing transcriptomic effects were remarkably similar to recombinant hFGF19 treatment, suggesting beneficial effects on NAFLD of FXR agonism to signal primarily through FGF19. Finally, by systematically analyzing the transcriptomic changes induced by different drugs, a common induction of DUSP4 and DUSP5, mediators of MAPK signaling were identified. In line, inhibition of p38 reduced the steatosis phenotype of a number of organoids is demonstrated.

Hepatic p38 signaling has been implicated in regulating gluconeogenesis (Cao et al., 2005). The findings now establish p38 as a potential NAFLD target.

Genetic risk factors confer clear susceptibility to NAFLD. Yet, the role of the most strongly linked gene, PNPLA3, remains somewhat enigmatic. Human PNPLA3 I148M carriers have lower VLDL secretion (Pirazzi et al., 2012), while TAG composition of VLDL particles show depletion of PUFAs (Luukkonen et al., 2019). Mouse experiments have not provided significant insights: Pnpla3 knock-out mice do not develop steatosis, even when maintained on a high-sucrose or high-fat diet (Basantani et al., 2011; Chen et al., 2010). Chronic overexpression of human PNPLA3^{I148M} but not wild type PNPLA3 in mouse liver causes steatosis when maintained on a chow or high-sucrose diet, but differences in liver fat levels disappear when mice are challenged with a high-fat diet (Li et al., 2012). Instead, Pnpla3^{I148M/I148M} knock-in mice, with physiological levels of protein expression, develop steatosis only when challenged with a high-sucrose diet (Smagris et al., 2015).

The function of PNPLA3 and its I148M variant in human hepatocytes has been directly addressed herein (FIG. 14). PNPLA3 knock-out aggravated the steatosis phenotype of APOB- and MTTP-mutant organoids, suggesting PNPLA3 to have a critical role in lipid homeostasis beyond impacting on VLDL secretion.

Secondly, PNPLA3 knock-out and—to a lesser extent—homozygous introduction of the I148M variant in wild type organoids led to spontaneous accumulation of lipid droplets in basal culture conditions, meaning those lipids to be derived from de novo lipogenesis. It was also found that the I148M variant also confers susceptibility to accumulate lipids derived from circulating FFAs, as I148M variant organoids displayed a heightened sensitivity towards an exogenous fat challenge, in line with a recent report using an iPSC-based model (Tilson et al., 2021). I148M heterozygotes have an intermediate phenotype. Since loss of PNPLA3 induces steatosis in human hepatocytes, this further challenges the recently proposed therapeutic strategy to inhibit or silence PNPLA3 to combat NAFLD (BasuRay et al., 2019; Linden et al., 2019). Instead, it was found that drug-mediated inhibition of de novo lipogenesis drugs can effectively reduce the intrinsic steatosis phenotype of PNPLA3-mutant organoids.

NAFLD covers a wide disease spectrum, where steatosis can further progress to the inflammatory subtype NASH. Here, many aspects of the first stage of the disease, i.e. simple steatosis occurring in human hepatocytes has been studied.

### Example 2

As used in this example and throughout the description the term FatTracer refers to a CRISPR loss-of-function screening platform as described above and the organoids generated using such methods. FatTracer may refer to wild type organoids (i.e. not including any of the modifications described herein) or to organoids including one or more of the modifications described herein.

### FatTracer Identifies FADS2 as a Critical Steatosis Determinant

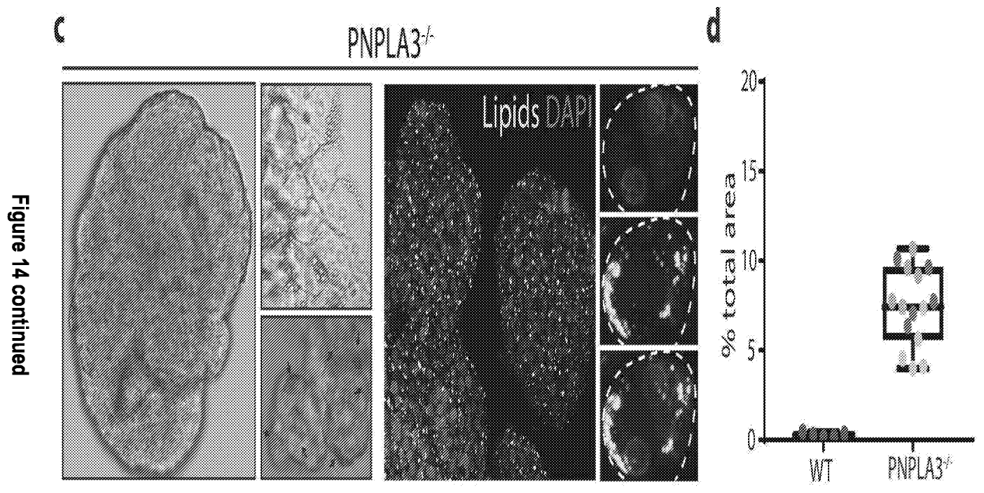

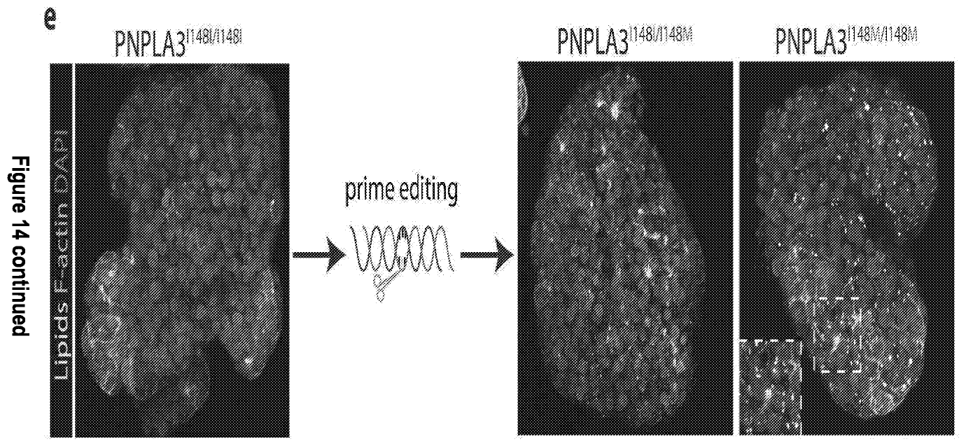

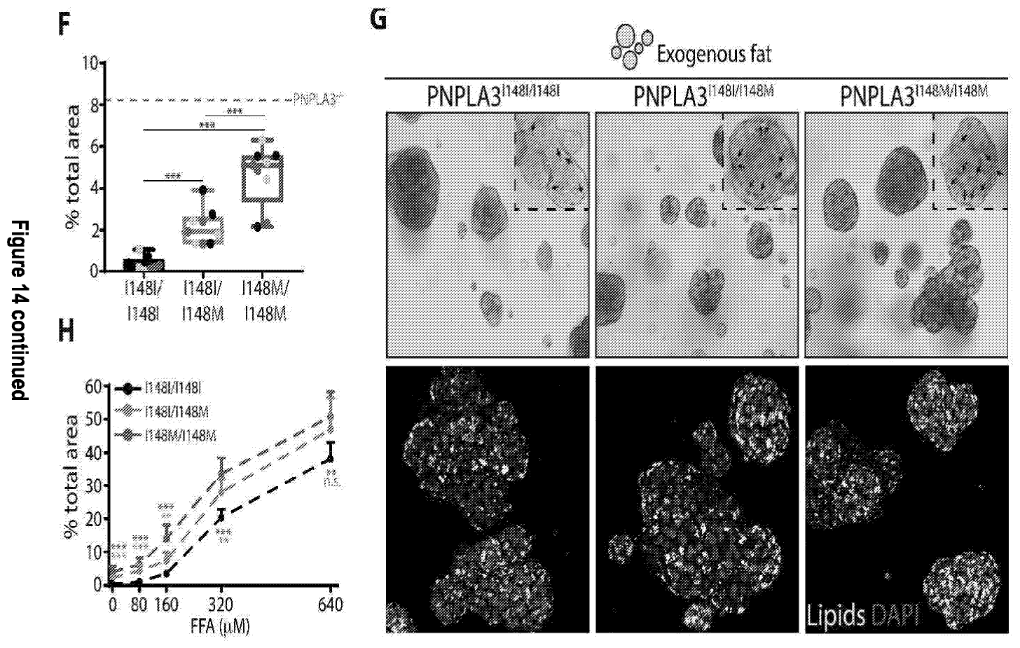

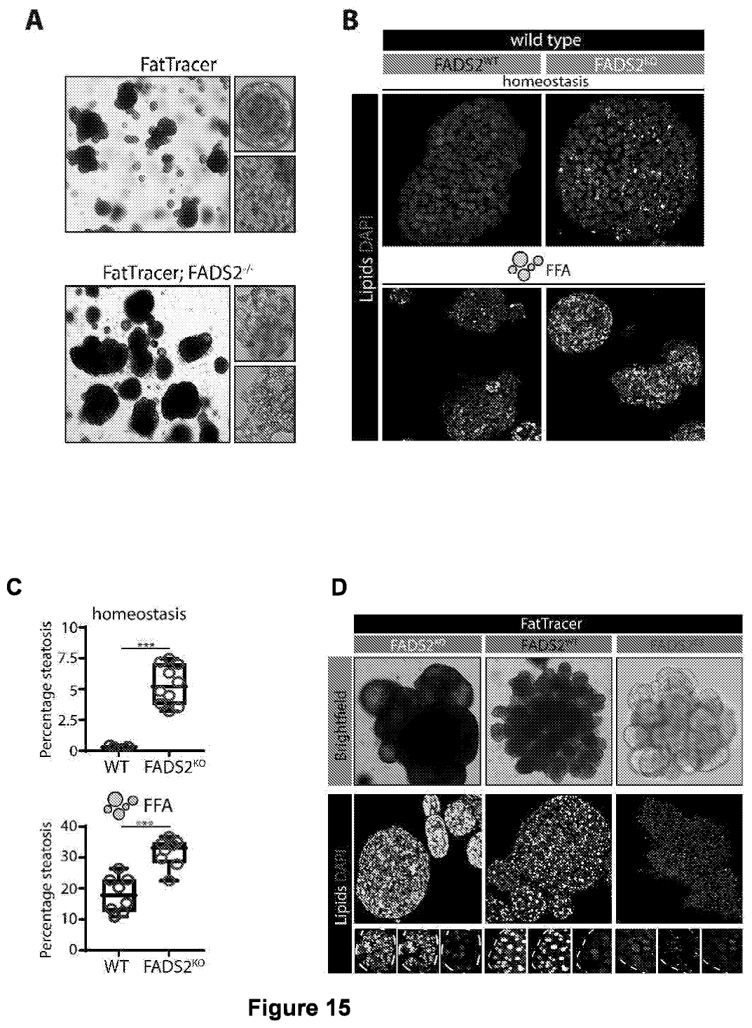

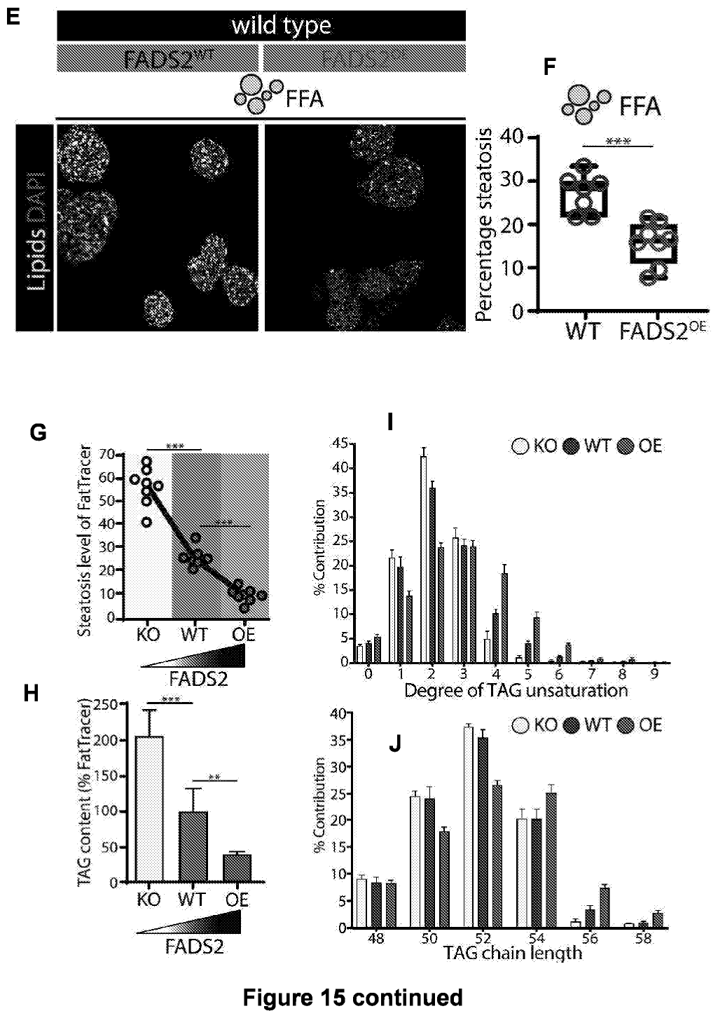

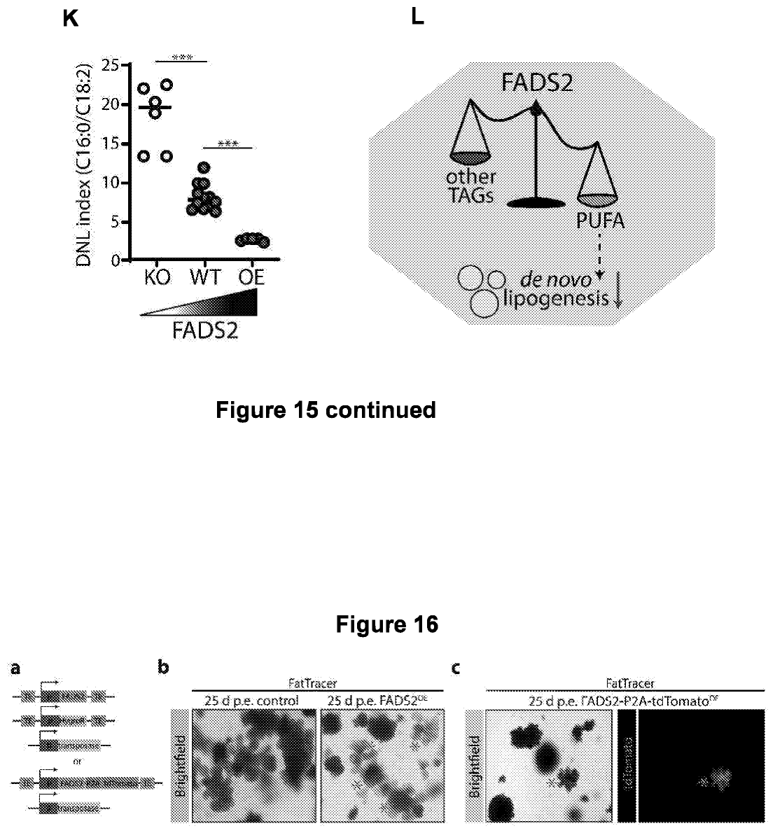

From the CRISPR screen described above, FADS2 emerged as a critical player in steatosis (FIGS. 11d, 11e, and 11g). FADS2 (fatty acid desaturase 2) is a delta-6 desaturase mediating the rate-limiting step in the biosynthesis of polyunsaturated fatty acids (PUFAs). A causative role of FADS2 in NAFLD has not previously been implied, nor has FADS2 been investigated as a potential target. Clonal FatTracer; FADS2^{-/-} lines presented as much darker (more lipid containing) organoids compared to FatTracer (FIG. 15A). To further evaluate the role of FADS2, it was questioned whether FADS2 knock-out would predispose to steatosis in wild type human hepatocytes. Therefore FADS2^{-/-} organoid lines were generated which indeed spontaneously accumulated lipids in basal culture conditions (ca. 5% steatosis) (FIG. 15B-C). These lines were then used to elucidate the interplay between the lack of FADS2 and a dietary steatosis trigger. When challenged with a mild dose of FFAs (320 mM), FADS2^{-/-} organoids displayed a marked aggravation of dietary steatosis (FIG. 15B-C), further strengthening the evidence of an important role for FADS2 in balancing lipid homeostasis.

FADS2 Overexpression Protects from Steatosis

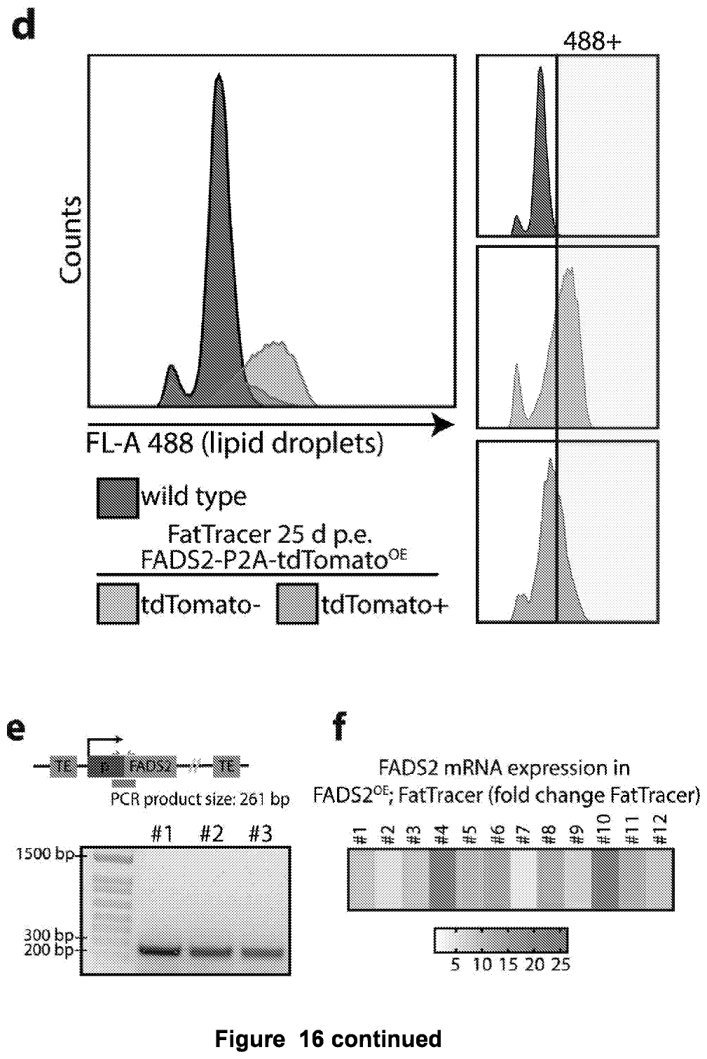

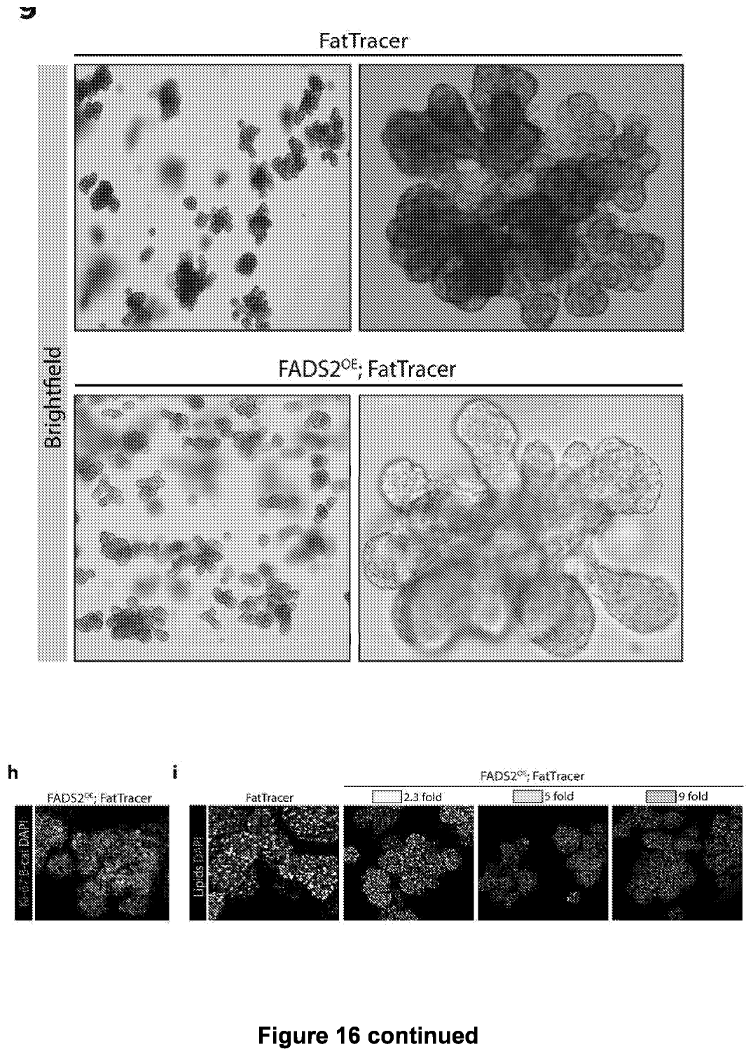

Prompted by these findings, it was investigated whether overexpression of FADS2 would be beneficial for the steatosis phenotype, both in terms of 1) resolving existing steatosis and/or 2) being protective against its development. A transposase-based integration of a CAG-FADS2 cassette to drive stable and constitutive overexpression of FADS2 (FADS2^{OE}) in transfected cells (FIG. 16A). First, FADS2 was overexpressed in FatTracer. Strikingly, the appearance of lighter-less lipid containing outgrowing organoids was observed (FIG. 16B-C). These findings were also corroborated by FACS analysis of the outgrowing organoids with a fluorescent lipid dye (FIG. 16D). Multiple clonal FatTracer; FADS2^{OE} lines (ranging from ca. 2-25-fold overexpression were derived (FIGS. 16E-F)) and it was found that FADS2 overexpression led to almost complete resolution of steatosis (ca. 70% reduction) (FIG. 15B, 15E, FIG. 16G), without altering the organoids' growth capacity and proliferation (FIG. 16H). Of note, only a mild threshold level of FADS2^{OE} was needed to resolve steatosis (FIG. 16I). Next generated FADS2^{OE} lines were generated in wild type organoids to evaluate whether FADS2 overexpression could confer protection against dietary steatosis. The same dosage of FFAs as used for the prior drug screenings was applied (500 mM) and it was found also that FADS2^{OE} markedly reduced the extent of lipid accumulation (FIG. 15E-F). Therefore, modulating the level of FADS2 expression directly determines the level of steatosis within hepatocytes (FIG. 15D, 15G), both in developing and pre-existing steatosis.

### Mechanisms of FADS2 Protection in Steatosis

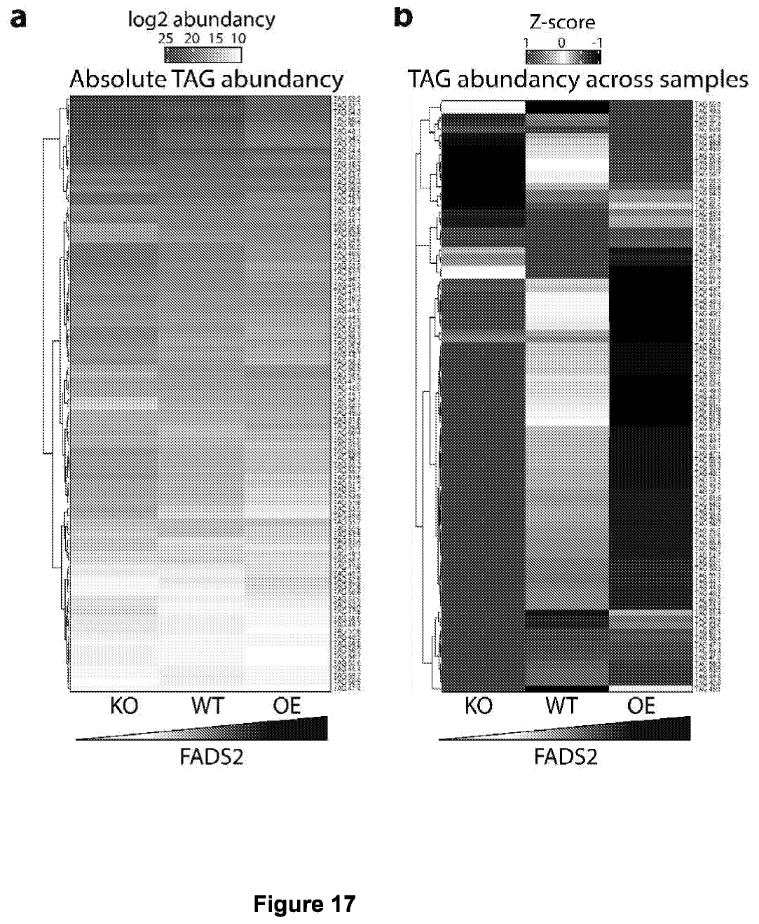

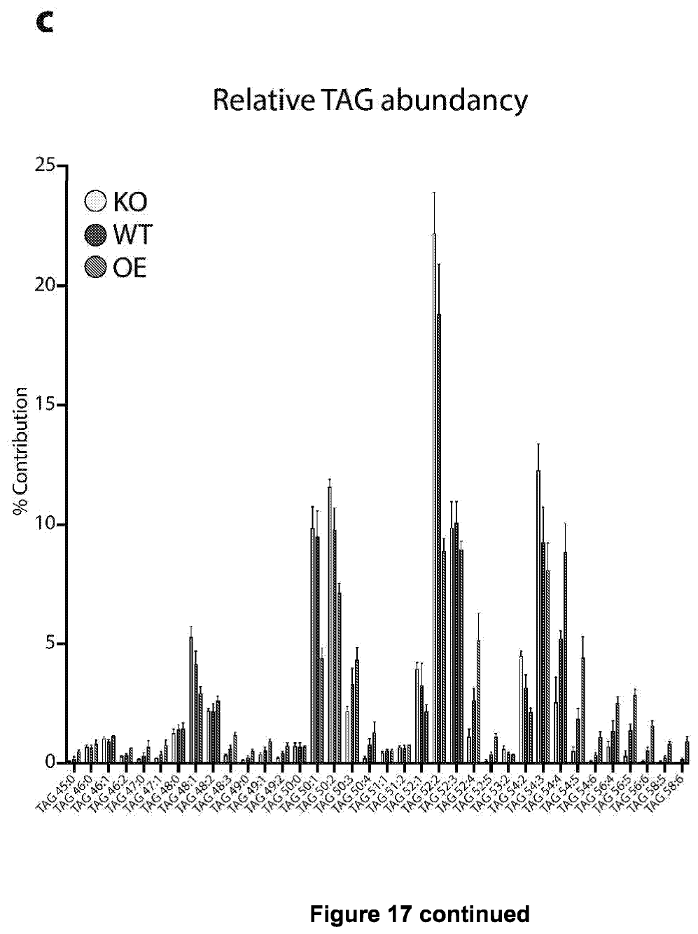

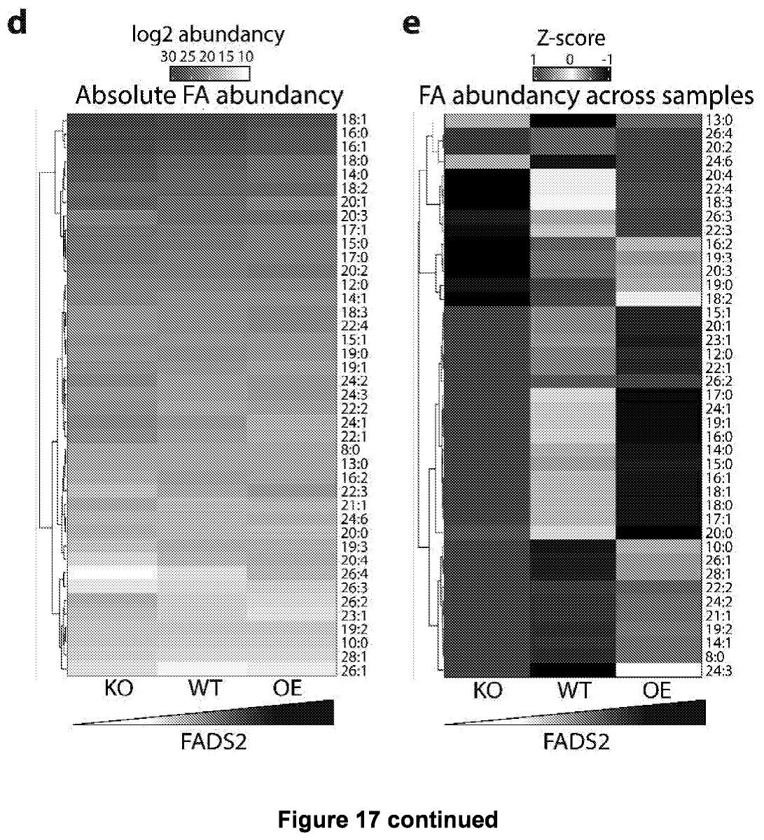

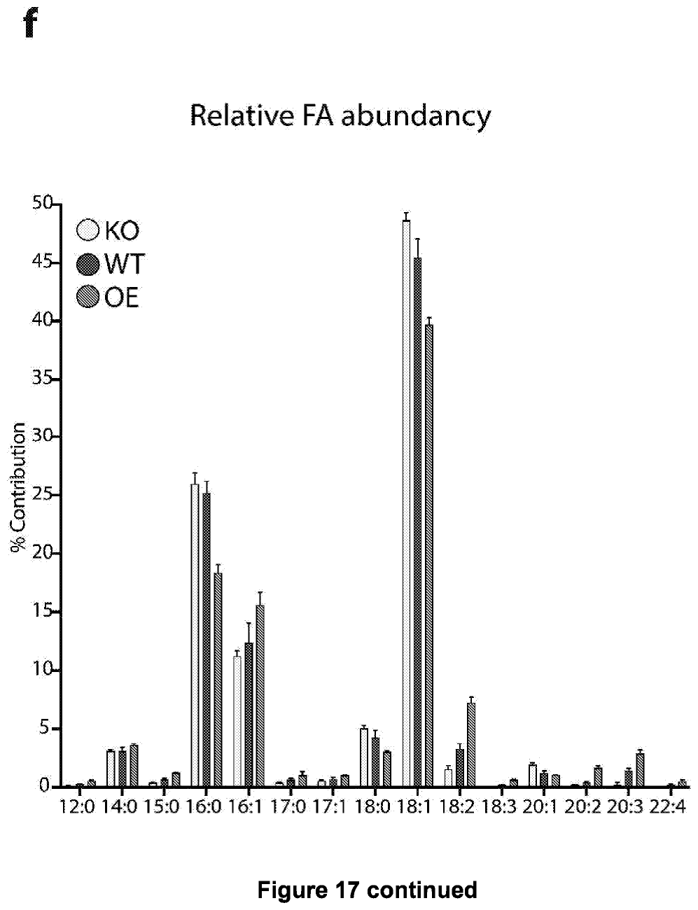

Given the role of FADS2 in fatty acid synthesis, the mechanistic cues we investigated by lipidomic analysis of FatTracer organoids in baseline (FADS2^{WT}) and upon FADS2^{-/-} and FADS2^{OE}. The total TAG content within these different FADS2 variant organoids was analyzed, which photocopied the observed steatosis trend. Relative to FatTracer, FADS2^{-/-} mutants had a ca. 200% increase in TAG content, while instead FADS2^{OE} organoids had a marked reduction of over 50% (FIG. 15F). The vast majority of TAG species were reduced upon FADS2^{OE} and conversely increased in FADS2^{-/-} (FIG. 17A-B). TAG and fatty acid complexity was then investigated (FIG. 17A-F). In line with FADS2 desaturase activity, TAG saturation was notably changed. FatTracer; FADS2^{OE} organoids were enriched with TAG with a higher degree of unsaturation, while conversely FADS2^{-/-} organoids displayed an increase in TAG with lower unsaturation levels (i.e. containing at least one saturated fatty acid) (FIG. 151).

TAG chain length analysis revealed a different trend. FADS2^{-/-} organoids displayed identical chain length profiles as compared to FatTracer, indicating that loss of FADS2 does not impair the overall generation of longer TAG species (FIG. 15J). The aggravated steatosis under loss of FADS2 is therefore solely caused by the increased abundancy in TAG species with lower degrees of unsaturation. Instead, FADS2^{OE} organoids were enriched for longer-chain TAG species (54C, 56C, 58C) at the expense of shorter TAG species (50C, 52C) (FIG. 15J). Shorter-chain TAG species represent newly synthesized fatty acids⁴⁶. A shift towards longer-chain “old” polyunsaturated fatty acids (PUFAs) upon FADS2 overexpression therefore suggested a decreased endogenous fatty acid production. To further test this hypothesis, the de novo lipogenesis (DNL) index (C16:0/C18:2)⁴⁷ in FADS2^{-/-}, FADS2^{WT}, FADS2^{OE} FatTracer organoids was calculated. The DNL index was sharply increased in FADS2^{-/-} while it was decreased in FADS2^{OE} organoids as compared to FADS2^{WT} organoids (FIG. 15K). Thus, a FADS2-regulated PUFA-DNL axis may represent a functional mechanism to influence steatosis levels within the hepatocyte (FIG. 15L).

### Example 3

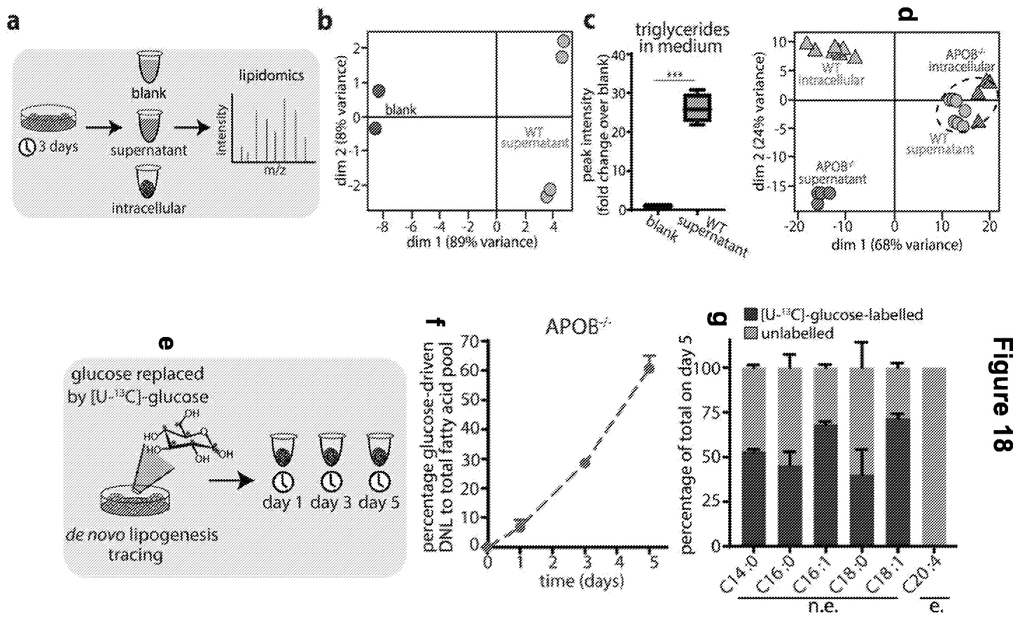

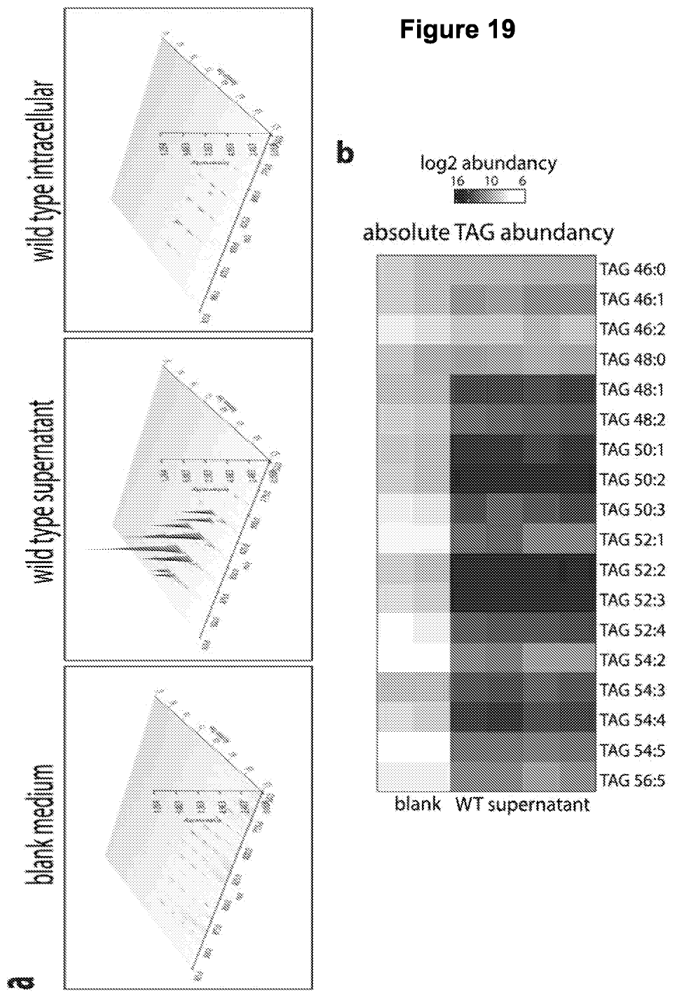

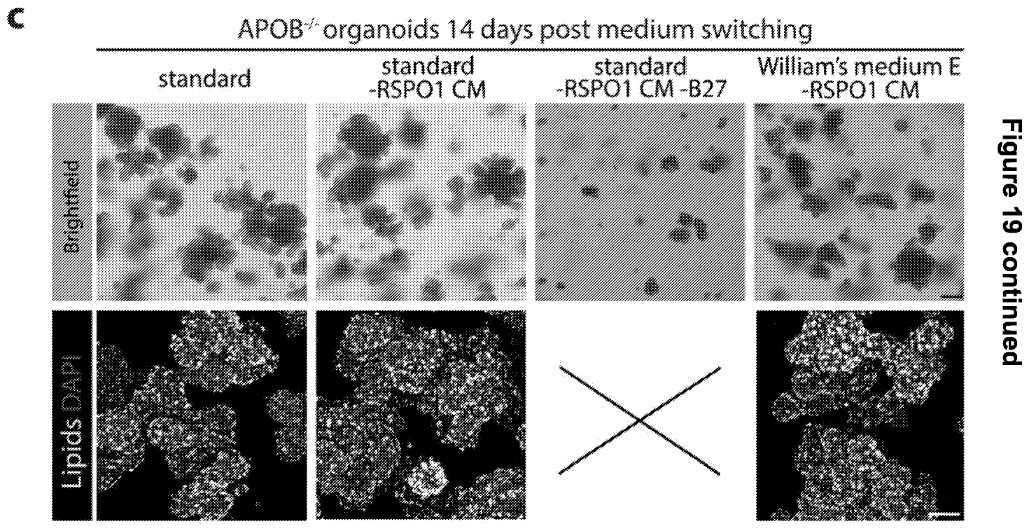

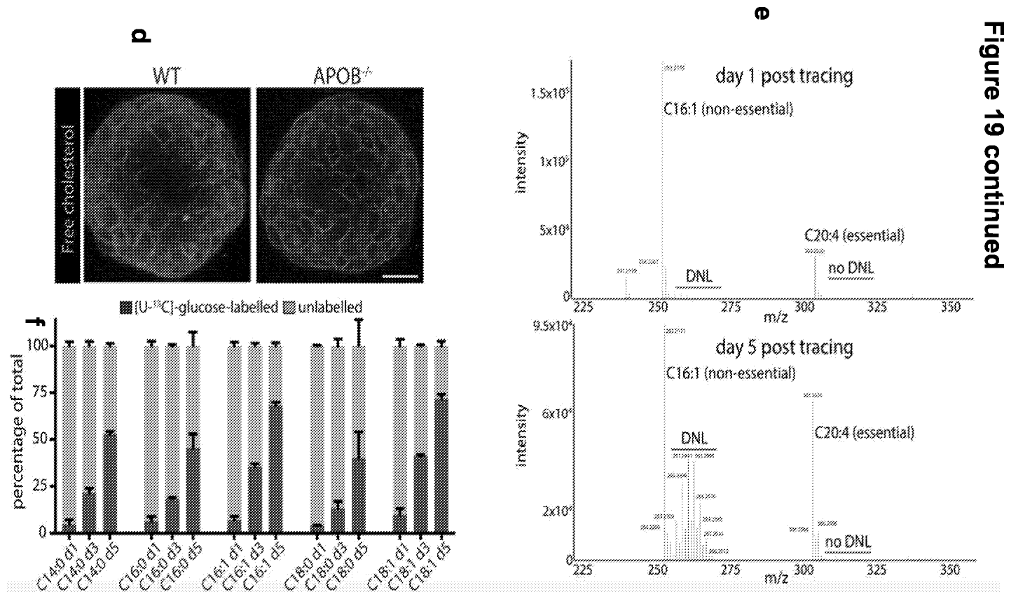

The VLDL secretory capacity of wild type organoids was addressed. To this end, organoids were cultured for 3 days without changing the culture medium, the supernatant collected, and lipidomics on neutral lipids was performed (FIG. 18a). Principle component analysis (PCA) revealed a distinct clustering of the lipid profiles of blank medium (which contained very few lipid species (FIG. 19a)) versus the medium in which wild type organoids were maintained (FIG. 18b). Indeed, TAG content in the medium was enriched by ca. 25-fold (FIG. 18c, FIG. 19a-b), confirming that wild type organoids actively secrete VLDL particles.

It was reasoned that de novo lipogenesis (DNL) should be the source of lipid accumulation in APOB^{-/-} and MTTP^{-/-} organoids, since the culture medium itself contains minimal lipid sources and only small amounts of essential fatty acids (i.e. those that cells cannot synthesize). Additionally, culturing these mutants in absence of the potential confounding lipid sources (e.g. RSPO1-conditioned medium), did not alter the steatosis phenotypes (FIG. 19c). To directly probe the origin and to assess changes in lipid profiles, both APOB^{-/-} and wild type organoids were subjected to lipidomic analyses, this time interrogating both the intracellular lipid profiles (organoid pellets) and the secreted lipid profiles (supernatants) (FIG. 18a).

Strikingly, PCA revealed distinct clustering, where the intracellular lipid profiles of APOB^{-/-} organoids closely clustered with the supernatants of wild type organoids, confirming defective lipid export in ApoB mutants (FIG. 18d). Quantification of the lipid content corroborated their extensive accumulation within APOB^{-/-} organoids (ca. 14-fold compared to wild type), with the predominant change being extensive TAG accumulation, while conversely lipids (especially TAG) in the supernatant were near absent. To address the contribution of DNL to the observed lipid profiles, a time-resolved [U-¹³C]-glucose isotope tracing experiment was performed (FIG. 18e). The incorporation of labelled glucose into the synthesis of 5 of the most abundant fatty acids (C14:0, C16:0, C16:1, C18:0, and C18:1) was calculated which together constitute>90% of the total intracellular fatty acid pool and >85% of the fatty acid pool that can be synthesized by DNL. A time-dependent increase in the glucose-dependent DNL contribution was observed, which linearly increased to ca. 60% on day 5, with a low extent of heterogeneity between the contribution of the individual fatty acids (FIG. 18f-g). The validity of this approach was confirmed by the absence of labeling of essential fatty acids (e.g. C20:4) (FIG. 18g, FIG. 19d). A 100% incorporation would not be expected, as other sources contributing to the acetyl-CoA pool, such as amino acids and culture medium-provided pyruvate and glutamine, were not traced in this setting. Thus, glucose-driven DNL is the main contributor to the spontaneous steatosis phenotype of these lipid defective organoids. Both APOB^{-/-} and MTTP^{-/-} mutants constitute natural steatosis organoid models that can, like their wild type counterparts, be long-term expanded in culture for at least 2 years with stable steatosis levels.

### REFERENCES

Anzalone, A. v., Randolph, P. B., Davis, J. R., Sousa, A. A., Koblan, L. W., Levy, J. M., Chen, P. J., Wilson, C., Newby, G. A., Raguram, A., et al. (2019). Search-and-replace genome editing without double-strand breaks or donor DNA. Nature 576.
Artegiani, B., and Clevers, H. (2018). Use and application of 3D-organoid technology. Human Molecular Genetics 27.
Artegiani, B., Hendriks, D., Beumer, J., Kok, R., Zheng, X., Joore, I., Chuva de Sousa Lopes, S., van Zon, J., Tans, S., and Clevers, H. (2020). Fast and efficient generation of knock-in human organoids using homology-independent CRISPR-Cas9 precision genome editing. Nature Cell Biology 22.
Basantani, M. K., Sitnick, M. T., Cai, L., Brenner, D. S., Gardner, N. P., Li, J. Z., Schoiswohl, G., Yang, K., Kumari, M., Gross, R. W., et al. (2011). Pnpla3/Adiponutrin deficiency in mice does not contribute to fatty liver disease or metabolic syndrome. Journal of Lipid Research 52.
BasuRay, S., Wang, Y., Smagris, E., Cohen, J. C., and Hobbs, H. H. (2019). Accumulation of PNPLA3 on lipid droplets is the basis of associated hepatic steatosis. Proceedings of the National Academy of Sciences 116.
Bligh, E. G., and Dyer, W. J. (1959). A rapid method of total lipid extraction and purification. Canadian Journal of Biochemistry and Physiology 37.
Calkin, A. C., and Tontonoz, P. (2012). Transcriptional integration of metabolism by the nuclear sterol-activated receptors LXR and FXR. Nature Reviews Molecular Cell Biology 13.
Cao, W., Collins, Q. F., Becker, T. C., Robidoux, J., Lupo, E. G., Xiong, Y., Daniel, K. W., Floering, L., and Collins, S. (2005). p38 Mitogen-activated Protein Kinase Plays a Stimulatory Role in Hepatic Gluconeogenesis. Journal of Biological Chemistry 280.
Caunt, C. J., and Keyse, S. M. (2013). Dual-specificity MAP kinase phosphatases (MKPs). The FEBS Journal 280.
Cefalù, A. B., Pirruccello, J. P., Noto, D., Gabriel, S., Valenti, V., Gupta, N., Spina, R., Tarugi, P., Kathiresan, S., and Averna, M. R. (2013). A Novel APOB Mutation Identified by Exome Sequencing Cosegregates With Steatosis, Liver Cancer, and Hypocholesterolemia. Arteriosclerosis, Thrombosis, and Vascular Biology 33.
Chakravarthy, M. v., Lodhi, I. J., Yin, L., Malapaka, R. R. V., Xu, H. E., Turk, J., and Semenkovich, C. F. (2009). Identification of a Physiologically Relevant Endogenous Ligand for PPARα in Liver. Cell 138.
Chambers, J. C., Zhang, W., Sehmi, J., Li, X., Wass, M. N., van der Harst, P., Holm, H., Sanna, S., Kavousi, M., Baumeister, S. E., et al. (2011). Genome-wide association study identifies loci influencing concentrations of liver enzymes in plasma. Nature Genetics 43.
Chen, W., Chang, B., Li, L., and Chan, L. (2010). Patatin-like phospholipase domain-containing 3/adiponutrin deficiency in mice is not associated with fatty liver disease. Hepatology 52.
Chow, R. D., Chen, J. S., Shen, J., and Chen, S. (2021). A web tool for the design of prime-editing guide RNAs. Nature Biomedical Engineering 5.
Collin de l'Hortet, A., Takeishi, K., Guzman-Lepe, J., Morita, K., Achreja, A., Popovic, B., Wang, Y., Handa, K., Mittal, A., Meurs, N., et al. (2019). Generation of Human Fatty Livers Using Custom-Engineered Induced Pluripotent Stem Cells with Modifiable SIRT1 Metabolism. Cell Metabolism 30.
Dong, X. C. (2019). PNPLA3—A Potential Therapeutic Target for Personalized Treatment of Chronic Liver Disease. Frontiers in Medicine 6.
Esler, W. P., and Bence, K. K. (2019). Metabolic Targets in Nonalcoholic Fatty Liver Disease. Cellular and Molecular Gastroenterology and Hepatology 8.
Foti, D., Chiefari, E., Fedele, M., Iuliano, R., Brunetti, L., Paonessa, F., Manfioletti, G., Barbetti, F., Brunetti, A., Croce, C. M., et al. (2005). Lack of the architectural factor HMGA1 causes insulin resistance and diabetes in humans and mice. Nature Medicine 11.
Hendriks, D., Clevers, H., and Artegiani, B. (2020). CRISPR-Cas Tools and Their Application in Genetic Engineering of Human Stem Cells and Organoids. Cell Stem Cell 27.
Hendriks, D., Artegiani, B., Hu, H., Chuva de Sousa Lopes, S., and Clevers, H. (2021). Establishment of human fetal hepatocyte organoids and CRISPR-Cas9-based gene knockin and knockout in organoid cultures from human liver. Nature Protocols 16.
van Herck, M., Vonghia, L., and Francque, S. (2017). Animal Models of Nonalcoholic Fatty Liver Disease—A Starter's Guide. Nutrients 9.
Hu, H., Gehart, H., Artegiani, B., LÖpez-Iglesias, C., Dekkers, F., Basak, O., van Es, J., Chuva de Sousa Lopes, S. M., Begthel, H., Korving, J., et al. (2018). Long-Term Expansion of Functional Mouse and Human Hepatocytes as 3D Organoids. Cell 175.
Itabe, H., Yamaguchi, T., Nimura, S., and Sasabe, N. (2017). Perilipins: a diversity of intracellular lipid droplet proteins. Lipids in Health and Disease 16.
Jeucken, A., Molenaar, M. R., van de Lest, C. H. A., Jansen, J. W. A., Helms, J. B., and Brouwers, J. F. (2019). A Comprehensive Functional Characterization of Escherichia coli Lipid Genes. Cell Reports 27.
Karavia, E. A., Papachristou, D. J., Liopeta, K., Triantaphyllidou, I.-E., Dimitrakopoulos, O., and Kypreos, K. E. (2012). Apolipoprotein A-I Modulates Processes Associated with Diet-Induced Nonalcoholic Fatty Liver Disease in Mice. Molecular Medicine 18.
Kim, C.-W., Addy, C., Kusunoki, J., Anderson, N. N., Deja, S., Fu, X., Burgess, S. C., Li, C., Ruddy, M., Chakravarthy, M., et al. (2017). Acetyl CoA Carboxylase Inhibition Reduces Hepatic Steatosis but Elevates Plasma Triglycerides in Mice and Humans: A Bedside to Bench Investigation. Cell Metabolism 26.
Kleiner, D. E., Brunt, E. M., van Natta, M., Behling, C., Contos, M. J., Cummings, O. W., Ferrell, L. D., Liu, Y.-C., Torbenson, M. S., Unalp-Arida, A., et al. (2005). Design and validation of a histological scoring system for nonalcoholic fatty liver disease. Hepatology 41.
Kozlitina, J., Smagris, E., Stender, S., Nordestgaard, B. G., Zhou, H. H., Tybjærg-Hansen, A., Vogt, T. F., Hobbs, H. H., and Cohen, J. C. (2014). Exome-wide association study identifies a TM6SF2 variant that confers susceptibility to nonalcoholic fatty liver disease. Nature Genetics 46.
Kozyra, M., Johansson, I., Nordling, Å., Ullah, S., Lauschke, V. M., and Ingelman-Sundberg, M. (2018). Human hepatic 3D spheroids as a model for steatosis and insulin resistance. Scientific Reports 8.
Lefere, S., Puengel, T., Hundertmark, J., Penners, C., Frank, A. K., Guillot, A., de Muynck, K., Heymann, F., Adarbes, V., Defrêne, E., et al. (2020). Differential effects of selective- and pan-PPAR agonists on experimental steatohepatitis and hepatic macrophages⋆. Journal of Hepatology 73.
Li, J. Z., Huang, Y., Karaman, R., Ivanova, P. T., Brown, H. A., Roddy, T., Castro-Perez, J., Cohen, J. C., and Hobbs, H. H. (2012). Chronic overexpression of PNPLA31148M in mouse liver causes hepatic steatosis. Journal of Clinical Investigation 122.
Liebe, R., Esposito, I., Bock, H. H., vom Dahl, S., Stindt, J., Baumann, U., Luedde, T., and Keitel, V. (2021). Diagnosis and management of secondary causes of steatohepatitis. Journal of Hepatology 74.
Lindén, D., Ahnmark, A., Pingitore, P., Ciociola, E., Ahlstedt, I., Andréasson, A.-C., Sasidharan, K., Madeyski-Bengtson, K., Zurek, M., Mancina, R. M., et al. (2019). Pnpla3 silencing with antisense oligonucleotides ameliorates nonalcoholic steatohepatitis and fibrosis in Pnpla3 I148M knock-in mice. Molecular Metabolism 22.
Loomba, R., Friedman, S. L., and Shulman, G. I. (2021). Mechanisms and disease consequences of nonalcoholic fatty liver disease. Cell 184.
Love, M. I., Huber, W., and Anders, S. (2014). Moderated estimation of fold change and dispersion for RNA-seq data with DESeq2. Genome Biology 15.
Luu, W., Sharpe, L. J., Capell-Hattam, I., Gelissen, I. C., and Brown, A. J. (2016). Oxysterols: Old Tale, New Twists. Annual Review of Pharmacology and Toxicology 56.
Luukkonen, P. K., Nick, A., Hölttä-Vuori, M., Thiele, C., Isokuortti, E., Lallukka-Brück, S., Zhou, Y., Hakkarainen, A., Lundbom, N., Peltonen, M., et al. (2019). Human PNPLA3-I148M variant increases hepatic retention of polyunsaturated fatty acids. JCI Insight 4.
Mancina, R. M., Dongiovanni, P., Petta, S., Pingitore, P., Meroni, M., Rametta, R., Borén, J., Montalcini, T., Pujia, A., Wiklund, O., et al. (2016). The MBOAT7-TMC4 Variant rs641738 Increases Risk of Nonalcoholic Fatty Liver Disease in Individuals of European Descent. Gastroenterology 150.
Mullard, A. (2020). FDA rejects NASH drug. Nature Reviews Drug Discovery 19.
Ouchi, R., Togo, S., Kimura, M., Shinozawa, T., Koido, M., Koike, H., Thompson, W., Karns, R. A., Mayhew, C. N., McGrath, P. S., et al. (2019). Modeling Steatohepatitis in Humans with Pluripotent Stem Cell-Derived Organoids. Cell Metabolism 30.
Pirazzi, C., Adiels, M., Burza, M. A., Mancina, R. M., Levin, M., Ståhlman, M., Taskinen, M.-R., Orho-Melander, M., Perman, J., Pujia, A., et al. (2012). Patatin-like phospholipase domain-containing 3 (PNPLA3) I148M (rs738409) affects hepatic VLDL secretion in humans and in vitro. Journal of Hepatology 57.
Qi, J., Lang, W., Geisler, J. G., Wang, P., Petrounia, I., Mai, S., Smith, C., Askari, H., Struble, G. T., Williams, R., et al. (2012). The use of stable isotope-labeled glycerol and oleic acid to differentiate the hepatic functions of DGAT1 and 2. Journal of Lipid Research 53.
Ramli, M. N. bin, Lim, Y. S., Koe, C. T., Demircioglu, D., Tng, W., Gonzales, K. A. U., Tan, C. P., Szczerbinska, I., Liang, H., Soe, E. L., et al. (2020). Human Pluripotent Stem Cell-Derived Organoids as Models of Liver Disease. Gastroenterology 159.
Ran, F. A., Hsu, P. D., Wright, J., Agarwala, V., Scott, D. A., and Zhang, F. (2013). Genome engineering using the CRISPR-Cas9 system. Nature Protocols 8.
Romeo, S., Kozlitina, J., Xing, C., Pertsemlidis, A., Cox, D., Pennacchio, L. A., Boerwinkle, E., Cohen, J. C., and Hobbs, H. H. (2008). Genetic variation in PNPLA3 confers susceptibility to nonalcoholic fatty liver disease. Nature Genetics 40.
Schonfeld, G., Lin, X., and Yue, P. (2003a). Familial hypobetalipoproteinemia: a review. Journal of Lipid Research 44.
Schonfeld, G., Patterson, B. W., Yablonskiy, D. A., Tanoli, T. S. K., Averna, M., Elias, N., Yue, P., and Ackerman, J. (2003b). Fatty liver in familial hypobetalipoproteinemia: triglyceride assembly into VLDL particles is affected by the extent of hepatic steatosis. Journal of Lipid Research 44.
Schutgens, F., and Clevers, H. (2020). Human Organoids: Tools for Understanding Biology and Treating Diseases. Annual Review of Pathology: Mechanisms of Disease 15.
Smagris, E., BasuRay, S., Li, J., Huang, Y., Lai, K. v., Gromada, J., Cohen, J. C., and Hobbs, H. H. (2015). Pnpla3I148M knockin mice accumulate PNPLA3 on lipid droplets and develop hepatic steatosis. Hepatology 61.
Smith, C. A., Want, E. J., O'Maille, G., Abagyan, R., and Siuzdak, G. (2006). XCMS: Processing Mass Spectrometry Data for Metabolite Profiling Using Nonlinear Peak Alignment, Matching, and Identification. Analytical Chemistry 78.
Sołtysik, K., Ohsaki, Y., Tatematsu, T., Cheng, J., and Fujimoto, T. (2019). Nuclear lipid droplets derive from a lipoprotein precursor and regulate phosphatidylcholine synthesis. Nature Communications 10.
Song, K.-H., Li, T., Owsley, E., Strom, S., and Chiang, J. Y. L. (2009). Bile acids activate fibroblast growth factor 19 signaling in human hepatocytes to inhibit cholesterol 7α-hydroxylase gene expression. Hepatology 49.
Speliotes, E. K., Yerges-Armstrong, L. M., Wu, J., Hernaez, R., Kim, L. J., Palmer, C. D., Gudnason, V., Eiriksdottir, G., Garcia, M. E., Launer, L. J., et al. (2011). Genome-Wide Association Analysis Identifies Variants Associated with Nonalcoholic Fatty Liver Disease That Have Distinct Effects on Metabolic Traits. PLOS Genetics 7.
Stacklies, W., Redestig, H., Scholz, M., Walther, D., and Selbig, J. (2007). pcaMethods a bioconductor package providing PCA methods for incomplete data. Bioinformatics 23.
Stefan, N., Häring, H.-U., and Cusi, K. (2019). Non-alcoholic fatty liver disease: causes, diagnosis, cardiometabolic consequences, and treatment strategies. The Lancet Diabetes & Endocrinology 7.
Tilson, S. G., Morell, C. M., Lenaerts, A., Park, S. B., Hu, Z., Jenkins, B., Koulman, A., Liang, T. J., and Vallier, L. (2021). Modelling PNPLA3-Associated Non-Alcoholic Fatty Liver Disease Using Human Induced Pluripotent Stem Cells. Hepatology.
Trépo, E., and Valenti, L. (2020). Update on NAFLD genetics: From new variants to the clinic. Journal of Hepatology 72.
Wang, B., and Tontonoz, P. (2018). Liver X receptors in lipid signalling and membrane homeostasis. Nature Reviews Endocrinology 14.
Welty, F. K. (2014). Hypobetalipoproteinemia and abetalipoproteinemia. Current Opinion in Lipidology 25.
Wetterau, Aggerbeck, L., Bouma, M., Eisenberg, C., Munck, A., Hermier, M., Schmitz, J., Gay, G., Rader, D., and Gregg, R. (1992). Absence of microsomal triglyceride transfer protein in individuals with abetalipoproteinemia. Science 258.
Younossi, Z., Tacke, F., Arrese, M., Chander Sharma, B., Mostafa, I., Bugianesi, E., Wai-Sun Wong, V., Yilmaz, Y., George, J., Fan, J., et al. (2019). Global Perspectives on Nonalcoholic Fatty Liver Disease and Nonalcoholic Steatohepatitis. Hepatology 69.
Younossi, Z. M., Koenig, A. B., Abdelatif, D., Fazel, Y., Henry, L., and Wymer, M. (2016). Global epidemiology of nonalcoholic fatty liver disease-Meta-analytic assessment of prevalence, incidence, and outcomes. Hepatology 64.
Zeilinger, K., Freyer, N., Damm, G., Seehofer, D., and Knöspel, F. (2016). Cell sources for in vitro human liver cell culture models. Experimental Biology and Medicine 241.
Zhang, X., Zhang, P., Gao, J., and Huang, Q. (2018). Autophagy dysregulation caused by ApoM deficiency plays an important role in liver lipid metabolic disorder. Biochemical and Biophysical Research Communications 495.

### CLAUSES

1. A human hepatocyte organoid comprising at least one of:
a modified Apolipoprotein B-100 (APOB) gene;
a modified Microsomal Triglyceride Transfer Protein (MTTP) gene;
a modified FADS2 gene; and/or
a modified PNPLA3 gene.
   2. The human hepatocyte organoid of clause 1, comprising:
a) a modified Apolipoprotein B-100 (APOB) gene;
b) a modified Microsomal Triglyceride Transfer Protein (MTTP) gene;
c) a modified FADS2 gene;
d) a modified PNPLA3 gene;
e) a modified Apolipoprotein B-100 (APOB) gene and a modified FADS2 gene;
f) a modified Apolipoprotein B-100 (APOB) gene and a modified PNPLA3 gene;
g) a modified Microsomal Triglyceride Transfer Protein (MTTP) gene and a modified FADS2 gene; or
h) a modified Microsomal Triglyceride Transfer Protein (MTTP) gene and a modified PNPLA3 gene.
   3. The human hepatocyte organoid of clauses 1 or 2, wherein the modification comprises a mutation or deletion; optionally wherein at least one of the modified Apolipoprotein B-100 (APOB) gene, the modified Microsomal Triglyceride Transfer Protein (MTTP) gene, the modified FADS2 gene, and/or the modified PNPLA3 gene are attenuated.
   4. The human hepatocyte organoid of any of clauses 1 to 3, wherein the human hepatocyte organoid comprise lipids droplets, wherein the lipid droplets occupy a greater area of the human hepatocyte organoid in comparison to a wild type human hepatocyte organoid.
   5. The human hepatocyte organoid of any preceding clause, wherein human hepatocyte organoid comprises altered lipid homeostasis.
   6. The human hepatocyte organoid of any preceding clause, wherein the human hepatocyte organoid accumulates lipids via de novo lipogenesis-driven steatosis; optionally wherein the human hepatocyte organoid is according to any of clause 2 a), b), e), f), g), or h).
   7. The human hepatocyte organoid of any preceding clause, wherein the human hepatocyte organoid is a tissue derived human hepatocyte organoid.
   8. The human hepatocyte organoid of any preceding clause, wherein the human hepatocyte organoid further comprises exogenous lipids.
   9. The human hepatocyte organoid of clause 2 a), b), e), f), g), or h) comprising downregulation of at least one LXR-regulated gene in comparison to a wild type human hepatocyte organoid.
   10. The human hepatocyte organoid of clause 9, wherein the at least one LXR-regulated gene comprises one or more of ACACA, FASN, DGAT2, SREBF1, HMGCS1, SQLE, LSS, and/or DHCR7.
   11. A method of forming a human hepatocyte organoid for modelling lipid homeostasis, the method comprising:
a. providing a human hepatocyte organoid;
b. modifying at least one of:
   i. at least one Apolipoprotein B-100 (APOB) gene; or
   ii. at least one Microsomal Triglyceride Transfer Protein (MTTP) gene;
   iii. at least one FADS2 gene; and/or
   iv. at least one PNPLA3 gene;
c. recovering cells comprising the modified APOB, MTTP, FADS2, and/or PNPLA3 genes; and
d. culturing the cells to form human hepatocyte organoids.
   12. The method of clause 11, wherein modifying comprises CRISPR based gene disruption.
   13. The method of clause 12, wherein CRISPR based gene disruption comprises introducing into cells of the human hepatocyte organoid one or more vectors for disrupting the APOB, MTTP FADS2, and/or PNPLA3 genes, the at least one vector comprising at least one of a guide RNA for targeting APOB, MTTP FADS2, and/or PNPLA3 and/or a Cas9 enzyme.
   14. Use of a human hepatocyte organoid according to any of clauses 1 to 10 or formed by the methods of any of clauses 11 to 13 for modelling lipid homeostasis.
   15. The use of clause 14, wherein the human hepatocyte organoid is for modelling steatosis
   16. The use of clause 15, wherein the steatosis is de novo lipogenesis driven steatosis.
   17. The use of clauses 14 to 16, wherein the human hepatocyte organoid is for modelling NAFLD, NASH and/or liver cancer.
   18. A p38 inhibitor, FADS2 agonist, ACC inhibitor, DGAT2 inhibitor, FAS inhibitor, recombinant hFGF19 or FXR agonist for use in treating NAFLD in a subject in need thereof, wherein the subject comprises at least one of:
a. at least one modified PNPLA3 gene;
b. at least one modified FADS2 gene;
c. at least one modified APOB gene; and/or
d. at least one modified MTTP gene.
   19. The p38 inhibitor, FADS2 agonist, ACC inhibitor, FXR agonist, DGAT2 inhibitor, FAS inhibitor or hFGF19 for use according to clause 18, wherein the subject suffers from familial hypobetalipoproteinaemia (FHBL).
   20. The p38 inhibitor, FADS2 agonist, ACC inhibitor, FXR agonist, DGAT2 inhibitor, FAS inhibitor or hFGF19 for use according to clause 19, wherein the familial hypobetalipoproteinaemia is associated with the least one attenuating APOB mutation.
   21. The p38 inhibitor, FADS2 agonist, ACC inhibitor, FXR agonist, DGAT2 inhibitor, FAS inhibitor or hFGF19 for use according to any one of clauses 18 to 20, wherein the subject suffers from abetalipoproteinemia (ABL).
   22. The p38 inhibitor, FADS2 agonist, ACC inhibitor, FXR agonist, DGAT2 inhibitor, FAS inhibitor or hFGF19 for use according to clause 21, wherein the abetalipoproteinemia is associated with the at least one attenuating MTTP mutation.
   23. The p38 inhibitor, FADS2 agonist, ACC inhibitor, FXR agonist, DGAT2 inhibitor, FAS inhibitor or hFGF19 for use according to any one of clauses 18 to 22 wherein the at least one modified PNPLA3 comprises a homozygous or heterozygous PNPLA3 I148M mutation.
   24. The p38 inhibitor, FADS2 agonist, ACC inhibitor, FXR agonist, DGAT2 inhibitor, FAS inhibitor or hFGF19 for use according to any one of clauses 18 to 23, wherein the at least one modified FADS2 comprises a single nucleotide polymorphism.
   25. A method reducing steatosis in hepatocytes in a subject in need thereof, comprising administering an agent targeting de novo lipogenesis to the subject, wherein the subject comprises at least one of:
a. at least one modified PNPLA3 gene;
b. at least one modified FADS2 gene;
c. at least one modified APOB gene; and/or
d. at least one modified MTTP gene.
   26. The method according to clause 25, wherein the agent comprises at least one of:
p38 inhibitor;
FADS2 agonist;
ACC inhibitor;
FXR agonist;
FAS inhibitor;
DGAT2 inhibitor; and/or
recombinant hFGF19.
   27. The method according to any of clauses 25 or 26, wherein the subject suffers from NAFLD.
   28. A method of treating NAFLD comprising inducing Dual Specificity Phosphatase 4 and/or Dual Specificity Phosphatase 5 in a subject in need thereof.
   29 The method of clause 28, wherein inducing Dual Specificity Phosphatase 4 and/or Dual Specificity Phosphatase 5 comprises administering an agent that inhibits p38 signalling.
   30. A FADS2 agonist for use in treating or preventing NAFLD in a subject in need thereof.
   31. A FADS2 agonist for use in treating or preventing a cardiovascular disease in a subject in need thereof.
   32. A FADS2 agonist for use in reducing and/or preventing steatosis in a subject in need thereof.
   33. The FADS2 agonist according to clause 32, wherein the steatosis is dietary induced steatosis.
   34. A FADS2 agonist for use in preventing and/or reducing the risk of NASH and/or cirrhosis in a subject in need thereof.
   35. The FADS2 agonist for use according to any one of clauses 30 to 34, wherein the FADS2 agonist comprises an agent for increasing activity of FADS2 in the subject.
   36. The FADS2 agonist for use according to clauses 30 to 35, wherein the FADS2 agonist comprises an agent for increasing expression of endogenous and/or exogenous FADS2 in the subject.
   37. The FADS2 agonist for use according to clauses 35 or 36, wherein the agent comprises a nucleic acid that encodes a FADS2 agonist; optionally wherein the FADS2 agonist comprises a nucleic acid that encodes an endogenous and/or exogenous FADS2 polypeptide; optionally wherein the FADS2 polypeptide comprises a human FADS2 polypeptide.
   38. The FADS2 agonist for use according to claim 35 or 36, wherein the agent comprises a polypeptide encoding an exogenous and/or endogenous FADS2 polypeptide optionally wherein the FADS2 polypeptide comprises a human FADS2 polypeptide.
   39. The FADS2 agonist for use according to any one of clauses 30 to 37 wherein the FDS2 agonist comprises an expression vector, the expression vector comprising a nucleic acid or FADS2 polypeptide according to clauses 37 or 38.
   40. A method of treating or preventing a cardiovascular disease in a subject in need thereof, comprising increasing FADS2 activity in the subject.
   41. A method of reducing and/or preventing steatosis in a subject in need thereof, comprising increasing FADS2 activity in the subject.
   42. A method of treating or preventing NAFLD in a subject in need thereof, comprising increasing FADS2 activity in the subject.
   43. A method of preventing and/or reducing the risk of NASH and/or cirrhosis in a subject in need thereof, comprising increasing FADS2 activity in the subject.
   44. The method according to any one of clauses 40 to 43, wherein increasing FADS2 activity comprises increasing activity of an endogenous FADS2 gene and/or polypeptide in the subject.
   45. The method according to any one of clauses 40 to 44, wherein increasing FADS2 activity comprises increasing expression of an endogenous FADS2 of the subject.
   46. The method according to any one of clauses 40 to 45, wherein increasing FADS2 activity comprises administering a FADS2 agonist to the subject.
   47. The method according to clause 46, wherein the FADS2 agonist comprises a FADS2 agonist according to any one of clauses 35 to 39.
   48 The FADS2 agonist for use according to any one of clauses 30 to 39, or the methods according to any one of clauses 40 to 47, wherein increasing FADS2 activity and/or the FADS2 agonist increase the amount of triacylglycerides comprising a chain length of at least 54 carbons in the subject.
   49. The FADS2 agonist for use according to any one of clauses 30 to 39 and 48, or the methods according to any one of clauses 40 to 48, wherein increasing FADS2 activity and/or the FADS2 agonist increase the amount of unsaturated triacylglycerides and/or increase the level of unsaturation of triacylglycerides in the subject
   50. The FADS2 agonist for use according to any one of clauses 30 to 39, 48 and 49, or the methods according to any one of clauses 40 to 49, wherein increasing FADS2 activity and/or the FADS2 agonist decrease the amount of fatty acids in the subject.
   51. The FADS2 agonist for use according to any one of clauses 30 to 39, and 48 to 50, or the methods according to any one of clauses 40 to 50, wherein increasing FADS2 activity and/or the FADS2 agonist decrease the de novo lipogenesis (DNL) index of the subject
   52. The FADS2 agonist for use according to any one of clauses 30 to 39, and 48 to 51, or the methods according to any one of clauses 40 to 51, wherein the subject comprises at least one of:
at least one modified PNPLA3 gene;
at least one modified FADS2 gene;
at least one modified APOB gene; and/or
at least one modified MTTP.
   53. The FADS2 agonist for use according to any one of clauses 30 to 39, and 48 to 52, or the methods according to any one of clauses 40 to 52, wherein subject suffers from a monogenic lipid disorder; optionally wherein monogenic lipid disorder is selected from one or more of: familial hypobetalipoproteinaemia (FHBL) and abetalipoproteinemia (ABL).

SEQUENCES

| | SEQ ID |
|---|---|
| Sequence (5′-3′) | NO |
| CTTCCCTAATCTCTTCAGAC | 1 |
| GACAGATTTCTTACACTCCC | 2 |
| AGAACAGGTCTTCGGCTTCG | 3 |
| GAAGAGTACGCACGTACTGT | 4 |
| TGTTACCTCTGCAGGCGCAT | 5 |
| ATCATTCAGCTTCACACAC | 6 |
| CAGCCAGTGCACCCTGAAAG | 7 |
| GAGCCGGTGCGGCAATTTGT | 8 |
| GACCTGCGCTGTCGCGCGAG | 9 |
| CCAGACTTACGTTCTTGCCG | 10 |
| GGACAACCTCATCGGCGGTG | 11 |
| CGCACCATGTGGTTCGCCGG | 12 |
| GAACGGGATGACATTTTACG | 13 |
| GAAAGTGAGCACATATTGGT | 14 |
| TGACCAGTTGATCCAAATAA | 15 |
| GTGCAGAGTGACGCTCAGGG | 16 |
| GGTGCTAGCCTTGCGTTCCG | 17 |
| GCGCATGTTGTTCGGCGCTT | 18 |
| CAACAAAGGCCTCACCTTGC | 19 |
| CCTGCGGCTTCTCGCCCTTC | 20 |
| GAAAACCACGTTGCGGTCCT | 21 |
| AATAGTGGCCGTTGTCTCAG | 22 |
| ATGAACCTCCAGTTTCATCG | 23 |
| GCCCACTTCGAGCTCGTTTC | 24 |
| GAGTTTTATGCTCAGATCGC | 25 |
| GGATAAGGCCACTGTAGAAG | 26 |
| GGATAAGGCCACTGTAGAAG | 27 |
| AAGGATCAGGAAAATTA | 28 |
| GCTTCATGCCCTTCTACAGTGGCCTTA | 29 |
| GCTTCTAGCCCTTCTACAGTGGCCTTA | 30 |

SEQ ID NO: 31-Human FADS2
isoform 1
MGKGGNQGEGAAEREVSVPTFSWEEIQKHN
LRTDRWLVIDRKVYNITKWSIQHPGGQRVI
GHYAGEDATDAFRAFHPDLEFVGKFLKPLL
IGELAPEEPSQDHGKNSKITEDFRALRKTA
EDMNLFKTNHVFFLLLLAHIIALESIAWFT
VFYFGNGWIPTLITAFVLATSQAQAGWLQH
DYGHLSVYRKPKWNHLVHKFVIGHLKGASA
NWWNHRHFQHHAKPNIFHKDPDVNMLHVFV
LGEWQPIEYGKKKLKYLPYNHQHEYFFLIG
PPLLIPMYFQYQIIMTMIVHKNWVDLAWAV
SYYIRFFITYIPFYGILGALLFLNFIRFLE
SHWFVWVTQMNHIVMEIDQEAYRDWFSSQL
TATCNVEQSFFNDWFSGHLNFQIEHHLFPT
MPRHNLHKIAPLVKSLCAKHGIEYQEKPLL
RALLDIIRSLKKSGKLWLDAYLHK
SEQ ID NO: 32-Human FADS2
isoform 2
MHGREAGPFVCVCVLLASIPTPQTPLLQAS
LPPFHPASAGHPITGQQDAFRAFHPDLEFV
GKFLKPLLIGELAPEEPSQDHGKNSKITED
FRALRKTAEDMNLFKTNHVFFLLLLAHIIA
LESIAWFTVFYFGNGWIPTLITAFVLATSQ
AQAGWLQHDYGHLSVYRKPKWNHLVHKFVI
GHLKGASANWWNHRHFQHHAKPNIFHKDPD
VNMLHVFVLGEWQPIEYGKKKLKYLPYNHQ
HEYFFLIGPPLLIPMYFQYQIIMTMIVHKN
WVDLAWAVSYYIRFFITYIPFYGILGALLF
LNFIRFLESHWFVWVTQMNHIVMEIDQEAY
RDWFSSQLTATCNVEQSFFNDWFSGHLNFQ
IEHHLFPTMPRHNLHKIAPLVKSLCAKHGI
EYQEKPLLRALLDIIRSLKKSGKLWLDAYL
HK
SEQ ID NO: 33-Human FADS2
isoform 3
MGKGGNQGEGAAEREVSVPTFSWEEIQKHN
LRTDRWLVIDRKVYNITKWSIQHPGGQRVI
GHYAGEDATDAFRAFHPDLEFVGKFLKPLL
IGELAPEEPSQDHGKNSKITEDFRALRKTA
EDMNLFKTNHVFFLLLLAHIIALESIAWFT
VFYFGNGWIPTLITAFVLATSQAQAGWLQH
DYGHLSVYRKPKWNHLVHKFVIGHLKGASA
NWWNHRHFQHHAKPNIFHKDPDVNMLHVFV
LGEWQPIEYGKKKLKYLPYNHQHEYFFLIG
PPLLIPMYFQYQIIMTMIVHKNWVDLAWAV
SYYIRFFITYIPFYGILGALLFLNFIRFLE
SHWFVWVTQMNHIVMEIDQEAYRDWFSSQL
TATCNVEQSFFNDWFSGHLNFQIEHQ
SEQ ID NO: 34-Human FADS2
isoform 4
MTREPPGCRRVNSLMLYTLRSITSHRSSHP
ERWATSSQDAFRAFHPDLEFVGKFLKPLLI
GELAPEEPSQDHGKNSKITEDFRALRKTAE
DMNLFKTNHVFFLLLLAHIIALESIAWFTV
FYFGNGWIPTLITAFVLATSQAQAGWLQHD
YGHLSVYRKPKWNHLVHKFVIGHLKGASAN
WWNHRHFQHHAKPNIFHKDPDVNMLHVFVL
GEWQPIEYGKKKLKYLPYNHQHEYFFLIGP
PLLIPMYFQYQIIMTMIVHKNWVDLAWAVS
YYIRFFITYIPFYGILGALLFLNFIRFLES
HWFVWVTQMNHIVMEIDQEAYRDWFSSQLT
ATCNVEQSFFNDWFSGHLNFQIEHHLFPTM
PRHNLHKIAPLVKSLCAKHGIEYQEKPLLR
ALLDIIRSLKKSGKLWLDAYLHK
SEQ ID NO: 35-FADS2 fw
5′ GACCACGGCAAGAACTCAAAG 3′
SEQ ID NO: 36-FADS2 rev:
5′ GAGGGTAGGAATCCAGCCATT 3′

1. A human hepatocyte organoid comprising at least one of:
a modified Microsomal Triglyceride Transfer Protein (MTTP) gene;
a modified Apolipoprotein B-100 (APOB) gene;
a modified FADS2 gene; and/or
a modified PNPLA3 gene.

2. The human hepatocyte organoid of claim 1, comprising:
a) a modified Apolipoprotein B-100 (APOB) gene;
b) a modified Microsomal Triglyceride Transfer Protein (MTTP) gene;
c) a modified FADS2 gene;
d) a modified PNPLA3 gene;
e) a modified Apolipoprotein B-100 (APOB) gene and a modified FADS2 gene;
f) a modified Apolipoprotein B-100 (APOB) gene and a modified PNPLA3 gene;
g) a modified Microsomal Triglyceride Transfer Protein (MTTP) gene and a modified FADS2 gene; or
h) a modified Microsomal Triglyceride Transfer Protein (MTTP) gene and a modified PNPLA3 gene.

3. The human hepatocyte organoid of claim 1 or 2, wherein the modification comprises a mutation or deletion; optionally wherein at least one of the modified Apolipoprotein B-100 (APOB) gene, the modified Microsomal Triglyceride Transfer Protein (MTTP) gene, the modified FADS2 gene, and/or the modified PNPLA3 gene are attenuated.

4. The human hepatocyte organoid of any of claims 1 to 3, wherein the human hepatocyte organoid comprise lipids droplets, wherein the lipid droplets occupy a greater area of the human hepatocyte organoid in comparison to a wild type human hepatocyte organoid.

5. The human hepatocyte organoid of any preceding claim, wherein human hepatocyte organoid comprises altered lipid homeostasis.

6. The human hepatocyte organoid of any preceding claim, wherein the human hepatocyte organoid accumulates lipids via de novo lipogenesis-driven steatosis; optionally wherein the human hepatocyte organoid is according to any of claim 2 a), b), e), f), g), or h).

7. The human hepatocyte organoid of any preceding claim, wherein the human hepatocyte organoid is a tissue derived human hepatocyte organoid.

8. The human hepatocyte organoid of any preceding claim, wherein the human hepatocyte organoid further comprises exogenous lipids.

9. The human hepatocyte organoid of claim 2 a), b), e), f), g), or h) comprising downregulation of at least one LXR-regulated gene in comparison to a wild type human hepatocyte organoid.

10. The human hepatocyte organoid of claim 9, wherein the at least one LXR-regulated gene comprises one or more of ACACA, FASN, DGAT2, SREBF1, HMGCS1, SQLE, LSS, and/or DHCR7.

11. A method of forming a human hepatocyte organoid for modelling lipid homeostasis, the method comprising:
a. providing a human hepatocyte organoid;
b. modifying at least one of:
i. at least one Microsomal Triglyceride Transfer Protein (MTTP) gene;
ii. at least one Apolipoprotein B-100 (APOB) gene;
iii. at least one FADS2 gene; and/or
iv. at least one PNPLA3 gene;
c. recovering cells comprising the modified APOB, MTTP, FADS2, and/or PNPLA3 genes; and
d. culturing the cells to form human hepatocyte organoids.

12. The method of claim 11, wherein modifying comprises CRISPR based gene disruption.

13. The method of claim 12, wherein CRISPR based gene disruption comprises introducing into cells of the human hepatocyte organoid one or more vectors for disrupting the APOB, MTTP FADS2, and/or PNPLA3 genes, the at least one vector comprising at least one of a guide RNA for targeting APOB, MTTP FADS2, and/or PNPLA3 and/or a Cas9 enzyme.

14. Use of a human hepatocyte organoid according to any of claims 1 to 10 or formed by the methods of any of claims 11 to 13 for modelling lipid homeostasis; optionally wherein the use further comprises drug discovery and/or CRISPR based screening of lipid homeostasis mediators.

15. The use of claim 14, wherein the human hepatocyte organoid is for modelling steatosis.

16. The use of claim 15, wherein the steatosis is de novo lipogenesis driven steatosis.

17. The use of claims 14 to 16, wherein the human hepatocyte organoid is for modelling NAFLD, NASH and/or liver cancer.

18. A p38 inhibitor, FADS2 agonist, ACC inhibitor, DGAT2 inhibitor, FAS inhibitor recombinant hFGF19 or FXR agonist for use in treating NAFLD in a subject in need thereof, wherein the subject comprises at least one of:
a. at least one modified MTTP gene;
b. at least one modified FADS2 gene;
c. at least one modified APOB gene; and/or
d. at least one modified PNPLA3 gene.

19. The p38 inhibitor, FADS2 agonist, ACC inhibitor, FXR agonist, DGAT2 inhibitor, FAS inhibitor or hFGF19 for use according to claim 18, wherein the subject suffers from familial hypobetalipoproteinaemia (FHBL).

20. The p38 inhibitor, FADS2 agonist, ACC inhibitor, FXR agonist, DGAT2 inhibitor, FAS inhibitor or hFGF19 for use according to claim 19, wherein the familial hypobetalipoproteinaemia is associated with the least one attenuating APOB mutation.

21. The p38 inhibitor, FADS2 agonist, ACC inhibitor, FXR agonist, DGAT2 inhibitor, FAS inhibitor or hFGF19 for use according to any one of claims 18 to 20, wherein the subject suffers from abetalipoproteinemia (ABL).

22. The p38 inhibitor, FADS2 agonist, ACC inhibitor, FXR agonist, DGAT2 inhibitor, FAS inhibitor or hFGF19 for use according to claim 21, wherein the abetalipoproteinemia is associated with the at least one attenuating MTTP mutation.

23. The p38 inhibitor, FADS2 agonist, ACC inhibitor, FXR agonist, DGAT2 inhibitor, FAS inhibitor or hFGF19 for use according to any one of claims 18 to 22 wherein the at least one modified PNPLA3 comprises a homozygous or heterozygous PNPLA3 I148M mutation.

24. The p38 inhibitor, FADS2 agonist, ACC inhibitor, FXR agonist, DGAT2 inhibitor, FAS inhibitor or hFGF19 for use according to any one of claims 18 to 23, wherein the at least one modified FADS2 comprises a single nucleotide polymorphism.

25. A method reducing steatosis in hepatocytes in a subject in need thereof, comprising administering an agent targeting de novo lipogenesis to the subject, wherein the subject comprises at least one of:
a. at least one modified MTTP gene;
b. at least one modified FADS2 gene;
c. at least one modified APOB gene; and/or
d. at least one modified PNPLA3 gene.

26. The method according to claim 25, wherein the agent comprises at least one of:
p38 inhibitor;
FADS2 agonist;
ACC inhibitor;
FXR agonist;
FAS inhibitor;
DGAT2 inhibitor; and/or
recombinant hFGF19.

27. The method according to any of claim 25 or 26, wherein the subject suffers from NAFLD.

28. A method of treating NAFLD comprising inducing Dual Specificity Phosphatase 4 and/or Dual Specificity Phosphatase 5 in a subject in need thereof.

29. The method of claim 28, wherein inducing Dual Specificity Phosphatase 4 and/or Dual Specificity Phosphatase 5 comprises administering an agent that inhibits p38 signalling.

30. A method of treating or preventing a cardiovascular disease in a subject in need thereof, comprising increasing FADS2 activity in the subject.

31. A method of reducing and/or preventing steatosis in a subject in need thereof, comprising increasing FADS2 activity in the subject.

32. A method of preventing and/or reducing the risk of NASH and/or cirrhosis in a subject in need thereof, comprising increasing FADS2 activity in the subject.

33. A FADS2 agonist for use in treating or preventing a cardiovascular disease in a subject in need thereof.

34. The method according to any one of claims 30 to 33 wherein increasing FADS2 activity comprises:
a. increasing activity of an endogenous FADS2 gene and/or polypeptide in the subject;
b. increasing expression of an endogenous FADS2 of the subject; and/or
c. administering a FADS2 agonist to the subject.

35. A FADS2 agonist for use in reducing and/or preventing steatosis in a subject in need thereof; optionally wherein the steatosis is dietary induced steatosis.

36. A FADS2 agonist for use in preventing and/or reducing the risk of NASH and/or cirrhosis in a subject in need thereof.

37. The FADS2 agonist for use according to any of claims 18 to 24, 35 and 36 or the method according to any one of claims 25 to 32, wherein the FADS2 agonist comprises an agent for increasing activity of FADS2 in the subject; optionally wherein the FADS2 agonist comprises an agent for increasing expression of endogenous and/or exogenous FADS2 in the subject.

38. The FADS2 agonist for use according to any of claim 37 or the method according to any one of claim 37, wherein the agent comprises:
a. a nucleic acid that encodes a FADS2 agonist; optionally wherein the FADS2 agonist comprises a nucleic acid that encodes an endogenous and/or exogenous FADS2 polypeptide; optionally wherein the FADS2 polypeptide comprises a human FADS2 polypeptide;
b. a polypeptide encoding an exogenous and/or endogenous FADS2 polypeptide optionally wherein the FADS2 polypeptide comprises a human FADS2 polypeptide; or
c. an expression vector, the expression vector comprising a nucleic acid or FADS2 polypeptide according to a. or b.

39. The FADS2 agonist for use according to any of claims 18 to 24, 35 to 38 or the method according to any one of claims 25 to 32 and 37 to 38, wherein increasing FADS2 activity and/or the FADS2 agonist increase:
a. the amount of triacylglycerides comprising a chain length of at least 54 carbons in the subject;
b. the amount of unsaturated triacylglycerides and/or increase the level of unsaturation of triacylglycerides in the subject;
c. decrease the amount of fatty acids in the subject;
d. decrease the de novo lipogenesis (DNL) index of the subject.

40. The FADS2 agonist for use according to any of claims 18 to 24, 35 to 39 or the method according to any one of claims 25 to 32 and 37 to 39, wherein the subject comprises at least one of:
a. at least one modified MTTP
b. at least one modified PNPLA3 gene;
c. at least one modified FADS2 gene; and/or
d. at least one modified APOB gene.


SEQUENCE LISTING (36 sequences)

SEQ ID NO: 1 - DNA, 20 bp, synthetic construct
  misc_feature 1..20: note = ACACA-KO-sgRNA spacer
  source 1..20: mol_type = other DNA
cttccctaatctcttcagac

SEQ ID NO: 2 - DNA, 20 bp, synthetic construct
  misc_feature 1..20: note = ACACB-KO-sgRNA spacer
  source 1..20: mol_type = other DNA
gacagatttcttacactccc

SEQ ID NO: 3 - DNA, 20 bp, synthetic construct
  misc_feature 1..20: note = ACE2-KO-sgRNA
  source 1..20: mol_type = other DNA
agaacaggtcttcggcttcg

SEQ ID NO: 4 - DNA, 20 bp, synthetic construct
  misc_feature 1..20: note = ACSL1-KO-sgRNA spacer
  source 1..20: mol_type = other DNA
gaagagtacgcacgtactgt

SEQ ID NO: 5 - DNA, 20 bp, synthetic construct
  misc_feature 1..20: note = AKR1C1-KO-sgRNA spacer
  source 1..20: mol_type = other DNA
tgttacctctgcaggcgcat

SEQ ID NO: 6 - DNA, 19 bp, synthetic construct
  misc_feature 1..19: note = AKR1C2-KO-sgRNA spacer
  source 1..19: mol_type = other DNA
atcattcagcttcacacac

SEQ ID NO: 7 - DNA, 20 bp, synthetic construct
  misc_feature 1..20: note = APOB-KO-sgRNA spacer
  source 1..20: mol_type = other DNA
cagccagtgcaccctgaaag

SEQ ID NO: 8 - DNA, 20 bp, synthetic construct
  misc_feature 1..20: note = BHLHE40-KO-sgRNA spacer
  source 1..20: mol_type = other DNA
gagccggtgcggcaatttgt

SEQ ID NO: 9 - DNA, 20 bp, synthetic construct
  misc_feature 1..20: note = DGAT2-KO-sgRNA spacer
  source 1..20: mol_type = other DNA
gacctgcgctgtcgcgcgag

SEQ ID NO: 10 - DNA, 20 bp, synthetic construct
  misc_feature 1..20: note = FADS2-KO-sgRNA spacer
  source 1..20: mol_type = other DNA
ccagacttacgttcttgccg

SEQ ID NO: 11 - DNA, 20 bp, synthetic construct
  misc_feature 1..20: note = FASN-KO-sgRNA spacer
  source 1..20: mol_type = other DNA
ggacaacctcatcggcggtg

SEQ ID NO: 12 - DNA, 20 bp, synthetic construct
  misc_feature 1..20: note = LPCAT1-KO-sgRNA spacer
  source 1..20: mol_type = other DNA
cgcaccatgtggttcgccgg

SEQ ID NO: 13 - DNA, 20 bp, synthetic construct
  misc_feature 1..20: note = LSS-KO-sgRNA spacer
  source 1..20: mol_type = other DNA
gaacgggatgacattttacg

SEQ ID NO: 14 - DNA, 20 bp, synthetic construct
  misc_feature 1..20: note = MOGAT3-KO-sgRNA, spacer
  source 1..20: mol_type = other DNA
gaaagtgagcacatattggt

SEQ ID NO: 15 - DNA, 20 bp, synthetic construct
  misc_feature 1..20: note = MTTP-KO-sgRNA spacer
  source 1..20: mol_type = other DNA
tgaccagttgatccaaataa

SEQ ID NO: 16 - DNA, 20 bp, synthetic construct
  misc_feature 1..20: note = MVD-KO-sgRNA spacer
  source 1..20: mol_type = other DNA
gtgcagagtgacgctcaggg

SEQ ID NO: 17 - DNA, 20 bp, synthetic construct
  misc_feature 1..20: note = PCKS9-KO-sgRNA spacer
  source 1..20: mol_type = other DNA
ggtgctagccttgcgttccg

SEQ ID NO: 18 - DNA, 20 bp, synthetic construct
  misc_feature 1..20: note = PNPLA3-KO-sgRNA, spacer
  source 1..20: mol_type = other DNA
gcgcatgttgttcggcgctt

SEQ ID NO: 19 - DNA, 20 bp, synthetic construct
  misc_feature 1..20: note = PLIN1-KO-sgRNA spacer
  source 1..20: mol_type = other DNA
caacaaaggcctcaccttgc

SEQ ID NO: 20 - DNA, 20 bp, synthetic construct
  misc_feature 1..20: note = SCARB1-KO-sgRNA, spacer
  source 1..20: mol_type = other DNA
cctgcggcttctcgcccttc

SEQ ID NO: 21 - DNA, 20 bp, synthetic construct
  misc_feature 1..20: note = SERPINE1-KO-sgRNA, spacer
  source 1..20: mol_type = other DNA
gaaaaccacgttgcggtcct

SEQ ID NO: 22 - DNA, 20 bp, synthetic construct
  misc_feature 1..20: note = SLC29A3-KO-sgRNA, spacer
  source 1..20: mol_type = other DNA
aatagtggccgttgtctcag

SEQ ID NO: 23 - DNA, 20 bp, synthetic construct
  misc_feature 1..20: note = STARD8-KO-sgRNA spacer
  source 1..20: mol_type = other DNA
atgaacctccagtttcatcg

SEQ ID NO: 24 - DNA, 20 bp, synthetic construct
  misc_feature 1..20: note = TRIB3-KO-sgRNA spacer
  source 1..20: mol_type = other DNA
gcccacttcgagctcgtttc

SEQ ID NO: 25 - DNA, 20 bp, synthetic construct
  misc_feature 1..20: note = PLIN2-KI-sgRNA spacer
  source 1..20: mol_type = other DNA
gagttttatgctcagatcgc

SEQ ID NO: 26 - DNA, 20 bp, synthetic construct
  misc_feature 1..20: note = PNPLA3-I148M-pegRNA spacer
  source 1..20: mol_type = other DNA
ggataaggccactgtagaag

SEQ ID NO: 27 - DNA, 20 bp, synthetic construct
  misc_feature 1..20: note = PNPLA3-I148*-pegRNA spacer
  source 1..20: mol_type = other DNA
ggataaggccactgtagaag

SEQ ID NO: 28 - DNA, 20 bp, synthetic construct
  misc_feature 1..20: note = PNPLA3-I148-PE3-sgRNA spacer
  source 1..20: mol_type = other DNA
aaggatcaggaaaattaaaa

SEQ ID NO: 29 - DNA, 27 bp, synthetic construct
  misc_feature 1..27: note = PNPLA3-I148M-pegRNA 3- extension
  source 1..27: mol_type = other DNA
gcttcatgcccttctacagtggcctta

SEQ ID NO: 30 - DNA, 27 bp, synthetic construct
  misc_feature 1..27: note = PNPLA3-I148*-pegRNA 3- extension
  source 1..27: mol_type = other DNA
gcttctagcccttctacagtggcctta

SEQ ID NO: 31 - protein, 444 aa, Homo sapiens
  REGION 1..444: note = Human FADS2 isoform 1
MGKGGNQGEGAAEREVSVPTFSWEEIQKHNLRTDRWLVIDRKVYNITKWSIQHPGGQRVIGHYAGEDATDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHHLFPTMPRHNLHKIAPLVKSLCAKHGIEYQEKPLLRALLDIIRSLKKSGKLWLDAYLHK

SEQ ID NO: 32 - protein, 422 aa, Homo sapiens
  REGION 1..422: note = Human FADS2 isoform 2
MHGREAGPFVCVCVLLASIPTPQTPLLQASLPPFHPASAGHPITGQQDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHHLFPTMPRHNLHKIAPLVKSLCAKHGIEYQEKPLLRALLDIIRSLKKSGKLWLDAYLHK

SEQ ID NO: 33 - protein, 386 aa, Homo sapiens
  REGION 1..386: note = Human FADS2 isoform 3
MGKGGNQGEGAAEREVSVPTFSWEEIQKHNLRTDRWLVIDRKVYNITKWSIQHPGGQRVIGHYAGEDATDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHQ

SEQ ID NO: 34 - protein, 413 aa, Homo sapiens
  REGION 1..413: note = Human FADS2 isoform 4
MTREPPGCRRVNSLMLYTLRSITSHRSSHPERWATSSQDAFRAFHPDLEFVGKFLKPLLIGELAPEEPSQDHGKNSKITEDFRALRKTAEDMNLFKTNHVFFLLLLAHIIALESIAWFTVFYFGNGWIPTLITAFVLATSQAQAGWLQHDYGHLSVYRKPKWNHLVHKFVIGHLKGASANWWNHRHFQHHAKPNIFHKDPDVNMLHVFVLGEWQPIEYGKKKLKYLPYNHQHEYFFLIGPPLLIPMYFQYQIIMTMIVHKNWVDLAWAVSYYIRFFITYIPFYGILGALLFLNFIRFLESHWFVWVTQMNHIVMEIDQEAYRDWFSSQLTATCNVEQSFFNDWFSGHLNFQIEHHLFPTMPRHNLHKIAPLVKSLCAKHGIEYQEKPLLRALLDIIRSLKKSGKLWLDAYLHK

SEQ ID NO: 35 - DNA, 21 bp, synthetic construct
  misc_feature 1..21: note = FADS2_fw
  source 1..21: mol_type = other DNA
gaccacggcaagaactcaaag

SEQ ID NO: 36 - DNA, 21 bp, synthetic construct
  misc_feature 1..21: note = FADS2_rev
  source 1..21: mol_type = other DNA
gagggtaggaatccagccatt